(19) United States
(12) Patent Application Publication
(10) Pub. No.: US 2026/0041718 A1
(43) Pub. Date: Feb. 12, 2026

(54) COMPOSITIONS, DEVICES, AND METHODS RELATING TO USE OF AN AMNIOTIC MEMBRANE FETAL TISSUE PRODUCT

(71) Applicant: BioTissue Holdings Inc., Miami, FL (US)

(72) Inventors: Ek Kia TAN, Pinecrest, FL (US); Sean TIGHE, Fort Lauderdale, FL (US); Scheffer TSENG, Pinecrest, FL (US)

(21) Appl. No.: 19/353,469

(22) Filed: Oct. 8, 2025

Related U.S. Application Data
(63) Continuation of application No. PCT/US2025/031892, filed on Jun. 2, 2025.
(60) Provisional application No. 63/655,526, filed on Jun. 3, 2024.

(51) Int. Cl. A61K 35/545; A61K 9/00; A61K 47/34; A61P 27/02

(52) U.S. Cl. CPC A61K 35/545; A61K 9/0051; A61K 47/34; A61P 27/02

(57) ABSTRACT

This present disclosure describes an amniotic membrane fetal tissue product with adhesive properties for use on an ocular surface to prevent or treat eye indications.

### CROSS REFERENCE

This application is a continuation of International Application No. PCT/US2025/031892, filed Jun. 2, 2025, which claims the benefit of U.S. Provisional Application No. 63/655,526 filed Jun. 3, 2024, all of which are hereby incorporated by reference in their entireties.

### SUMMARY

Disclosed herein are fetal tissue products comprising placental amniotic membrane (PAM) compositions, and methods, kits, and devices comprising PAM. Fetal tissue products comprising PAM provided herein have increased adhesiveness to a surface e.g., surface of an eye or an artificial contact lens or collagen shield and retain HC-HA/PTX3 complex and its biological activities. Fetal tissue products comprising PAM provided herein have significant reduction of total hyaluronic acid (HA) content and reduction of the molecular weight of HA. In some embodiments, the increased adhesiveness of the fetal tissue product comprising PAM arises from the significant reduction of total HA, which facilitates lubrication. The compositions may advantageously be used to treat eye injuries, disorders or diseases with fewer side effects such as ulcerations or cellular or inflammatory infiltrates to sensitive areas of the eye, such as the cornea. Additionally, the products described herein are shelf-stable and can be shipped and stored at room temperature allowing for more convenient distribution and storage.

Disclosed herein are amniotic membrane compositions with advantageous properties, such as high levels of adhesiveness that allow for ease of direct administration to an eye of a patient or individual or adheres when applied to a surface that can then be administered to an individual. Such surfaces may comprise a bandage, collagen shield, a dressing, a wound covering, a hydrogel, or a contact lens. Such adhesive properties, which may be related to low hyaluronic acid (HA) content of the composition, are especially well adapted to treating ophthalmological indications which require high adhesiveness to maintain contact to the eye. Importantly, an amniotic membrane composition still comprises HC-HA/PTX3 which is responsible for the therapeutic benefit of these compositions.

Disclosed herein in some embodiments are compositions, methods, devices comprising a fetal tissue product comprising fetal tissue. In some embodiments, a fetal tissue product comprises an amniotic membrane (AM) product. In some embodiments, a fetal tissue product can comprise less than about 30 micrograms of hyaluronic acid (HA) per 32 cm². In some embodiments, a fetal tissue product can be a sheet. In some embodiments, a fetal tissue product can comprise less than about 20 micrograms of hyaluronic acid per 32 cm². In some embodiments, a fetal tissue product can comprise less than about 20 micrograms of hyaluronic acid per 32 cm². In some embodiments, the HA can be a low molecular weight HA. In some embodiments, a fetal tissue product can comprise about 20% to about 90% of the HA is covalently bonded to a heavy chain 1 of IαI (HC1) complexed with pentraxin 3 (PTX3). In some embodiments, a fetal tissue product can comprise about 30% to about 75% of the HA is covalently bonded to a heavy chain 1 of IαI (HC1) complexed with pentraxin 3 (PTX3). In some embodiments, a fetal tissue product can comprise about 20% to about 50% of the HA is covalently bonded to a heavy chain 1 of IαI (HC1) complexed with pentraxin 3 (PTX3). In some embodiments, a fetal tissue product can comprise about 20% to about 40% of the HA is covalently bonded to a heavy chain 1 of IαI (HC1) complexed with pentraxin 3 (PTX3). In some embodiments, a fetal tissue product can comprise amniotic membrane (AM). In some embodiments, a fetal tissue product can comprise placental amniotic membrane (AM). In some embodiments, a fetal tissue product can comprise a fetal tissue that was previously frozen. In some embodiments, a fetal tissue product can comprise a sheet that is circular. In some embodiments, a fetal tissue product can comprise a sheet having a diameter from 8 millimeters to 16 millimeters. In some embodiments, a fetal tissue product can comprise a sheet having a diameter from 9 millimeters to 15 millimeters. In some embodiments, a fetal tissue product can comprise a sheet having a diameter from 10 millimeters to 14 millimeters. In some embodiments, a fetal tissue product can comprise a sheet having a diameter from 11 millimeters to 13 millimeters. In some embodiments, a fetal tissue product can comprise a sheet having a diameter of about 12 millimeters. In some embodiments, a fetal tissue product can comprise a sheet having an average thickness of less than about 200 micrometers. In some embodiments, a fetal tissue product can comprise a sheet having an average thickness of between about 150 micrometers and about 75 micrometers. In some embodiments, a fetal tissue product can comprise a sheet having an average thickness of greater than about 50 micrometers. In some embodiments, a fetal tissue product can comprise a sheet that is substantially flat. In some embodiments, a fetal tissue product can comprise a fetal tissue that is not dehydrated. In some embodiments, a fetal tissue product can comprise water that is not removed from the fetal tissue. In some embodiments, a fetal tissue product can comprise a fetal tissue that is terminally sterilized. In some embodiments, a fetal tissue product can comprise a fetal tissue that is terminally sterilized by gamma irradiation or electron beam sterilization. In some embodiments, a fetal tissue product can comprise substantially all of the cells of a fetal tissue that are dead. In some embodiments, a fetal tissue product can be terminally sterilized at a temperature at or below 0° C. In some embodiments, a fetal tissue product can adhere to a backing material. In some embodiments, a fetal tissue product can comprise a backing material that comprises a polyethersulfone (PES) polymer. In some embodiments, a fetal tissue product can comprise a backing material that is gridded. In some embodiments, a fetal tissue product may not comprise a ring-shaped support structure. In some embodiments, a fetal tissue product can be clear. In some embodiments, a fetal tissue product can be optically clear. In some embodiments, a fetal tissue product can be packaged in normal (0.9%) saline. In some embodiments, a fetal tissue product can be stable at −20° C. to 25° C. for at least one year. In some embodiments, a fetal tissue product can be stable at 20° C. to 25° C. for at least one year. In some embodiments, a fetal tissue product can adhere to a surface comprising lotrafilcon A with a force of greater than 0.0001 pounds per square inch. In some embodiments, a fetal tissue product can adhere to a surface comprising lotrafilcon A with a force of greater than 0.001 pounds per square inch. In some embodiments, a fetal tissue product can adhere to a surface comprised of lotrafilcon A with a force of greater than 0.01 pounds per square inch. In some embodiments, a fetal tissue product can adhere to a surface comprising lotrafilcon A with a force of greater than 0.1 pounds per square inch. In some embodiments, a fetal tissue product can adhere to a surface comprising lotrafilcon A with a force of greater than 1 pound per square inch. In some embodiments, a fetal tissue product can adhere to a surface comprising lotrafilcon A with a force of greater than 2 pounds per square inch. In some embodiments, a fetal tissue product can adhere to a surface comprising lotrafilcon A with a force of greater than 5 pounds per square inch. In some embodiments, a fetal tissue product can adhere to a surface comprising lotrafilcon A with a force of greater than 10 pounds per square inch. In some embodiments, a fetal tissue product can adhere to a surface comprising lotrafilcon A with a force of greater than 15 pounds per square inch. In some embodiments, a fetal tissue product can adhere to a surface comprised of lotrafilcon A with a force of greater than 20 pounds per square inch. In some embodiments, a fetal tissue product can exhibit increased adhesion to a surface comprising lotrafilcon A after sterilization with gamma irradiation at temperature of below Oo C. In some embodiments, a fetal tissue product can adhere to the contact lens such that when inserted into an eye of an individual the fetal tissue product contacts a surface of an eye of an individual. In some embodiments disclosed herein is a method of treating a disease or disorder of an eye comprising administering a fetal tissue product that can adhere to a surface of a contact lens to an eye of an individual. In some embodiments, a method of administering a fetal tissue product to an eye can be done without adhering the fetal tissue product to a surface of a contact lens. In some embodiments, a method of administering a fetal tissue product to the eye can comprise adhering the fetal tissue product to the surface of a contact lens. In some embodiments, a method of treating a disease or a disorder of an eye where the disease or the disorder of the eye can comprise a dry eye, a corneal abrasion, a neurotrophic keratitis, a corneal epithelial defect, a corneal ulcer, or a recurrent corneal epithelial erosion. In some embodiments, a method of treating a disease or a disorder of an eye can comprise dry eye disease. In some embodiments, a method of treating a disease or a disorder of an eye can comprise a corneal abrasion. In some embodiments, a method of making a treatment for a disease of an eye can comprise contacting a fetal tissue product to a surface of a contact lens, thereby adhering the fetal tissue product to the contact lens. In some embodiments, disclosed herein is a kit for treating a disease or disorder of an eye. In some embodiments, a kit can comprise a fetal tissue product and any one or more of tweezers, a swab, and a surgical tray. In some embodiments, a kit can comprise a fetal tissue that is adhered to a backing material. In some embodiments, a backing material can be a polyethersulfone (PES) polymer. In some embodiments, a backing material can be gridded. In some embodiments, a kit can comprise a fetal tissue for treating a disease or a disorder of an eye. In some embodiments, a disease or a disorder of an eye can comprise dry eye disease. In some embodiments, a disease or a disorder of an eye can comprise a corneal abrasion. In some embodiments, a disease or disorder of an eye can comprise a dry eye, a corneal abrasion, a neurotrophic keratitis, a corneal epithelial defect, a corneal ulcer, or a recurrent corneal epithelial erosion.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates biological mechanisms of the fetal tissue product/AM product.

FIG. 2A shows the results of a hyaluronic acid (HA) quantitative assay performed to determine HA content of amniotic membrane extract (AME) derived from four different amniotic membrane sheet products. The quantitative HA assay conducted revealed the AM product of the present disclosure had the lowest amount of HA.

FIG. 2B illustrates an agarose gel electrophoresis performed using AME of four different products to analyze the HA size in four different products. AM product of the present disclosure had the smallest molecular weight.

FIG. 3A-FIG. 3B illustrates a Western blot analysis performed to assess heavy chain 1 (HC1) content in amniotic membrane extract (AME) derived from four different products. The covalent bonded HC1 was released only after digestion of hyaluronidase (Hase). The AM product of the present disclosure retained HC-HA complex that was released by Hase.Densitometry (FIG. 3A) assay of the Western blot showed the percent relative quantities of HC1 (FIG. 3B) in AME of four different products. The AM product of the present disclosure had a higher amount of HC-HA complex than two other products (XcellerEYES™ and Ambio2®, which are both dry and terminally sterilized) although lower than the wet but non-terminally sterilized native AM (i.e., AmnioGraft).

FIG. 4A-FIG. 4D Illustrates a Western blot analysis performed to assess pentraxin 3 (PTX3) content complexed in HC-HA in AME derived from four different types of amniotic membrane (AM) sheet products. Western blot analysis was conducted to assess and confirm preservation of HC-HA/PTX3 different sheet products. The complexed PTX3 was released after digestion of Hase, which released HC-HA as described in FIG. 3A-FIG. 3B. The AM product of the present disclosure retained complexed PTX3 that was released by Hase. Densitometry assay of the Western blot of PTX3 with hyaluronidase digestion (FIG. 4A) and PTX3 with additional DTT reduction (FIG. 4C) showed the percent relative quantities of complexed PTX3 (FIG. 4B) in AME of four different AME sheet products (AmnioGraft™, XcellerEYES™ and Ambio2®, AmbioDisk®), and the AM product or fetal tissue product. The AM product of the present disclosure had a higher amount of complexed PTX3 than two other products (XcellerEYES™ and Ambio2®, which are both dry and terminally sterilized) although lower than the wet but non-terminally sterilized native AM (i.e., AmnioGraft). The potency of HC-HA/PTX3 was assessed by the addition of 25 μg/mL of amniotic membrane extracts (AME) from each tissue product to a nitric oxide (NO) assay and determining the inhibitory activity of the AME of each tissue product at an OD 540 nm (FIG. 4D).

FIG. 5A-FIG. 5F illustrates directions required to aseptically unpack and press the fetal tissue product or the AM product of the present disclosure onto a bandage contact lens (BCL). Directions to open and press the AM product onto an exemplary BCL are demonstrated in steps: 1) open the AM product package to retrieve the product (FIG. 5A); 2) place AM product onto the surgical tray surface (FIG. 5B); 3) use of a forceps to remove the outer packaging, access the AM product in the inner pouch and press onto the lens (FIG. 5C); 4) steps to retrieve and access the contact lens (BCL; FIG. 5D), 5) step to adhere inverted BCL onto the AM product (FIG. 5E), and 6) step to revert the BCL or a contact lens to its normal shape once the AM product has been spread onto the lens (FIG. 5F).

FIG. 6A-FIG. 6B illustrates a close-up view of the AM product as shipped and in unpackaged form which can be seen in FIG. 6A-FIG. 6B. A disc with the AM product on a backing material is shown before detachment (FIG. 6A) and after detachment of the backing material and placement of the AM product on a contact lens (FIG. 6B). As seen in FIGS. 5F and 6B, the lens (1) is held to the AM product (2) by the adhesiveness of the AM product. The AM product may then be administered to the corneal surface using the same technique for inserting a contact lens using an index finger.

FIG. 7A-FIG. 7D illustrates a representative case study demonstrating the use of the AM product of the present disclosure to treat an exemplary eye indication. A female subject presented with moderate superficial punctate keratitis (SPK) and exposure keratitis due to Lagophthalmos, FIG. 7A The subject had not worn contact lenses for >40 years due to persistent ocular dryness and discomfort. To treat the SPK and exposure keratitis due to Lagophthalmos, the disclosed AM product was placed on an adjunctive bandage contact lens (BCL). The BCL was placed on the surface of the eye of the subject for up to 3 days, FIG. 7B. After 3 days, it was determined that (1) there was no AM product in the BCL after removal of the lens confirming the AM product had dissolved into the eye. This case study also demonstrated that (2) the AM product was efficacious and effective in treating the eye indication as it was confirmed for the 3 days that the subject had improved visual acuity, decreased symptoms of ocular dryness, and decreased SPK and/or staining in the area of exposure FIG. 7C-7D.

FIG. 8 illustrates the overall temporal changes and shrinkage of the AM product which capture the tissue dissolution process when the tissue product is adhered to the corneal surface of an eye.

FIG. 9A-FIG. 9B illustrates a close-up view of the AM product adhered on exemplary soft contact lenses. The AM product (fetal tissue product) was placed on a bandage contact lens (FIG. 9A) or a collagen shield (FIG. 9B).

FIG. 10A-FIG. 10H illustrates steps on how to aseptically unpack and place the fetal tissue product or the AM product of the present disclosure onto a Collagen Shield (CS), or Collagen corneal shield contact lens. Directions to open and press the AM product onto an exemplary CS contact lens are demonstrated in steps: 1) Open Collagen Shield (CS) package in a sterile container (FIG. 10A), 2) fully submerge CS in balanced salt solution (BSS) for 30 seconds (FIG. 10B), 3) after CS is hydrated, revert the CS so that it is no longer inside out (FIG. 10C), 4) remove excess fluid from CS and the tray (FIG. 10D), 5) retrieve amnion graft membrane and place on CD, gridded side down (FIG. 10E), 6) use swab and forceps to gently peep off the paper backing (FIG. 10F), 7) use swab to smooth out the amnion graft membrane in the CS (FIG. 10G), 8) amnion graft membrane on the CS, ready to be place on an eye, a forceps is used for demonstration only; a membrane placed on the CS (FIG. 10H).

FIG. 11A-FIG. 11C illustrates stability testing of fetal tissue samples after storage at accelerated conditions. Samples were placed between a grid on each side and were hydrated with storage solution after three months (FIG. 11A), six months (FIG. 11B) and 9 months (FIG. 11C) of storage at accelerated storage conditions of 40° C.±2° C. with 75% relative humidity (RH)+5% RH.

FIG. 12A-FIG. 12C illustrates a survey that assessed the outcomes associated with using the fetal tissue product by age of the study subjects. Cryopreserved amniotic membrane fetal tissue product were placed on subjects in an outpatient study. After each use, a subject completed an experience survey related to product handling and application, patients comfort during treatment, and short-term outcomes (FIG. 12A) related to seeking treatment for various common ocular conditions or diseases (FIG. 12B). The wear time for the tissue product varied from one to seven days with an average wear time of 2.5 days (±1.5) throughout the study (FIG. 12C).

FIG. 13A-FIG. 13C illustrates the overall observed improvement in subjects who participated in using the fetal tissue product to treat the most common ocular indications. Over 94% (258 out of 274) of subject had improvement (FIG. 13A) with 77% (211 out of 274), 68% and 35% showing improved corneal healing, improved symptoms and improved vision, respectively (FIG. 13B). Overall, 98% of subjects that used a collagen shield (CS) or collagen corneal shield and 84% that used a bandage contact lens (BCL) showed improvement (FIG. 13C).

FIG. 14A-FIG. 14F illustrates a study to characterization an internal reference material (IRM) and assess the potency of IRM. Agarose gel electrophoresis (FIG. 14A), Western blot analysis of heavy chain 1 (HC1) (FIG. 14B) and PTX3 (FIG. 14C), phase contrast micrographs and immunostaining of human corneal fibroblast (HCF, FIG. 14D), and assays of polarization of M2 macrophages via measurements of IL-12 (p40) (FIG. 14E) and NO assay (FIG. 14F) were performed on IRM. Passage 3 HCF cells cultured on plastic in DMEM+10% FBS were seeded on plastic for 24 hours before being switched to DMEM/ITS for 24 hours with or without 10 ng/mL TGF-b1 for another 24 hours (FIG. 14D). The cell morphology was analyzed by phase-contrast microscopy at 72 hours after TGF-b1 treatment (scale bar: 50 mm). Immunostaining of pSmad2/3 and alpha-SMA was performed at 24 and 72 hours, respectively (nuclear counterstained by Hoechst 33342, scale bar: 20 mm). Various concentrations of IRM from 1.6 mg/mL of HA were loaded. HCF, human corneal fibroblasts (FIG. 14E, FIG. 14F). DMEM, Dulbecco's Modified Eagle medium; FBS, fetal bovine serum; ITS, insulin-transferrin-selenium.

FIG. 15A-FIG. 15E illustrates selective capture of HC-HC-PTX3 in amniotic membrane extract (AME). Nylon filter was used to capture HC-HA/PTX3 and assess its inhibitory activity in a NO assay conducted on the released (recovered) HC-HA/PTX3 and the filtrate. Western blot analysis of HC1 (FIG. 15A), PTX3 (FIG. 15B), and PTX3 blot with additional DTT reduction (FIG. 15C) was conducted using unfiltered AME (as the control), a recovered sample (FIG. 15D) and a filtrate (FIG. 15E). After filtration, samples containing HC-HA/PTX3 recovered by water from the filter and filtrate were added at 12.5, 25, and 50 mg/mL of HA in the NO assay in comparison to 20 mg/mL of hyaluronic acid (HA) of IRM (FIG. 15D), *P<0.05, compared to the positive control).

### DETAILED DESCRIPTION

The placenta which comprises fetal tissues contains various critical biological properties that allow the fetus to develop normally. One such key biological component in placental tissue is the Heavy chain-hyaluronan/pentraxin 3 (HC-HA/PTX3) macromolecule. HC-HA/PTX3 can be found in fetal tissues such as placental amniotic membrane, amniotic membrane, etcetera, has anti-inflammatory, anti-scarring, regenerative properties, and/or wound healing properties. Disclosed herein in some embodiments are compositions, methods, kits, and devices comprising a fetal tissue product comprising a birth tissue or fetal tissue for use to prevent or treat eye indications. In some embodiments, the birth tissue or fetal tissue comprises a placental tissue. In some embodiments, a birth tissue or fetal tissue comprises an amniotic membrane tissue e.g., a placental amniotic membrane (PAM). In some embodiments, a fetal tissue comprises an amniotic membrane. In some embodiments, a fetal tissue comprises an amniotic membrane (AM) extract. In some embodiments, a fetal tissue may not comprise amniotic membrane fluid. In some embodiments, a fetal tissue product can be an amniotic membrane (AM) tissue product.

In some embodiments, disclosed herein is a composition comprising a fetal tissue product or amniotic membrane product. One example of an AM product described herein is a CAM360® AmnioGraft™ product which is also referred to as CAM360 AG, or CAM360® AmnioGraft™, or CAM360® AG™ product. These compositions can be processed by a sterilization manufacturing procedure thereby providing a product with surface adhesive properties. For example, a fetal tissue product provided herein comprises an adhesive property that allows application of the product on a surface of an eye without suturing, without any additional tissue adhesives, or any ocular glue. In some embodiments, a fetal tissue product disclosed herein can be applied directly on an ocular surface of an eye. In some embodiments, a fetal tissue product disclosed herein can also be applied on a surface of a contact lens, e.g., a lotrafilcon A surface prior to placing the contact lens on an ocular surface of an eye. In some embodiments, an increased adhesiveness of a fetal tissue product or AM product disclosed arises from a significantly reduced total hyaluronic acid (HA) content compared to other fetal tissue products, providing the advantages further described herein. In some embodiments, a fetal tissue product disclosed herein comprises a reduced total HA content of about 0.5, 0.4, 0.3, 0.25, 0.2 μg or less. In some embodiments, a fetal tissue product disclosed herein comprises a reduced total HA content of about 0.2 μg or less. In some embodiments, a fetal tissue product disclosed herein comprises a reduced total HA content of about 0.16 μg or less. Disclosed herein in some embodiments is a fetal tissue product comprising less than about 30 micrograms of hyaluronic acid (HA) per 32 cm². In some embodiments a fetal tissue product comprises less than about 20 micrograms of hyaluronic acid (HA) per 32 cm². In some embodiments, a fetal tissue product disclosed herein comprises notable reduction of the molecular weight of HA. In some embodiments, a fetal tissue product disclosed herein comprises about less than 500 kDa of total HA protein. In some embodiments, an increased adhesiveness of a fetal tissue product or AM product disclosed facilitates lubrication when applied on a surface e.g., an ocular surface or on an artificial surface such as a surface of a contact lens. In some embodiments, a fetal tissue product disclosed comprises a circular, square, rectangular structure. In some embodiments, a fetal tissue product disclosed does not comprise a particular structure e.g., an irregular shaped tissue product. In some embodiments, a fetal tissue product disclosed comprises a sheet or sheet product. In some embodiments, a sheet or sheet product disclosed comprises a circular shape. In some embodiments, a circular shaped fetal tissue product disclosed comprises a diameter from about 8-millimeter to 16 millimeter (16 mm). In some embodiments, a fetal tissue product disclosed comprises cryopreserved fetal tissue or fetal tissue product. In some embodiments, a fetal tissue product disclosed comprises a backing material that is gridded. In some embodiments, a backing material comprises a polyethersulfone (PES) polymer.

Disclosed herein in some embodiments is a medical device. In some embodiments, a medical device comprises an ophthalmic device. In some embodiments, an ophthalmic device comprises a fetal tissue product, AM product, cryopreserved AM disclosed herein. In some embodiments, an ophthalmic device is inserted into an ocular surface to mitigate one or more ocular conditions or diseases of an eye.

The compositions, methods, kits, devices (e.g., tissue grafts and tissue-derived biodegradable compositions) disclosed herein (e.g., fetal tissue product/extract or amniotic membrane product) can be used for their healing properties in promoting the prevention, repair, regeneration, healing, or treatment of an eye indication. In some embodiments, provided herein are methods of treating one or more ocular condition or disease. In some embodiments, provided herein are methods of treating two or more, three or more, four or more ocular conditions or diseases in an individual in need thereof. In some embodiments, methods provided treat an eye indication. In some embodiments, an eye indication can comprise a dry-eye-disease (DED) or condition. In some embodiments, an eye indication can comprise inflammation on the surface of an eye. In some embodiments, an eye indication can comprise a conjunctiva indication, a corneal indication or an eye disease or disorder that can occur on the surface of the eye. In some embodiments, a corneal indication can include a corneal disorder, corneal condition, or corneal infection. In certain embodiments, the corneal disorder, condition, or infection can be a corneal ulcer. In some embodiments, an ocular condition or disease comprises a corneal abrasion. In some embodiments, an ocular condition or disease comprises a corneal epithelial defect. In some embodiments, an ocular condition or disease comprises a corneal epithelial erosion. The corneal ulcer can be infectious. In certain embodiments, the corneal ulcer is not infectious. In some embodiments, an ocular condition or disease comprises macular hole. In some embodiments, an ocular condition or disease comprises superficial punctate keratitis. In some embodiments, an ocular condition or disease comprises non-healing injury/wound. In some embodiments, an ocular condition or disease comprises post debridement. In some embodiments, an ocular condition or disease comprises. In some embodiments, an ocular condition or disease comprises an epithelial basement membrane dystrophy. In some embodiments, an ocular condition or disease comprises neurotrophic keratitis. In some embodiments, an ocular condition or disease comprises a corneal epithelial defect. In some embodiments, an ocular condition or disease comprises a recurrent corneal epithelial erosion. In certain embodiments, the present disclosure can be used to promote prevention, repair, healing, or treatment on the surface of an eye. In a non-limiting example, the compositions, ophthalmic devices, methods, and/or kits of disclosed may be used for the prevention, repair, healing, or treatment on a surface of an eye, including a lens or a cornea. In some embodiments, the present disclosure can promote prevention, healing, or repair of an ocular condition following injury, trauma, disorder, condition, disease, or infection. The present disclosure can be used on a surface of an eye before or after an operation on an eye. For example, the present disclosure can be applied on a surface of an eye following ocular transplantation, injury, trauma, etcetera, to promote tissue healing and regeneration. In certain embodiments, administration of the present disclosure can be targeted to one, two, three or more conditions, infections, symptoms, diseases, syndromes in a subject in need thereof.

Disclosed herein in some embodiments is a method of making a fetal tissue product or AM product of the present disclosure. In some embodiments, a method of making a fetal tissue product comprises obtaining a fetal tissue, subjecting a fetal tissue to terminal sterilization and recovering a fetal tissue. In some embodiments, terminal sterilization comprises any one of the methods disclosed herein. In some embodiments, a fetal tissue is subjected to conditions sufficient to isolate a fetal tissue product disclosed herein.

Disclosed herein in some embodiments is a kit comprises a fetal tissue of the present disclosure for treating one or more disease or conditions of an eye. In some embodiments, a kit for comprises a fetal tissue product disclosed and a substrate. In some embodiments, a fetal tissue product comprises saline or buffer, so it is not a dehydrated fetal tissue product. In some embodiments, a buffer may be a physiological buffer e.g., any such buffer known to a person of skill in the art, or a skilled artisan. In some embodiments, a fetal tissue product is hydrated. In some embodiments, a substrate comprises an artificial contact lens. In some embodiments, an artificial contact lens comprises a soft lens or any other contact lens used on a surface of an eye.

The present disclosure provides a fetal tissue product/extract of various forms. For example, the fetal tissue product can be a component of an implantable device. In some embodiments, a device may be a biodegradable device. The fetal tissue product can be a kit comprising a fetal tissue product. In some embodiments, a fetal tissue can comprise an extract. In some embodiments, described herein, are methods of using a fetal tissue product for the treatment of an individual in need thereof. In some embodiments, a fetal tissue product can be a sheet comprising fetal tissue presented in any form that is minimally manipulated.

The fetal tissue product of the present disclosure may comprise amniotic membrane (AM) placental amniotic membrane (PAM), placental tissue, placenta, and/or a combination of any of these. In certain embodiments, the fetal tissue does not comprise umbilical cord.

In some embodiments, the many bioactive factors in fetal tissue are accessible from processed fetal tissue products. For example, and without limitations, fetal tissue e.g., amniotic fluid/amniotic membrane extracellular matrices contain several innate biological factors that can be isolated, processed into products with high concentrations of bioactive factors. Disclosed herein are processed fetal tissue products containing high concentrations of bioactive factors that can be useful for several purposes, including reducing ocular surface inflammation and scarring to promote ocular repair, ocular regenerative wound healing. Biological factors that may be found in fetal tissue may contain components such as extracellular matrices, growth factors, and cytokines.

### Certain Definitions

In this description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

Unless defined otherwise, all technical and scientific terms used herein have the same meaning as is commonly understood by one of skill in the art to which the claimed subject matter belongs.

As used herein, ranges and amounts are expressed as “about” a particular value or range. About also includes the exact amount. Hence “about 5 μg” means “about 5 μg” and also “5 μg.” The term “about” includes an amount that would be expected to be within 10% of the stated amount.

In some embodiments, the “fetal tissue” disclosed herein can comprise birth tissue. For example, the fetal tissue disclosed herein may comprise birth tissue from a mammal, e.g., an animal. For example, and without limitations, the birth tissue may be from an animal such as, for example, a non-human primate, a human, a cow, or a pig. In some embodiments, the birth tissue can be from an animal such as a human being. As used herein, “human tissue” means any tissue derived from a human body. In some embodiments, the human tissue is a fetal tissue selected from the group consisting of placental amniotic membrane, amniotic membrane, placenta, or any combination thereof. In some embodiments, birth tissue can comprise tissues that support fetal development (birth tissue is hereinafter also referred to as fetal tissue).

As used herein, “fetal tissue” means any isolated tissue derived from tissue used to support the development of a fetus. Examples of fetal tissue include, but are not limited to, (i) placental amniotic membrane (PAM), amniotic membrane, or substantially isolated PAM, or any combinations thereof. In some embodiments, a fetal tissue is selected from a group consisting of placental amniotic membrane (PAM), amniotic membrane, and combinations thereof. In some embodiments, a fetal tissue comprises amniotic membrane. Fetal tissue includes any form of the fetal tissue, including cryopreserved, terminally sterilized, lyophilized fetal tissue. In some embodiments, a fetal tissue is a graft, a sheet, or an extract. A “fetal tissue product” means any isolated tissue product disclosed that is isolated from fetal tissue. Non limiting examples of fetal tissue products include amniotic membrane product (AM product), placental amniotic membrane product (PAM product), fetal tissue device product, a fetal tissue product that is a terminally sterilized product, also includes a cryopreserved amniotic membrane (CAM) or amniograft (AG) product.

As used herein, “placenta” refers to an organ that connects a developing fetus to a maternal uterine wall to allow nutrient uptake, waste elimination, and gas exchange via the maternal blood supply. The placenta is composed of three layers. The innermost placental layer surrounding a fetus is called amnion. The allantois is the middle layer of the placenta (derived from the embryonic hindgut); blood vessels originating from the umbilicus traverse this membrane. The outermost layer of the placenta, the chorion, comes into contact with the endometrium. The chorion and allantois fuse to form the chorioallantoic membrane.

As used herein, “placental amniotic membrane” (PAM) refers to amniotic membrane derived from the placenta. In some embodiments, the PAM is substantially isolated. In some embodiments, placental amniotic membrane is free or substantially free from umbilical cord or umbilical cord amniotic membrane. In some embodiments, the fetal tissue products consist essentially of PAM.

As used herein, “human tissue” means any tissue derived from a human body. The human tissue is a fetal tissue selected from a group consisting of placental amniotic membrane, umbilical cord, umbilical cord amniotic membrane, chorion, amnion-chorion, placenta, or any combination thereof.

As used herein, “adhesiveness” of a fetal tissue product as disclosed refers to a property of a tissue or tissue product to stick to another surface, substrate, or material. Such a surface, substrate, or material may be an ocular surface of an eye e.g., a corneal surface of an eye, or a surface of a contact lens, e.g., an artificial contact lens. Adhesiveness may refer to the “stickiness” or the force that resists separation of two or more attached surfaces. Adhesiveness may refer to the ability of one substance to stick to or attach to another substance. Substances that stick to or attach to one another can resist separation, in this case, a fetal tissue product or AM product disclosed can be sticky, have adhesive property or stickiness property. The fetal tissue product disclosed may have adhesive property such that an adhesive bond or adhesiveness is increased or strong, or necessarily present.

As used herein, “lubrication” of a fetal tissue product as disclosed refers to the control of friction and wear by the introduction of a friction-reducing substance e.g., a film or layer of shear-accommodating lubricant between surfaces in contact so that wear, heat, or friction is reduced between these surfaces. The lubricant used can be a fluid, solid, or plastic substance that lubricates to reduce wear or friction.

As used herein, “minimal manipulation” means (1) for structural tissue, processing that does not alter an original, relevant characteristics of a tissue relating to the tissue's utility for reconstruction, repair, or replacement; and (2) for cells or nonstructural tissues, processing that does not alter relevant biological characteristics of cells or tissues.

Fetal tissue disclosed herein may comprise fetal tissue that comprise minimal manipulation. For example, minimal manipulation can include any form of the fetal tissue, including fresh, sheet form, cryopreserved, terminally sterilized, lyophilized fetal tissue. In some embodiments, fetal tissue may be processed to devascularize, devitalize, cleanse, lyophilize, terminally sterilize e.g., via gamma irradiation (with sterility assurance level, SAL, e.g., of 10-6) or electron beam sterilization. In some embodiments, the fetal tissue product may be a sheet or a graft form of an AM product. In some embodiments, the fetal tissue product may be a sheet, or a strip AM product of various shapes (circular, rectangular, square etcetera or any other different size).

The term “fresh fetal tissue” refers to fetal tissue that is less than 10 days old following birth, and which is in substantially the same form as it was following birth. In some embodiments, the fresh fetal tissue comprises fetal tissue cells. In some embodiments, the fetal tissue cells comprise pericytes. In some embodiments, at least 50%, at least 60%, at least 70%, at least 80%, at least 90%, or at least 95% of the biological activity of the cell tissue cells is maintained.

“Substantially isolated” or “isolated” when used in the context of a fetal tissue means that the fetal tissue is separated from most other non-fetal tissue materials (e.g., other tissues, red blood cells, veins, arteries) derived from the original source organism.

As used herein, the phrase “wherein the biological and structural integrity of the isolated fetal tissue is substantially preserved” means that when compared to the biological activity and structural integrity of fresh fetal tissue, the biological activity and structural integrity of the isolated fetal tissue has only decreased by about 5%, about 10%, about 15%, about 20%, or about 25% or less.

As used herein, “processing” means any activity performed on a fetal tissue or a preparation comprising a complex of macro-molecules comprising HC-HA/PTX3, other than recovery, donor screening, donor testing, storage, labeling, packaging, or distribution, such as testing for microorganisms, preparation, cutting, wash, sterilization, steps to inactivate or remove adventitious agents, preservation for storage, and removal from storage.

As used herein, the terms “isolated” can also mean a material (e.g., HC-HA/PTX3) substantially or essentially free from components that normally accompany it in its native state. In some embodiments, “isolated” means a material (e.g., HC-HA/PTX3) is about 50% or more free from components that normally accompany it in its native state, for example, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% free from components that normally accompany it in its native state.

As used herein, “biological activity” means the activity of biological macromolecules, such as polypeptides and polysaccharides of the fetal tissue including but not limited to HC-HA/PTX3. In some embodiments, the biological activity of polypeptides and polysaccharides found in the fetal tissue product is anti-inflammatory or anti-scarring. In some embodiments, the biological activity refers to the in vivo activities of HC-HA/PTX3 in the fetal tissue support product or physiological responses that result upon in vivo administration of the fetal tissue. In some embodiments, the biological activity of HC-HA/PTX3 in the fetal tissue is substantially preserved. In some embodiments, the activity of polypeptides and polysaccharides found in the fetal tissue product is promoting wound healing. In some embodiments, the activity of polypeptides and polysaccharides found in the fetal tissue is preventing scarring. In some embodiments, the activity of polypeptides and polysaccharides found in the fetal tissue is reducing inflammation. Biological activity, thus, encompasses therapeutic effects and pharmaceutical activity of HC-HA/PTX3 in the fetal tissue.

As used herein, “structural integrity” means the integrity of stroma and basement membrane that make up the fetal tissue. In some embodiments, the structural integrity of the fetal tissue results in suture pull out strength.

Disclosed herein are processed fetal tissue products containing high concentrations of bioactive factors that can be useful for various purposes, including reducing inflammation and scarring to promote regenerative wound healing. Biological factors that may be found in fetal tissue may contain components such as extracellular matrices, growth factors, and cytokines. Among extracellular matrices, hyaluronic acid (HA) and HA-containing complex, i.e., HC-HA/PTX3 that is uniquely and abundantly present in amniotic membrane (AM) umbilical cord amniotic membrane, and umbilical cord (UC) matrix. The HC-HA/PTX3—high molecular weight (HMW), or low-molecular weight hyaluronan (HA) covalently linked with heavy chain (HC) 1 from inter-α-trypsin inhibitor and further complexed with pentraxin3 (PTX3)—is one key active component of isolated placenta, placental amniotic membrane (PAM), amniotic membrane (AM), and/or a combination of these, that is responsible for the aforementioned effects to promote ocular regenerative, repair, tissue, or wound healing.

In some embodiments HC-HA/PTX3 is isolated from amniotic membrane. In some embodiments, the substantially isolated HC-HA is derived from placenta, placental amniotic membrane (PAM), amniotic membrane (AM), umbilical cord (UC), umbilical cord amniotic membrane (UCAM) or a combination thereof. In some embodiments, the substantially HC-HA/PTX3 is derived from frozen or previously frozen placental amniotic membrane (PAM), frozen or previously frozen amniotic membrane (AM), frozen or previously frozen umbilical cord (UC), frozen or previously frozen umbilical cord amniotic membrane (UCAM), frozen or previously frozen placenta, or a combination thereof.

As used herein, “hyaluronan,” “hyaluronic acid,” or “hyaluronate” (HA) are used interchangeably to refer to a substantially non-sulfated linear glycosaminoglycan (GAG) with repeating disaccharide units of D-glucuronic acid and N-acetylglucosamine (D-glucuronosyl-N-acetylglucosamine).

As used herein, the term “low molecular weight” or “LMW,” as in low molecular weight hyaluronan (LMW HA), is meant to refer to HA that has a weight average molecular weight that is less than 600 kDa less than 500 kDa, such as for example, less than about 400 kDa, less than about 300 kDa, less than about 200 kDa, less than about 100 kDa, less than about 50 kDa, less than about 40 kDa, less than about 30 kDa, less than about 20 kDa, about 200-300 kDa, about 1-300 kDa, about 15 to about 40 kDa, or about 8-10 kDa.

As used herein, pentraxin 3, or PTX3, protein or polypeptide refers to any PTX3 protein, including but not limited to, a native PTX3 protein, and a PTX3 protein extracted from cells or tissues. PTX3 include multimeric forms (e.g., homomultimer) of PTX3, including, but not limited to, dimeric, trimeric, tetrameric, pentameric, hexameric, tetrameric, octameric, and other multimeric forms naturally produced.

As used herein, tumor necrosis factor stimulated gene-6 (TSG-6) refers to any TSG-6 protein or polypeptide, including but not limited to, a native TSG-6 protein, and a TSG-6 protein extracted from cells or tissues.

As used herein, inter-α-inhibitor (IαI) refers to the IαI protein comprised of light chain (i.e., bikunin) and one or both heavy chains of type HC1 or HC2 covalently connected by a chondroitin sulfate chain. In some embodiments, the source of IαI is from serum or from cells producing IαI e.g., hepatic cells or amniotic epithelial or stromal cells or umbilical epithelial or stromal cells under a constitutive mode stimulation by proinflammatory cytokines such as IL-1 or TNF-α.

Accordingly, it is important to isolate and process fetal tissue that preserve the activity of biological factors, e.g., HC-HA/PTX3, HA, and/or other proteins of interest.

In some embodiments, the fetal tissue product disclosed herein comprises HC-HA/PTX3. In some embodiments, fetal tissue product comprising HC-HA/PTX3 can be useful for the treatment of eye indications. For example, the fetal tissue product may comprise a complex of native HC-HA/PTX3 as disclosed herein.

Disclosed herein are methods, kits, compositions, devices, processes comprising fetal tissue products/extracts for use in prevention or a treatment of ocular disorders, ocular pain, ocular disease, or conditions of the eye e.g., on the surface of an eye such as a corneal, conjunctiva, etcetera. In certain embodiments, treatment can comprise administering to the eye of the individual or subject with the disclosed fetal tissue product, thereby treating the eye disorder, or indication affecting the eye.

As used herein, the terms “treat,” “treating” or “treatment,” and other grammatical equivalents, include alleviating, abating or ameliorating one or more symptoms of a disorder, a disease, symptom or condition, ameliorating, preventing or reducing the appearance, severity or frequency of one or more additional symptoms of a disorder, disease, symptom, or condition, ameliorating or preventing the underlying causes of one or more symptoms of a disorder, disease, symptom, or condition, inhibiting the disease or condition, such as, for example, arresting the development of the disorder, disease, symptom, or condition, relieving the disease or condition, causing regression of the disorder, disease, symptom, or condition, relieving a condition caused by the disorder, disease, symptom, or condition, or inhibiting the symptoms of the disorder, disease, symptom, or condition either prophylactically and/or therapeutically. In a non-limiting example, for prophylactic benefit, the composition or fetal tissue product can comprise an isolate HC-HA/PTX3 complex. The composition disclosed herein can be administered to an individual at risk of developing a particular disorder of the eye, predisposed to developing a particular disorder of the eye, or to an individual reporting one or more of the physiological symptoms of a disorder or disease of the eye.

As used herein, the terms “subject,” “individual” and “patient” are used interchangeably. None of the terms are to be interpreted as requiring the supervision of a medical professional (e.g., a doctor, nurse, physician's assistant, orderly, hospice worker). As used herein, a subject is any animal, including mammals (e.g., a human or non-human animal) and non-mammals. In one embodiment of the methods and compositions provided herein, a mammal is a human. As disclosed herein, the present disclosure are for use in treatment of an ocular disorder, a conditions, in an individual in need of treatment comprising administering to the ocular surface of an individual or a subject with an effective amount of a fetal tissue product, thereby treating the ocular indication e.g., ocular disorder, condition, disease, etcetera.

As used herein the terms “effective amount” or “therapeutically effective amount,” as used herein, refer to enough or sufficient amount of an agent or a compound being administered on an ocular surface, for example, which will relieve to some extent one or more of the symptoms of an ocular disease or condition being treated. In some embodiments, the result is a reduction and/or alleviation of the signs, symptoms, or causes of a disease, or any other desired alteration of a biological system. For example, an “effective amount” for therapeutic uses is an amount of the composition including a compound as disclosed herein required to provide a clinically significant decrease in disease symptoms without undue adverse side effects. In some embodiments, an appropriate “effective amount” in any individual case is determined using techniques, such as a dose escalation study.

The term “therapeutically effective amount” includes, for example, a prophylactically effective amount. An “effective amount” of a compound disclosed herein, is an amount effective to achieve a desired effect or therapeutic improvement without undue adverse side effects. It is understood that in some embodiments, “an effective amount” or “a therapeutically effective amount” varies from subject to subject, due to variation in metabolism of a composition, age, weight, general condition of a subject, the condition being treated, the severity of the condition being treated, and the judgment of the prescribing physician. In some embodiments, an effective amount is an amount of a product or compound sufficient to promote treatment (as defined herein) of an ocular disorder, a disease, or a condition. Disclosed herein are various forms of fetal tissue which may be further processed to access bioactive factors for therapeutics as disclosed herein. The fetal tissue is obtained from donors and septically processed.

In some embodiments, a repair, prevention, or treatment may extend to indications or complications arising from a condition, disorder, or disease of the eye.

The AM compositions of the present disclosure may be useful as or in conjunction with a medical device for use to prevent, repair, or treat an ocular disorder, condition, or disease. In some embodiments, the fetal tissue product disclosed herein can comprise a device such as a graft, an allograft, a sheet, a strip, or a combination thereof. A fetal tissue product of the present disclosure may be applied on a contact lens, e.g., a collagen shield, or a collagen corneal shield. In some embodiments, the lens can comprise a soft contact lens, or a rigid gas-permeable (RGP) lens. In some embodiments, a contact lens may comprise an extended-wear lens, an extended-wear disposable lens, a planned replacement lens, or any combination thereof. In some embodiments, a contact lens may be a soft or hydrophilic lens. RGP lenses may contain silicone material which may allow oxygen to pass through the lens materials to the eye. Non-limiting examples of a RGP contact lenses providers include lenses from Alcon, Unilens Vision, Bausch & Lomb, CIBA Vision, CooperVision and Johnson & Johnson. In some embodiments, the device from the fetal tissue may be a strip, a patch, a tube, a sheet comprising a tubular structure, with annular opening. The device comprising the fetal tissue product can comprise any shape: circular, square, spherical, tubular, rectangular, etcetera, as needed for use in an eye indication. In some embodiments, the bioactive factors disclosed herein can be useful for the treatment of ocular conditions, disorders, or diseases.

In some embodiments, the amniotic membrane preparations and amniotic membrane products retain biological activity or comprise bioactive factors that can be useful in the repair of an ocular disorder or disease. In some embodiments, the bioactive factors in processed fetal tissue and fetal tissue products/extracts can be useful in the regeneration of the ocular tissues. For example, any without limitation, the bioactive factors in processed fetal tissue and fetal tissue products can prevent, repair, heal or regenerate an ocular condition, disorder, or disease e.g., an ocular cancer, an ocular pain, an ocular wound, an ocular swelling, an ocular inflammation or an ocular infection, an ocular scarring, an ocular trauma, an injury, ocular dryness, a condition of dry eye disease, and/or a combination thereof.

The composition, methods, kits, devices (e.g., ocular grafts, sheet, strips, or other birth tissue-derived biodegradable devices) compositions disclosed herein (e.g., fetal tissue product/extract) can be used to promote repair, regeneration, healing, treatment to any ocular indication. In some embodiments, the present disclosure can be used to promote ocular tissue repair. In some embodiments, promoting ocular tissue repair can include proliferation, remodeling, or reconstruction of an ocular tissue following trauma, injury or due to any eye indication (condition, disorder, disease, infection etcetera).

In certain embodiments, disclosed herein, are fetal tissue products of the present disclosure useful for the treatment of any number of ocular disorders, e.g., ocular immune-modulatory disorder or disease or any infectious disease affecting an eye of a subject e.g., an eye cancer.

### Fetal Tissue Products

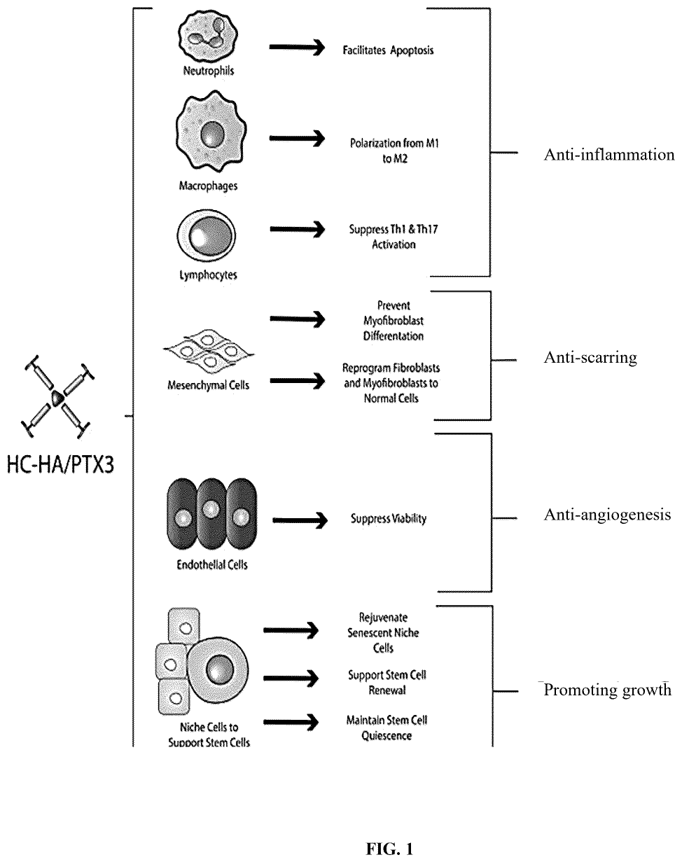

Disclosed herein in some embodiments is a fetal tissue product comprising heavy chain-hyaluronic acid/pentraxin 3 complex (HC-HA/PTX3). In some embodiments, HC-HA/PTX3 complex. In some embodiments, a fetal tissue product or amniotic membrane product disclosed contains potent biological properties comprising e.g., HC-HA/PTX3 complex. In some embodiments, potent biological properties are found in hydrated, cryopreserved amniotic membrane (AM) of the fetal tissue product or AM products. In some embodiments, potent biological properties in a fetal tissue product or AM tissue product may promote a body's healing mechanisms. Non-limiting examples of a body's healing mechanisms include, anti-inflammatory, anti-scarring, anti-angiogenic, or regenerative (stemness) healing mechanisms which have been recognized by the U.S. Food and Drug Administration (FDA). One such potent biological component within hydrated, cryopreserved fetal tissue product or AM disclosed comprises a heavy chain-hyaluronan/pentraxin 3 (HC-HA/PTX3) matrix, or complex that is formed by a covalent linkage between hyaluronic acid (HA) and heavy chain 1 (HC1) of inter-α-trypsin inhibitor that is further tightly associated with octameric pentraxin 3 (PTX3). In some embodiments, HC-HA/PTX3 may be a key component that provides biological properties of a purified fetal tissue product e.g., amniotic membrane tissue product. In some embodiments, HC-HA/PTX3 purified from cryopreserved AM has been shown to exert anti-inflammatory properties by promoting apoptosis of lipopolysaccharide (LPS)-, interferon (IFN)-γ, or LPS/IFN-γ-activated proinflammatory neutrophils and macrophages. The anti-inflammatory properties of hydrated cryopreserved AM are retained in the water-soluble AM extract (AME), indicating that the immunomodulatory properties of hydrated cryopreserved AM are not dependent on physical structure but rather the retention and preservation of HC-HA/PTX3 during the manufacturing processing. Anti-scarring: Suppressing inflammation can indirectly reduce scarring, and in addition HC-HA/PTX3 has been shown to directly reduce scarring by inhibiting the transforming growth factor (TGF)-β-mediated canonical signaling. Anti-angiogenic: HC-HA/PTX3 exerts an anti-angiogenic action by suppressing cell viability more significantly than HA or AM stromal extract and by inhibiting cell migration and tube formation of human umbilical vein endothelial cells. Regenerative healing or stemness of HC-HA/PTX3 has been shown to preserve quiescence and promote self-renewal of stem cells e.g., self-renewal of limbal epithelial stem cells (LESCs) through the upregulation of bone morphogenetic protein and non-canonical Wnt signaling in limbal niche cells (LNCs) and subsequent bone morphogenetic protein signaling in LESCs. Non-limiting exemplary biological mechanisms promoted by HC-HA/PTX3 in regenerative wound healing are summarized in FIG. 1.

Disclosed herein in some embodiments, a fetal tissue may comprise amniotic membrane, optionally, placental amniotic membrane excluding umbilical cord amniotic membrane. Amniotic membrane (AM) contains several innate biological factors useful for several purposes, including wound healing and reducing inflammation and scarring. The extracellular matrix (ECM) of AM and are enriched in hyaluronic acid (HA) having a critical or key matrix component called, HC-HA/PTX3 (Heavy Chain 1-Hyaluronic Acid-Pentraxin 3; the “-” denotes covalent linkage and “/” denotes non-covalent tight binding within the biological healing substance. Native HC-HA/PTX3 is a key matrix component of both AM isolated from a tissue or a cell or an organism which exerts multiple therapeutically useful biologic actions of AM or placenta. Native HC-HA/PTX3 is isolated from the fetal tissue obtained from a human or animal. Disclosed herewith are fetal tissue products comprising HC-HA/PTX3. In some embodiments, ta fetal tissue product disclosed herein comprises a HC-HA/PTX3 complex. In some embodiments, an HC-HA/PTX3 or HC-HA/PTX3 complex comprises a low molecular weight (LMW) hyaluronic acid (HA). In some embodiments an HC-HA/PTX3 or HC-HA/PTX3 complex comprises a high molecular weight (HMW) hyaluronic acid (HA). In some embodiments, a hyaluronic acid molecular may be covalently linked with heavy chain (HC) 1 from inter-α-trypsin inhibitor and which may further be complexed with pentraxin3 (PTX3). In some embodiments, a high molecular weight HA molecule may be covalently linked with heavy chain (HC) 1 from inter-α-trypsin inhibitor and which may further be complexed with pentraxin3 (PTX3). In some embodiments, a low molecular weight HA molecule may be covalently linked with heavy chain (HC) 1 from inter-α-trypsin inhibitor and which may further be complexed with pentraxin3 (PTX3) Disclosed herein is a fetal tissue product cryopreserved and terminally sterilized to preserve HC-HA/PTX3 but reduce the amount of HA for use in preventing, repairing, or healing of eye indications. As opposed to dry terminal sterilization the wet terminal sterilization described herein preserves HC-HA/PTX3 activity.

Disclosed herein in some embodiments is a composition comprising a fetal tissue product or an amniotic membrane (AM) product optionally, placental amniotic membrane excluding umbilical cord amniotic membrane. In some embodiments, a fetal tissue product disclosed comprises a sheet e.g., an AM sheet product. In some embodiments, an AM product comprises cryopreserved AM product (CAM). In some embodiments, a fetal tissue product or amniotic membrane product comprises less than about 30 micrograms of hyaluronic acid (HA) per 32 cm². In some embodiments, a fetal tissue product disclosed comprises less than about 20 micrograms of hyaluronic acid per 32 cm². In some embodiments, a fetal tissue product disclosed comprises less than about 10 micrograms (μm) of hyaluronic acid per 32 cm2. In some embodiments, a fetal tissue product disclosed comprises about 10 μm, 9 μm, 8 μm, 7 μm, 6 μm, 5 μm, 4 μm, 3 μm, 2 micrograms (μm) of hyaluronic acid per 32 cm². In some embodiments, a fetal tissue product disclosed comprises from about 1 microgram to about 30 micrograms of hyaluronic acid per 32 cm². In some embodiments, a fetal tissue product disclosed comprises from about 2 micrograms to about 30 micrograms of hyaluronic acid per 32 cm². In some embodiments, a fetal tissue product disclosed comprises from about 5 micrograms to about 30 micrograms of hyaluronic acid per 32 cm². In some embodiments, a fetal tissue product disclosed comprises from about 1 microgram to about 20 micrograms of hyaluronic acid per 32 cm². In some embodiments, a fetal tissue product disclosed comprises from about 2 micrograms to about 20 micrograms of hyaluronic acid per 32 cm². In some embodiments, a fetal tissue product disclosed comprises from about 5 micrograms to about 20 micrograms of hyaluronic acid per 32 cm². In some embodiments, a fetal tissue product disclosed comprises from about 1 microgram to about 10 micrograms of hyaluronic acid per 32 cm². In some embodiments, a fetal tissue product disclosed comprises from about 2 micrograms to about 10 micrograms of hyaluronic acid per 32 cm². In some embodiments, a fetal tissue product disclosed comprises from about 5 micrograms to about 10 micrograms of hyaluronic acid per 32 cm². In some embodiments, a fetal tissue product disclosed comprises greater than 50 micrograms of hyaluronic acid (HA) per 32 cm². In some embodiments, a fetal tissue product disclosed comprises greater than 40 micrograms of hyaluronic acid (HA) per 32 cm². In some embodiments, a fetal tissue product disclosed comprises greater than 35 micrograms of hyaluronic acid (HA) per 32 cm². In some embodiments, a fetal tissue product disclosed comprises greater than 30 micrograms of hyaluronic acid (HA) per 32 cm². In some embodiments, a fetal tissue product disclosed comprises greater than 25 micrograms of hyaluronic acid (HA) per 32 cm². In some embodiments, a fetal tissue product disclosed comprises greater than 20 micrograms of hyaluronic acid (HA) per 32 cm². In some embodiments, a fetal tissue product disclosed comprises greater than 15 micrograms of hyaluronic acid (HA) per 32 cm². In some embodiments, a fetal tissue product disclosed comprises greater than 10 micrograms of hyaluronic acid (HA) per 32 cm². In some embodiments, a fetal tissue product disclosed comprises greater than 5 micrograms of hyaluronic acid (HA) per 32 cm². In some embodiments, a fetal tissue product disclosed comprises about 1, 2, 3, 4, 5, 10, 15, 20, 25, 30, 40, 45, or 50 micrograms of hyaluronic acid (HA) per 32 cm². In some embodiments, a fetal tissue product disclosed may comprise greater than 50 micrograms of hyaluronic acid (HA) per 32 cm².

In some embodiments, a fetal tissue product disclosed comprises a hyaluronic acid (HA) of low molecular weight HA and comprises a low content of HA. In some embodiments, a fetal tissue product disclosed may comprise a HA of low molecular weight HA, optionally wherein a low MW HA comprises an average size of 500 kD or less, 495 kDa or less, 480 kDa or less, 470 kDa or less. In some embodiments, a fetal tissue product disclosed may comprise a HA of low molecular weight HA, optionally wherein a low MW HA comprises an average size of about 450, 400, 350, 300, 250, 200, 150, 100, 50, 40, 30, 20 kDa or less. In some embodiments, a fetal tissue product disclosed comprises about 20% to about 90% of HA covalently bonded to a heavy chain 1 of IαI (HC1) complexed with pentraxin 3 (PTX3). In some embodiments, a fetal tissue product disclosed comprises about 30% to about 75% of HA is covalently bonded to a heavy chain 1 of IαI (HC1) complexed with pentraxin 3 (PTX3). In some embodiments, a fetal tissue product disclosed comprises about 20% to about 50% of HA covalently bonded to a heavy chain 1 of IαI (HC1 or HC) complexed with pentraxin 3 (PTX3). In some embodiments, a fetal tissue product disclosed comprises about 20% to about 40% of HA covalently bonded to a heavy chain 1 of IαI (HC1 or HC) complexed with pentraxin 3 (PTX3). In some embodiments, a fetal tissue product disclosed comprises circular-shaped AM tissue product. In some embodiments, a fetal tissue product disclosed does not comprise a circular-shaped AM tissue product, In some embodiments, an AM tissue product disclosed comprises a low total HA content comprising about less than 10 μg, about 9 μg, about 8 μg, about 7 μg, about 6 μg, about 5 μg, about 4 μg, about 3 μg, about 2 μg, about 1 μg or less. In some embodiments, an AM tissue product disclosed comprises a low total HA content comprising about 1 μg, about 0.9 μg, about 0.8 μg, about μg μg, about 0.6 μg, about 0.5 μg, about 0.4 μg, about 0.3 μg, about 0.2 μg, about 0.1 μg or less. In some embodiments, an AM tissue product disclosed comprises a low total HA content comprising less than 1 an AM tissue product disclosed comprises a low total HA content comprising about 1 μg. In some embodiments, an AM tissue product disclosed comprises a low total HA content comprising about 0.2 μg or less. In some embodiments, an AM tissue product disclosed comprises a low total HA content comprising about 0.16 μg or less.

Disclosed herein in some embodiments is a fetal tissue comprising a placental amniotic membrane (PAM) tissue, e.g., an amniotic membrane (PAM). In some embodiments, a fetal tissue product e.g., an AM tissue product described herein comprises a fetal tissue that was previously frozen. In some embodiments, a fetal tissue product, e.g., an AM product described herein comprises a fetal tissue that was not previously frozen. In some embodiments, a fetal tissue product described herein comprises a sheet. In some embodiments, a fetal tissue product described herein comprises a sheet that is circular. In some embodiments a fetal tissue product described herein comprises a sheet that is not a circular sheet e.g., an irregularly shaped or structured sheet. In some embodiments, a sheet of the present disclosure e.g., a circular sheet comprises a diameter from about 8 millimeters to 16 millimeters. In some embodiments, a sheet comprising a fetal tissue product of the present disclosure has a diameter of about 12 millimeters. In some embodiments, a sheet comprising a fetal tissue product of the present disclosure has an average thickness of about less than about 200 micrometers. In some embodiments, a sheet comprising a fetal tissue product of the present disclosure has an average thickness of between about 150 micrometers and about 75 micrometers. In some embodiments, a sheet comprising a fetal tissue product of the present disclosure has an average thickness of greater than about 50 micrometers. In some embodiments, a sheet comprising a fetal tissue product of the present disclosure has an average thickness of about 5, 6, 7, 8, 9, 10, 11, 12, 13, 14, 15, 16, 17, 18, 19, 20, 21, 22, 23, 24, 25, 26, 27, 28, 29, 30, 40 micrometers (μm) or greater. In some embodiments, a sheet comprising a fetal tissue product of the present disclosure has an average thickness of about 50 μm, 45 μm, 40 μm, 35 μm, 30 μm, 25 μm, 20 μm, 15 μm, or 10 μm, or less. In some embodiments, a sheet comprising a fetal tissue product of the present disclosure has an average thickness of greater than 5 μm. In some embodiments, a sheet comprising a fetal tissue product of the present disclosure has an average thickness of greater than 10 μm. In some embodiments, a sheet comprising a fetal tissue product of the present disclosure has an average thickness of greater than 15 μm. In some embodiments, a sheet comprising a fetal tissue product of the present disclosure has an average thickness of about 9 μm. In some embodiments, a sheet comprising a fetal tissue product of the present disclosure has an average thickness of about 12 μm. In some embodiments, a sheet comprising a fetal tissue product of the present disclosure has an average thickness of about 20 μm. In some embodiments, a sheet comprising a fetal tissue product of the present disclosure has an average thickness of about 25 μm. In some embodiments, a sheet comprising a fetal tissue product of the present disclosure has an average thickness of less than 50, 45, 40, 35, or 25 μm or less. In some embodiments, a sheet comprising a fetal tissue product of the present disclosure has an average thickness of about 20 μm. In some embodiments, a sheet comprising a fetal tissue product of the present disclosure has an average thickness of about 8 μm to about 13 μm. In some embodiments, a sheet comprising a fetal tissue product of the present disclosure has an average thickness of about 6 μm to about 18 μm. In some embodiments, a sheet comprising a fetal tissue product of the present disclosure has an average thickness of about 10 μm to about 20 μm. In some embodiments, a sheet comprising a fetal tissue product of the present disclosure has an average thickness of about 15 μm to about 25 μm. In some embodiments, a sheet comprising a fetal tissue product of the present disclosure has an average thickness of about 20 μm to 30 μm. In some embodiments, a sheet comprising a fetal tissue product of the present disclosure has an average thickness of between 8 μm to 15 μm. In some embodiments, a sheet comprising a fetal tissue product of the present disclosure has an average thickness between 12 μm to 20 μm. In some embodiments, a sheet comprising a fetal tissue product of the present disclosure has an average thickness between 15 μm to 30 μm. In some embodiments, a sheet comprising a fetal tissue product of the present disclosure has an average thickness of less 30 μm. In some embodiments, a sheet comprising a fetal tissue product of the present disclosure has an average thickness less than 25 μm. In some embodiments, a sheet comprising a fetal tissue product of the present disclosure has an average thickness of about 20 μm or less. In some embodiments, a sheet comprising a fetal tissue product of the present disclosure has an average thickness of about 15 μm or less. In some embodiments, a sheet comprising a fetal tissue product of the present disclosure has an average thickness of about 5, 6, 7, 8, 9, 10, 11, 12, 13, 14, 15, 16, 17, 18, 19, 20, 21, 22, 23, 24, 25, 26, 27, 28, 29, 30, 40 micrometers (μm). In some embodiments, a fetal tissue product disclosed comprises a sheet that is substantially flat. In some embodiments, a fetal tissue product comprises a hydrated tissue product that is not dehydrated. In some embodiments, a fetal tissue product disclosed comprises water that is not removed from a fetal tissue. In some embodiments, a fetal tissue product disclosed undergoes terminal sterilization. In some embodiments, terminal sterilization comprises gamma irradiation, UV irradiation, or electron beam sterilization. In some embodiments, a fetal tissue product disclosed terminal sterilization comprises gamma irradiation. In some embodiments, a fetal tissue product disclosed terminal sterilization comprises electron beam irradiation. In some embodiments, disclosed herein is a fetal tissue product where substantially all of the cells of the fetal tissue are dead. In some embodiments, a fetal tissue product disclosed terminally sterilized at a temperature at or below Oo C. In some embodiments, a fetal tissue product disclosed may be adhered to a backing material. In some embodiments, a backing material comprises any backing material disclosed herein e.g., a polyethersulfone (PES) polymer, a nylon, or a nitrocellulose membrane. In some embodiments, a backing material is gridded. In some embodiments, a fetal tissue product disclosed does not comprise a ring-shaped support structure. In some embodiments, a fetal tissue product disclosed may be a clear fetal tissue product. In some embodiments, a fetal tissue product disclosed may be optically clear. In some embodiments, a fetal tissue product disclosed may be packaged in normal (0.9%) saline. In some embodiments, a fetal tissue product disclosed may be stable at a temperature of 20° C. to 25° C. for at least one year. In some embodiments, a fetal tissue product disclosed adheres to a surface comprised of lotrafilcon A with a force of greater than 0.0001, 0.01, 1, 2, 5, 10, 15, or 20 pounds per square inch. In some embodiments, a fetal tissue product disclosed adheres to a surface comprised of lotrafilcon A with a force of greater than 20 pounds per square inch. In some embodiments, a fetal tissue product disclosed exhibits increased adhesion to a surface comprised of lotrafilcon A after sterilization with gamma irradiation at a temperature of below 0° C. In some embodiments, a fetal tissue product disclosed adhered to a contact lens such that when inserted into an eye of an individual a fetal tissue product contacts a surface of an eye of an individual. In some embodiments, a fetal tissue product disclosed has increased adhesiveness when placed on an ocular surface of an eye or an artificial contact lens. In some embodiments, a fetal tissue product disclosed an artificial contact lens comprises a soft contact lens. In some embodiments, a soft contact lens comprises a collagen corneal shield, or collagen shield. In some embodiments, a soft contact lens comprises a bandage contact lens. In certain embodiments, the fetal tissue or fetal tissue product is not lyophilized. In certain embodiments, the fetal tissue or fetal tissue product is not dehydrated or has not had water substantially removed during processing, packaging, or storage. In certain embodiments, the fetal tissue or fetal tissue product is not dehydrated or has not had water substantially removed when it is used as a therapeutic. In certain embodiments, the fetal tissue or fetal tissue product is not minced, morselized, micronized, or powdered in form.

### HA Content

Fetal tissue products of the current disclosure comprise low levels of hyaluronic acid (HA) which, without being bound by theory, is responsible for the highly adhesive character that increases suitably for indications treating the eye. In some embodiments, a fetal tissue product can comprise less than about 30 micrograms of HA per 32 cm². In some embodiments, a fetal tissue product can be a sheet. In some embodiments, a fetal tissue product can comprise less than about 20 micrograms of hyaluronic acid per 32 cm². In some embodiments, a fetal tissue product can comprise less than about 10 micrograms of hyaluronic acid per 32 cm². In some embodiments, a fetal tissue product can comprise about 1 micrograms of HA per 32 cm² to about 30 micrograms of HA per 32 cm². In some embodiments, a fetal tissue product can comprise about 1 micrograms of HA per 32 cm² to about 3 micrograms of HA per 32 cm², about 1 micrograms of HA per 32 cm² to about 5 micrograms of HA per 32 cm², about 1 micrograms of HA per 32 cm² to about 7 micrograms of HA per 32 cm², about 1 micrograms of HA per 32 cm² to about 9 micrograms of HA per 32 cm², about 1 micrograms of HA per 32 cm² to about 10 micrograms of HA per 32 cm², about 1 micrograms of HA per 32 cm2 to about 12 micrograms of HA per 32 cm2, about 1 micrograms of HA per 32 cm2 to about 15 micrograms of HA per 32 cm2, about 1 micrograms of HA per 32 cm² to about 20 micrograms of HA per 32 cm², about 1 micrograms of HA per 32 cm2 to about 25 micrograms of HA per 32 cm2, about 1 micrograms of HA per 32 cm² to about 28 micrograms of HA per 32 cm², about 1 micrograms of hyaluronic acid (HA) per 32 cm² to about 30 micrograms of HA per 32 cm2, about 3 micrograms of HA per 32 cm² to about 5 micrograms of hyaluronic acid (HA per 32 cm2, about 3 micrograms of HA per 32 cm² to about 7 micrograms of HA per 32 cm², about 3 micrograms of HA per 32 cm2 to about 9 micrograms of HA per 32 cm², about 3 micrograms of HA per 32 cm² to about 10 micrograms of HA per 32 cm², about 3 micrograms of HA per 32 cm² to about 12 micrograms of HA per 32 cm², about 3 micrograms of HA per 32 cm2 to about 15 micrograms of HA per 32 cm², about 3 micrograms of HA per 32 cm² to about 20 micrograms of HA per 32 cm2, about 3 micrograms of HA per 32 cm² to about 25 micrograms of hyaluronic acid (HA per 32 cm², about 3 micrograms of HA per 32 cm² to about 28 micrograms of HA per 32 cm2, about 3 micrograms of HA per 32 cm² to about 30 micrograms of HA per 32 cm², about 5 micrograms of HA per 32 cm² to about 7 micrograms of HA per 32 cm², about 5 micrograms of HA per 32 cm² to about 9 micrograms of HA per 32 cm², about 5 micrograms of HA per 32 cm2 to about 10 micrograms of HA per 32 cm², about 5 micrograms of HA per 32 cm² to about 12 micrograms of HA) per 32 cm², about 5 micrograms of HA per 32 cm² to about 15 micrograms of HA per 32 cm², about 5 micrograms of HA per 32 cm² to about 20 micrograms of HA per 32 cm², about 5 micrograms of HA per 32 cm² to about 25 micrograms of HA per 32 cm², about 5 micrograms of HA per 32 cm² to about 28 micrograms of HA per 32 cm², about 5 micrograms of HA per 32 cm² to about 30 micrograms of HA per 32 cm², about 7 micrograms of HA per 32 cm² to about 9 micrograms of hyaluronic acid (HA) per 32 cm², about 7 micrograms of hyaluronic acid (HA) per 32 cm² to about 10 micrograms of hyaluronic acid (HA) per 32 cm², about 7 micrograms of hyaluronic acid (HA) per 32 cm² to about 12 micrograms of hyaluronic acid (HA) per 32 cm2, about 7 micrograms of hyaluronic acid (HA) per 32 cm² to about 15 micrograms of hyaluronic acid (HA) per 32 cm², about 7 micrograms of hyaluronic acid (HA) per 32 cm² to about 20 micrograms of hyaluronic acid (HA) per 32 cm2, about 7 micrograms of hyaluronic acid (HA) per 32 cm² to about 25 micrograms of hyaluronic acid (HA) per 32 cm², about 7 micrograms of hyaluronic acid (HA) per 32 cm2 to about 28 micrograms of hyaluronic acid (HA) per 32 cm², about 7 micrograms of hyaluronic acid (HA) per 32 cm2 to about 30 micrograms of hyaluronic acid (HA) per 32 cm², about 9 micrograms of hyaluronic acid (HA) per 32 cm2 to about 10 micrograms of hyaluronic acid (HA) per 32 cm², about 9 micrograms of hyaluronic acid (HA) per 32 cm² to about 12 micrograms of hyaluronic acid (HA) per 32 cm2, about 9 micrograms of hyaluronic acid (HA) per 32 cm² to about 15 micrograms of hyaluronic acid (HA) per 32 cm², about 9 micrograms of hyaluronic acid (HA) per 32 cm² to about 20 micrograms of hyaluronic acid (HA) per 32 cm², about 9 micrograms of hyaluronic acid (HA) per 32 cm2 to about 25 micrograms of hyaluronic acid (HA) per 32 cm2, about 9 micrograms of hyaluronic acid (HA) per 32 cm2 to about 28 micrograms of hyaluronic acid (HA) per 32 cm2, about 9 micrograms of hyaluronic acid (HA) per 32 cm2 to about 30 micrograms of hyaluronic acid (HA) per 32 cm², about 10 micrograms of hyaluronic acid (HA) per 32 cm² to about 12 micrograms of hyaluronic acid (HA) per 32 cm2, about 10 micrograms of hyaluronic acid (HA) per 32 cm² to about 15 micrograms of hyaluronic acid (HA) per 32 cm², about 10 micrograms of hyaluronic acid (HA) per 32 cm² to about 20 micrograms of hyaluronic acid (HA) per 32 cm2, about 10 micrograms of hyaluronic acid (HA) per 32 cm² to about 25 micrograms of hyaluronic acid (HA) per 32 cm², about 10 micrograms of hyaluronic acid (HA) per 32 cm² to about 28 micrograms of hyaluronic acid (HA) per 32 cm², about 10 micrograms of hyaluronic acid (HA) per 32 cm² to about 30 micrograms of hyaluronic acid (HA) per 32 cm², about 12 micrograms of hyaluronic acid (HA) per 32 cm² to about 15 micrograms of hyaluronic acid (HA) per 32 cm2, about 12 micrograms of hyaluronic acid (HA) per 32 cm² to about 20 micrograms of hyaluronic acid (HA) per 32 cm², about 12 micrograms of hyaluronic acid (HA) per 32 cm² to about 25 micrograms of hyaluronic acid (HA) per 32 cm2, about 12 micrograms of hyaluronic acid (HA) per 32 cm² to about 28 micrograms of hyaluronic acid (HA) per 32 cm², about 12 micrograms of hyaluronic acid (HA) per 32 cm² to about 30 micrograms of hyaluronic acid (HA) per 32 cm², about 15 micrograms of hyaluronic acid (HA) per 32 cm² to about 20 micrograms of hyaluronic acid (HA) per 32 cm², about 15 micrograms of hyaluronic acid (HA) per 32 cm2 to about 25 micrograms of hyaluronic acid (HA) per 32 cm², about 15 micrograms of hyaluronic acid (HA) per 32 cm² to about 28 micrograms of hyaluronic acid (HA) per 32 cm², about 15 micrograms of hyaluronic acid (HA) per 32 cm² to about 30 micrograms of hyaluronic acid (HA) per 32 cm2, about 20 micrograms of hyaluronic acid (HA) per 32 cm² to about 25 micrograms of hyaluronic acid (HA) per 32 cm², about 20 micrograms of hyaluronic acid (HA) per 32 cm² to about 28 micrograms of hyaluronic acid (HA) per 32 cm2, about 20 micrograms of hyaluronic acid (HA) per 32 cm2 to about 30 micrograms of hyaluronic acid (HA) per 32 cm², about 25 micrograms of hyaluronic acid (HA) per 32 cm² to about 28 micrograms of hyaluronic acid (HA) per 32 cm², about 25 micrograms of hyaluronic acid (HA) per 32 cm² to about 30 micrograms of hyaluronic acid (HA) per 32 cm², or about 28 micrograms of hyaluronic acid (HA) per 32 cm² to about 30 micrograms of hyaluronic acid (HA) per 32 cm². In some embodiments, a fetal tissue product can comprise less than about 1 micrograms of hyaluronic acid (HA) per 32 cm², about 3 micrograms of hyaluronic acid (HA) per 32 cm², about 5 micrograms of hyaluronic acid (HA) per 32 cm², about 7 micrograms of hyaluronic acid (HA) per 32 cm2, about 9 micrograms of hyaluronic acid (HA) per 32 cm², about 10 micrograms of hyaluronic acid (HA) per 32 cm², about 12 micrograms of hyaluronic acid (HA) per 32 cm², about 15 micrograms of hyaluronic acid (HA) per 32 cm², about 20 micrograms of hyaluronic acid (HA) per 32 cm², about 25 micrograms of hyaluronic acid (HA) per 32 cm², about 28 micrograms of hyaluronic acid (HA) per 32 cm², or about 30 micrograms of hyaluronic acid (HA) per 32 cm². In some embodiments, a fetal tissue product can comprise less than at least about 1 micrograms of hyaluronic acid (HA) per 32 cm², about 3 micrograms of hyaluronic acid (HA) per 32 cm², about 5 micrograms of hyaluronic acid (HA) per 32 cm², about 7 micrograms of hyaluronic acid (HA) per 32 cm², about 9 micrograms of hyaluronic acid (HA) per 32 cm², about 10 micrograms of hyaluronic acid (HA) per 32 cm², about 12 micrograms of hyaluronic acid (HA) per 32 cm², about 15 micrograms of hyaluronic acid (HA) per 32 cm², about 20 micrograms of hyaluronic acid (HA) per 32 cm², about 25 micrograms of hyaluronic acid (HA) per 32 cm², or about 28 micrograms of hyaluronic acid (HA) per 32 cm². In some embodiments, a fetal tissue product can comprise about 3 micrograms of hyaluronic acid (HA) per 32 cm², about 5 micrograms of hyaluronic acid (HA) per 32 cm², about 7 micrograms of hyaluronic acid (HA) per 32 cm², about 9 micrograms of hyaluronic acid (HA) per 32 cm², about 10 micrograms of hyaluronic acid (HA) per 32 cm2, about 12 micrograms of hyaluronic acid (HA) per 32 cm2, about 15 micrograms of hyaluronic acid (HA) per 32 cm², about 20 micrograms of hyaluronic acid (HA) per 32 cm², about 25 micrograms of hyaluronic acid (HA) per 32 cm², about 28 micrograms of hyaluronic acid (HA) per 32 cm², or about 30 micrograms of hyaluronic acid (HA) per 32 cm².

In some embodiments, the HA of the AM product can be a low molecular weight HA, which is still covalently bonded to HC1 and then complexed to pentraxin 3. In some embodiments, a fetal tissue product or an amniotic membrane (AM) comprises reduced total HA content compared to an AM product that has not been terminally sterilized. In some embodiments, a fetal tissue product or an amniotic membrane (AM) comprising reduced total HA fetal tissue extract or product may be characterized by how increased lubrication or stickiness of the tissue product. In some embodiments, an amniotic membrane (AM) disclosed comprising reduced total HA content facilitates lubrication of the fetal support tissue product. In some embodiments, lubrication facilitates adherence of an AM tissue or tissue product to an ocular surface or ocular lens e.g., such as a corneal surface of an eye or on a surface of a contact lens (of any known contact lenses). In some embodiments, a fetal tissue product or an amniotic membrane (AM) product disclosed comprises reduced total HA content which may be characterized by increased adhesive property of an AM product to an ocular surface or ocular lens. In some embodiments an ocular surface comprises a native or natural lens of an eye, or an artificial lens. In some embodiments an ocular lens comprises a contact lens e.g., a collagen shield (CS), collagen corneal shield, or a bandage contact lens (BCL). In some embodiments, as disclosed herein an AM product comprises HC-HA/PTX3 complex comprising a potency, a total protein content as disclosed herein. In some embodiments, a potency of HC-HA/PTX3 complex may be preserved during terminal sterilization. In some embodiments, a potency of HC-HA/PTX3 complex comprises anti-inflammatory biological effects, anti-scarring activities, wound healing properties, or a combination thereof. In some embodiments, an AM product disclosed comprises high molecular weight pentraxin 3 (PTX3) in HC-HA/PTX3 complex which may be preserved during terminal sterilization.

In some embodiments, disclosed herein is a birth tissue or placental tissue comprising a fetal support tissue. In some embodiments, a fetal tissue comprises amniotic membrane, umbilical cord, umbilical cord amniotic membrane, chorion, amnion-chorion, amniotic stroma, amniotic jelly, Wharton's jelly, amniotic fluid, or a combination thereof.

In some embodiments, an AM tissue product comprises cryopreserved, hydrated amniotic membrane (AM).

In some embodiments, an AM tissue product comprises a circular-shaped AM tissue product. In some embodiments, a circular AM tissue product comprises an average diameter of about 2 mm to 13 mm, about 3 mm to 12 mm, about 4 mm to 11 mm, about 5 mm to 10 mm, or about 6 mm to 9 mm. In some embodiments, a circular-shaped AM tissue product comprises an average diameter of about 7 mm, 8 mm, 9 mm, 10 mm, 11 mm, or 12 mm. In some embodiments, a circular-shaped AM tissue product comprises an average diameter of about 8 mm. In some embodiments, a circular-shaped AM tissue product comprises an average diameter of about 10 mm. In some embodiments, a circular-shaped AM tissue product comprises an average diameter of about 12 mm. In some embodiments, a circular-shaped AM tissue product comprises tissue product that may be ringless.

In some embodiments, a circular-shaped AM tissue product comprises a thickness of about 5 mm to 12 mm, about 6 mm to 11 mm, about 7 mm to 10 mm, or about 8 mm to 9 mm.

In certain embodiments, the hydrated fetal tissue product can weigh about 50 milligrams, 100 milligrams, 100 milligrams, 200 milligrams, 300 milligrams, 400 milligrams, 500 milligrams, 600 milligrams, 700 milligrams, 800 milligrams, 900 milligrams, 1,000 milligrams, 1,500 milligrams, 2,000 milligrams or more. In certain embodiments, the fetal tissue product can weigh about 100 milligrams, 100 milligrams, 200 milligrams, 300 milligrams, 400 milligrams, 500 milligrams, 600 milligrams, 700 milligrams, 800 milligrams, 900 milligrams, 1,000 milligrams, 1,500 milligrams, 2,000 milligrams or less. In certain embodiments, the fetal tissue product can weigh about 50 milligrams, 100 milligrams, 100 milligrams, 200 milligrams, 300 milligrams, 400 milligrams, 500 milligrams, 600 milligrams, 700 milligrams, 800 milligrams, 900 milligrams, 1,000 milligrams, 1,500 milligrams, 2,000 milligrams.

### Shapes and Dimensions of Fetal Tissue Products

In some embodiments, the fetal tissue product is in any suitable shape (e.g., a square, a circle, a triangle, a rectangle, etcetera). In some embodiments, the fetal tissue is generated from a sheet of fetal tissue. In some embodiments, the sheet is flat.

In some embodiments, a fetal tissue product can comprise a fetal tissue that was previously frozen. In some embodiments, a fetal tissue product can comprise a sheet that is circular.

The fetal tissue products described herein can have any suitable dimensions. In some embodiments, a fetal tissue product can comprise a sheet having a diameter of less than about 25, 24, 23, 22, 21, 20, 19, 18, 17, or 16 millimeters. In some embodiments, a fetal tissue product can comprise a sheet having a diameter of greater than about 1, 2, 3, 4, 5, 6, 7, 8, 9, 10, 11 or 12 millimeters. In some embodiments, a fetal tissue product can comprise a sheet having a diameter of about 1, 2, 3, 4, 5, 6, 7, 8, 9, 10, 11, 12, 13, 14, 15, 16, 17, 18, 19, or 20 millimeters. In some embodiments, a fetal tissue product can comprise a sheet having a diameter from 8 millimeters to 16 millimeters. In some embodiments, a fetal tissue product can comprise a sheet having a diameter from about 1 millimeter to 16 millimeters. In some embodiments, a fetal tissue product can comprise a sheet having a diameter of about 1 millimeter. In some embodiments, a fetal tissue product can comprise a sheet having a diameter of about 3 millimeters. In some embodiments, a fetal tissue product can comprise a sheet having a diameter of about 4 millimeters. In some embodiments, a fetal tissue product can comprise a sheet having a diameter of about 5 millimeters. In some embodiments, a fetal tissue product can comprise a sheet having a diameter of about 6 millimeters. In some embodiments, a fetal tissue product can comprise a sheet having a diameter of about 7 millimeters. In some embodiments, a fetal tissue product can comprise a sheet having a diameter of about 8 millimeters. In some embodiments, a fetal tissue product can comprise a sheet having a diameter of about 9 millimeters. In some embodiments, a fetal tissue product can comprise a sheet having a diameter of about 10 millimeters. In some embodiments, a fetal tissue product can comprise a sheet having a diameter of about 11 millimeters. In some embodiments, a fetal tissue product can comprise a sheet having a diameter of about 12 or more millimeters.

The fetal tissue products described herein can have any suitable thickness compatible with a particular therapeutic indication. In some embodiments, a fetal tissue product can comprise a sheet having an average thickness of less than about 500 micrometers. In some embodiments, a fetal tissue product can comprise a sheet having an average thickness of less than about 200 micrometers. In some embodiments, a fetal tissue product can comprise a sheet having an average thickness of less than about 100 micrometers. In some embodiments, a fetal tissue product can comprise a sheet having an average thickness of less than about 50 micrometers. In some embodiments, a fetal tissue product can comprise a sheet having an average thickness of less than about 25 micrometers. In some embodiments, a fetal tissue product can comprise a sheet having an average thickness of greater than about 1 micrometer. In some embodiments, a fetal tissue product can comprise a sheet having an average thickness of greater than about 10 micrometers. In some embodiments, a fetal tissue product can comprise a sheet having an average thickness of greater than about 20 micrometers. In some embodiments, a fetal tissue product can comprise a sheet having an average thickness of greater than about 25 micrometers. In some embodiments, a fetal tissue product can comprise a sheet having an average thickness of greater than about 50 micrometers. In some embodiments, a fetal tissue product can comprise a sheet having an average thickness of greater than about 100 micrometers. In some embodiments, a fetal tissue product may comprise a sheet having an average thickness of greater than about 90 micrometers. In some embodiments, a fetal tissue product may comprise a sheet having an average thickness of greater than about 80 micrometers. In some embodiments, a fetal tissue product may comprise a sheet having an average thickness of greater than about 70 micrometers. In some embodiments, a fetal tissue product may comprise a sheet having an average thickness of greater than about 60 micrometers. In some embodiments, a fetal tissue product may comprise a sheet having an average thickness of between about 200 micrometers and about 1 micrometer. In some embodiments, a fetal tissue product may comprise a sheet having an average thickness of between about 100 micrometers and about 3 micrometers. In some embodiments, a fetal tissue product may comprise a sheet having an average thickness of between about 50 micrometers and about 5 micrometers. In some embodiments, a fetal tissue product may comprise a sheet having an average thickness of between about 30 micrometers and about 10 micrometers. In some embodiments, a fetal tissue product may comprise a sheet having an average thickness of between about 25 micrometers and about 15 micrometers. In some embodiments, a fetal tissue product may comprise a sheet having an average thickness of between about 12 micrometers and about 2. micrometers. In some embodiments, a fetal tissue product may comprise a sheet having an average thickness of between about 9 micrometers. In some embodiments, a fetal tissue product may comprise a sheet having an average thickness of between about 8 micrometers. In some embodiments, a fetal tissue product may comprise a sheet having an average thickness of between about 7 micrometers. In some embodiments, a fetal tissue product may comprise a sheet having an average thickness of between about 6 micrometers. In some embodiments, a fetal tissue product may comprise a sheet having an average thickness of greater than 50 micrometers. In some embodiments, a fetal tissue product may comprise a sheet having an average thickness of greater than 5 micrometers. In some embodiments, a fetal tissue product may comprise a sheet having an average thickness of greater than 7 micrometers. In some embodiments, a fetal tissue product may comprise a sheet having an average thickness of greater than 8 micrometers. In some embodiments, a fetal tissue product may comprise a sheet having an average thickness of greater than 10 micrometers. In some embodiments, a fetal tissue product may comprise a sheet having an average thickness of 9 micrometers. In some embodiments, a fetal tissue product may comprise a sheet having an average thickness of 8 micrometers. In some embodiments, a fetal tissue product may comprise a sheet having an average thickness of 7 micrometers. In some embodiments, a fetal tissue product may comprise a sheet having an average thickness 6 micrometers or less.

In some embodiments, a fetal tissue product may comprise a sheet that is substantially flat. In some embodiments, a fetal tissue product may comprise a fetal tissue that is not dehydrated. In some embodiments, a fetal tissue product may comprise water that is not removed from the fetal tissue. In some embodiments, a fetal tissue product may comprise cells where substantially all of the cells of a fetal tissue are dead.

### Packaging and Adhesiveness of Fetal Tissue Product (AM Product)

The AM product disclosed herein can be a sheet product that is not dehydrated (e.g., hydrated or not dry) and once the product is unpacked does not require rehydration. In some embodiments, a fetal tissue product or an AM product disclosed herein can be stored in 200 μl saline storage solution and the HC-HA/PTX3 activity of the product is preserved, while HA content is reduced. In some embodiments, placement of an AM product in saline e.g., during storage, may facilitate solubilization of HA from a product. In some embodiments, storage of an AM product in saline may facilitate adhesiveness of a tissue product. In some embodiments, a fetal tissue product or AM product disclosed comprises a total HA content of about 0.2 μg to about 20 μg of HA. In some embodiments, a fetal tissue product or AM product disclosed comprises a total HA content of about 0.0001, 0.001, 0.01, 0.02, 0.03, 0.04, 0.05, 0.06, 0.07, 0.08, 0.09, 0.1, 0.15 μg or more of HA. In some embodiments, a fetal tissue product or AM product disclosed comprises a total HA content of less than 2.0, 1.5, 1.0, 0.9, 0.8, 0.7, 0.6, 0.5, 0.4, 0.3, 0.2, 0.1 μg of HA content. In some embodiments, a fetal tissue product or AM product disclosed comprises a total HA content of 0.2 μg or less of HA. In some embodiments, a fetal tissue product or AM product disclosed comprises a total HA content of 0.4 μg or less of HA. In some embodiments, a fetal tissue product or AM product disclosed comprises a total HA content of 0.5 μg or less of HA. In some embodiments, a fetal tissue product or AM product disclosed comprises a total HA content of 0.6 μg or less of HA. In some embodiments, a fetal tissue product or AM product disclosed comprises a total HA content of 0.7 μg or less of HA. In some embodiments, storage of an AM product in saline may not facilitate any further solubilization of HA or adhesiveness.

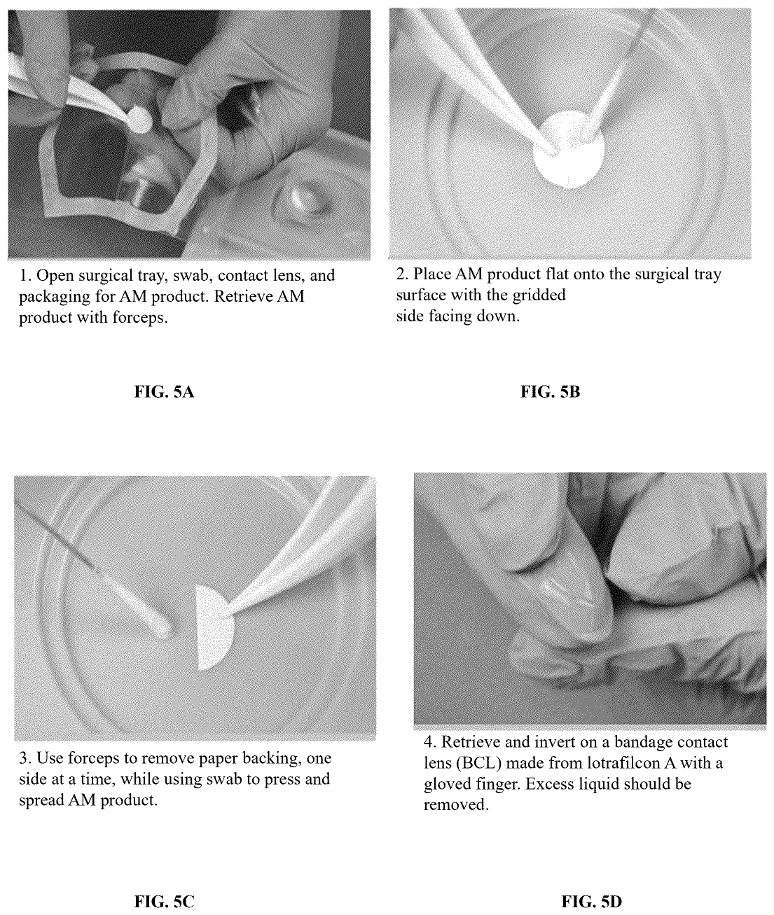

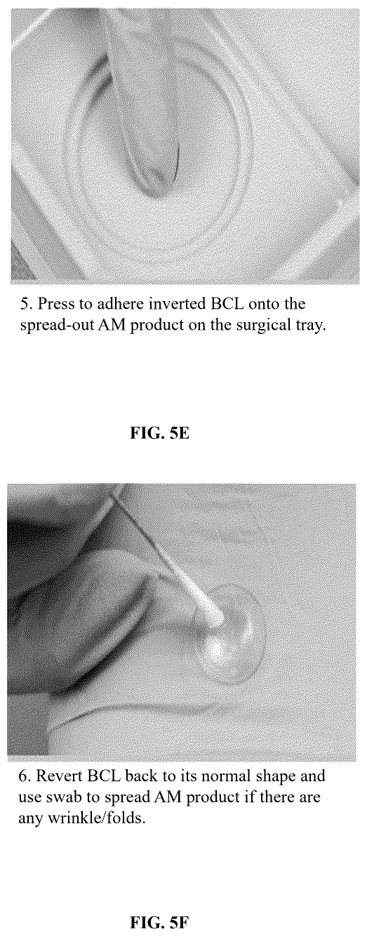

As indicated in FIG. 5A-FIG. 5F, the AM product is sealed in a pouch that has an inner and outer pouch. The AM product produced by the methods disclosed herein retains the water content so that it is not a dehydrated product but rather stays hydrated. The manufacturing and sterilization procedures disclosed produce an AM product having strong adhesive properties that enable it to adhere onto surfaces e.g., contact lens surfaces immediately without any prompting. The strong adhesive properties present on the AM product is seen on both the epithelial and stroma sides of the AM product. Biochemical Analysis data available related to the AM product of the present disclosure and the AmnioGraft shows both products retain HC-HA/PTX3 that is innate to fresh amniotic tissue. The AM product also has less hyaluronic acid (ug HA per cm2 AM tissue) compared to other amniotic products. Without being bound by theory this reduced HA content contributes to the strong adhesive properties of the AM product. Nonetheless, the AM product retains HA that is bound in the HC-HA/PTX3 complex. However, the HA amount of the AM product is less and of smaller molecular weight (see agarose gel electrophoresis data). The AM product is not a dehydrated product, it is hydrated, and terminally sterilized. Because it adheres on surfaces without need for suturing or gluing in place, the AM product can be placed directly on the surface of the eye, e.g., the corneal surface without any tools/vehicles and can be used to treat ocular conditions following adherence. Unpacking or following instructions in the shipment (FIG. 5A-5F). The product can be sealed in any suitable water tight and air-tight pouch to preserve sterility and bioactivity of the fetal tissue product.

Presented herein is a fetal tissue product or an AM product for use in a method of treating a disease or disorder of an eye comprising administering a fetal tissue product that can adhere to a surface of a contact lens to an eye of an individual. In some embodiments, a method of administering a fetal tissue product or an AM product to an eye can be done without adhering the fetal tissue product to a surface of a contact lens. In some embodiments, a method of administering a fetal tissue product to an eye can comprise adhering the fetal tissue product to the surface of a contact lens.

In some embodiments, a fetal tissue product (or AM product) can be processed by a sterilization manufacturing procedure thereby providing a product with surface adhesive properties. For example, a fetal tissue product provided herein comprises an adhesive property that allows application of the product on a surface of an eye without suturing, without any additional cross-linking agents, adhesives, or any ocular glue. In some embodiments, a fetal tissue product disclosed herein can be applied directly on an ocular surface of an eye. In some embodiments, a fetal tissue product disclosed herein can also be applied on a surface of a contact lens, e.g., a lotrafilcon A surface prior to placing the contact lens on an ocular surface of an eye.

In some embodiments, a fetal tissue product can adhere to a backing material. In some embodiments, a fetal tissue product can comprise a backing material that comprises a polyethersulfone (PES) polymer. In some embodiments, a fetal tissue product can comprise a backing material that is gridded. In some embodiments, a fetal tissue product may not comprise a ring-shaped support structure. In some embodiments, a fetal tissue product can be clear. In some embodiments, a fetal tissue product can be optically clear. In some embodiments, a fetal tissue product can be packaged in normal (0.9%) saline.

The fetal tissue products produced by the methods described herein can be shelf stable for an extended period of time increasing their utility and convenience for treating individuals. In some embodiments, a fetal tissue product can be stable at −20° C. to 25° C. for at least six months. In some embodiments, a fetal tissue product can be stable at 20° C. to 25° C. for at least nine months. In some embodiments, a fetal tissue product can be stable at 20° C. to 25° C. for at least one year. In some embodiments, a fetal tissue product can be stable at 20° C. to 25° C. for at least 18 months.

In some embodiments, a fetal tissue product can adhere to a surface e.g., a surface of a contact lens such as a lens comprising lotrafilcon A with a force of greater than 0.0001 pounds per square inch. In some embodiments, a fetal tissue product can adhere to a surface comprising lotrafilcon A with a force of greater than 0.001 pounds per square inch. In some embodiments, a fetal tissue product can adhere to a surface comprised of lotrafilcon A with a force of greater than 0.01 pounds per square inch. In some embodiments, a fetal tissue product can adhere to a surface comprising lotrafilcon A with a force of greater than 0.1 pounds per square inch. In some embodiments, a fetal tissue product can adhere to a surface comprising lotrafilcon A with a force of greater than 1 pound per square inch. In some embodiments, a fetal tissue product can adhere to a surface comprising lotrafilcon A with a force of greater than 2 pounds per square inch. In some embodiments, a fetal tissue product can adhere to a surface comprising lotrafilcon A with a force of greater than 5 pounds per square inch. In some embodiments, a fetal tissue product can adhere to a surface comprising lotrafilcon A with a force of greater than 10 pounds per square inch. In some embodiments, a fetal tissue product can adhere to a surface comprising lotrafilcon A with a force of greater than 15 pounds per square inch. In some embodiments, a fetal tissue product can adhere to a surface comprised of lotrafilcon A with a force of greater than 20 pounds per square inch. In some embodiments, a fetal tissue product can adhere to a surface comprising lotrafilcon A with a force of greater than about 0.0001 pounds per square inch to about 20 pounds per square inch. In some embodiments, a fetal tissue product can adhere to a surface comprising lotrafilcon A with a force of greater than about 0.0001 pounds per square inch to about 0.001 pounds per square inch, about 0.0001 pounds per square inch to about 0.01 pounds per square inch, about 0.0001 pounds per square inch to about 0.1 pounds per square inch, about 0.0001 pounds per square inch to about 0.5 pounds per square inch, about 0.0001 pounds per square inch to about 1 pounds per square inch, about 0.0001 pounds per square inch to about 1.5 pounds per square inch, about 0.0001 pounds per square inch to about 2 pounds per square inch, about 0.0001 pounds per square inch to about 5 pounds per square inch, about 0.0001 pounds per square inch to about 10 pounds per square inch, about 0.0001 pounds per square inch to about 15 pounds per square inch, about 0.0001 pounds per square inch to about 20 pounds per square inch, about 0.001 pounds per square inch to about 0.01 pounds per square inch, about 0.001 pounds per square inch to about 0.1 pounds per square inch, about 0.001 pounds per square inch to about 0.5 pounds per square inch, about 0.001 pounds per square inch to about 1 pounds per square inch, about 0.001 pounds per square inch to about 1.5 pounds per square inch, about 0.001 pounds per square inch to about 2 pounds per square inch, about 0.001 pounds per square inch to about 5 pounds per square inch, about 0.001 pounds per square inch to about 10 pounds per square inch, about 0.001 pounds per square inch to about 15 pounds per square inch, about 0.001 pounds per square inch to about 20 pounds per square inch, about 0.01 pounds per square inch to about 0.1 pounds per square inch, about 0.01 pounds per square inch to about 0.5 pounds per square inch, about 0.01 pounds per square inch to about 1 pounds per square inch, about 0.01 pounds per square inch to about 1.5 pounds per square inch, about 0.01 pounds per square inch to about 2 pounds per square inch, about 0.01 pounds per square inch to about 5 pounds per square inch, about 0.01 pounds per square inch to about 10 pounds per square inch, about 0.01 pounds per square inch to about 15 pounds per square inch, about 0.01 pounds per square inch to about 20 pounds per square inch, about 0.1 pounds per square inch to about 0.5 pounds per square inch, about 0.1 pounds per square inch to about 1 pounds per square inch, about 0.1 pounds per square inch to about 1.5 pounds per square inch, about 0.1 pounds per square inch to about 2 pounds per square inch, about 0.1 pounds per square inch to about 5 pounds per square inch, about 0.1 pounds per square inch to about 10 pounds per square inch, about 0.1 pounds per square inch to about 15 pounds per square inch, about 0.1 pounds per square inch to about 20 pounds per square inch, about 0.5 pounds per square inch to about 1 pounds per square inch, about 0.5 pounds per square inch to about 1.5 pounds per square inch, about 0.5 pounds per square inch to about 2 pounds per square inch, about 0.5 pounds per square inch to about 5 pounds per square inch, about 0.5 pounds per square inch to about 10 pounds per square inch, about 0.5 pounds per square inch to about 15 pounds per square inch, about 0.5 pounds per square inch to about 20 pounds per square inch, about 1 pounds per square inch to about 1.5 pounds per square inch, about 1 pounds per square inch to about 2 pounds per square inch, about 1 pounds per square inch to about 5 pounds per square inch, about 1 pounds per square inch to about 10 pounds per square inch, about 1 pounds per square inch to about 15 pounds per square inch, about 1 pounds per square inch to about 20 pounds per square inch, about 1.5 pounds per square inch to about 2 pounds per square inch, about 1.5 pounds per square inch to about 5 pounds per square inch, about 1.5 pounds per square inch to about 10 pounds per square inch, about 1.5 pounds per square inch to about 15 pounds per square inch, about 1.5 pounds per square inch to about 20 pounds per square inch, about 2 pounds per square inch to about 5 pounds per square inch, about 2 pounds per square inch to about 10 pounds per square inch, about 2 pounds per square inch to about 15 pounds per square inch, about 2 pounds per square inch to about 20 pounds per square inch, about 5 pounds per square inch to about 10 pounds per square inch, about 5 pounds per square inch to about 15 pounds per square inch, about 5 pounds per square inch to about 20 pounds per square inch, about 10 pounds per square inch to about 15 pounds per square inch, about 10 pounds per square inch to about 20 pounds per square inch, or about 15 pounds per square inch to about 20 pounds per square inch. In some embodiments, a fetal tissue product can adhere to a surface comprising lotrafilcon A with a force of greater than about 0.0001 pounds per square inch, about 0.001 pounds per square inch, about 0.01 pounds per square inch, about 0.1 pounds per square inch, about 0.5 pounds per square inch, about 1 pounds per square inch, about 1.5 pounds per square inch, about 2 pounds per square inch, about 5 pounds per square inch, about 10 pounds per square inch, about 15 pounds per square inch, or about 20 pounds per square inch. In some embodiments, a fetal tissue product can adhere to a surface comprising lotrafilcon A with a force of greater than at least about 0.0001 pounds per square inch, about 0.001 pounds per square inch, about 0.01 pounds per square inch, about 0.1 pounds per square inch, about 0.5 pounds per square inch, about 1 pounds per square inch, about 1.5 pounds per square inch, about 2 pounds per square inch, about 5 pounds per square inch, about 10 pounds per square inch, or about 15 pounds per square inch. In some embodiments, a fetal tissue product can adhere to a surface comprising lotrafilcon A with a force of greater than about 0.001 pounds per square inch, about 0.01 pounds per square inch, about 0.1 pounds per square inch, about 0.5 pounds per square inch, about 1 pounds per square inch, about 1.5 pounds per square inch, about 2 pounds per square inch, about 5 pounds per square inch, about 10 pounds per square inch, about 15 pounds per square inch, or about 20 pounds per square inch.

Presented herein is a fetal tissue product or an AM product that can exhibit increased adhesion to a surface comprising lotrafilcon A after sterilization with gamma irradiation at temperature of below Oo C. In some embodiments, a fetal tissue product can adhere to the contact lens such that when inserted into an eye of an individual the fetal tissue product contacts a surface of an eye of an individual.

In some embodiments, a fetal tissue product can comprise amniotic membrane (AM). In some embodiments, a fetal tissue product can comprise a placental amniotic membrane (PAM), an amniotic membrane (AM), an umbilical cord (UC), an umbilical cord amniotic membrane (UCAM), or any combination thereof. In some embodiments, a fetal tissue product can comprise a placental amniotic membrane (PAM). In some embodiments, a fetal tissue product can comprise an umbilical cord (UC). In some embodiments, a fetal tissue product can comprise an umbilical cord amniotic membrane (UCAM).

In some embodiments, a fetal tissue product can comprise placental amniotic membrane (AM). In some embodiments, a fetal tissue product does not comprise or is substantially free of umbilical cord, veins or arteries, Wharton's jelly or chorion.

### Biological Factors in Fetal Tissues

Disclosed herewith is a fetal tissue product or an AM product that contains bioactive factors e.g., HC-HA/PTX3 that can be preserved and utilized in various applications as disclosed herein. Fetal tissue disclosed herein, for example, amniotic membrane and umbilical cord contain several innate biological factors with numerous significant clinical efficacies disclosed herein. For example, HC-HA/PTX3 is preserved in the manufacturing or processing methods of the present application. The HC-HA/PTX3 preserved by the present disclosure contains of low weight (LMW) hyaluronan (HA) that is covalently linked with heavy chain (HC) 1 from inter-α-trypsin inhibitor and further complexed with pentraxin3 (PTX3). This HC-HA/PTX3 is one key active component of umbilical cord and amniotic membrane that is responsible for their wound healing effects. Accordingly, production of a fetal tissue product (e.g., an amniotic membrane and umbilical cord extract for use in wound healing) with a high yield of HC-HA/PTX3 is critical. Production of a fetal tissue product using a process that reduces or prevents the degradation of the HC-HA/PTX3 complex and other proteins of interest is while concentrating these biomolecules is important. In some embodiments, a fetal tissue product disclosed comprises HC-HA/PTX3 or HC-HA/PTX3 complex. In some embodiments, HC-HA/PTX3 disclosed herein comprises one or more biological activities or a potency of a fetal tissue product disclosed herein. Non-limiting examples of biological activities of the present fetal tissue or amniotic membrane tissue product comprising HC-HA/PTX3 are illustrated in FIG. 1.

### Dosage Forms

Provided below are dosage forms of the compositions described herein comprising composition comprising a fetal tissue product or AM product. The product form can comprise a composition comprising an AM graft, a sheet, a strip, a device, or any combination thereof. The methods or steps of placing the present disclosure onto an eye of the subject are disclosed herein. The AM product can be administered to an individual being treated e.g., a human subject, as determined by the prescribing physician.

Presented herein is a fetal tissue product (or AM product, used interchangeably throughout) processed by a sterilization manufacturing procedure which allows the product to comprise adhesiveness. For example, an AM product provided herein comprises an adhesive property that allows application of the product on a surface of an eye without suturing, without any additional tissue adhesives, or any ocular glue. In some embodiments, an AM product provided herein can be placed directly onto a surface of an eye of a subject. In some embodiments, an AM product provided herein can first be placed directly onto an external device e.g., a contact lens of any type disclosed herein or contact lens for use in an eye surface. In some embodiments, an AM product provided herein can be placed on a surface of an eye or ocular surface, and it can adhere for at least 1 hour, 2 hours, 3 hours, 6 hours, 12 hours, 24 hours, 96 hours or more. In some embodiments, an AM product provided herein can be placed on a surface of an eye or ocular surface, and it can adhere for at least day, at least 2 days, at least 3, 4, 5, 6, 7, 8, 9, 10, 11, 12, 13, 14, 15 or more days. In some embodiments, an AM product disclosed herein can be placed on a surface of an eye or ocular surface, and it can adhere for about 1 day to about 15 days. In some embodiments, an AM product disclosed herein can be placed on a surface of an eye or an ocular surface, and it can adhere for about 1 day to about 15 days. In some embodiments, an AM product disclosed herein can be placed on a surface of an eye or an ocular surface, and it can adhere for about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 15 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 15 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 15 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 15 days, about 5 days to about 6 days, about 5 days to about 7 days, about 5 days to about 8 days, about 5 days to about 9 days, about 5 days to about 10 days, about 5 days to about 11 days, about 5 days to about 15 days, about 6 days to about 7 days, about 6 days to about 8 days, about 6 days to about 9 days, about 6 days to about 10 days, about 6 days to about 11 days, about 6 days to about 15 days, about 7 days to about 8 days, about 7 days to about 9 days, about 7 days to about 10 days, about 7 days to about 11 days, about 7 days to about 15 days, about 8 days to about 9 days, about 8 days to about 10 days, about 8 days to about 11 days, about 8 days to about 15 days, about 9 days to about 10 days, about 9 days to about 11 days, about 9 days to about 15 days, about 10 days to about 11 days, about 10 days to about 15 days, or about 11 days to about 15 days. In some embodiments, an AM product disclosed herein can be placed on a surface of an eye or an ocular surface, and it can adhere for about 1 day, about 2 days, about 3 days, about 4 days, about 5 days, about 6 days, about 7 days, about 8 days, about 9 days, about 10 days, about 11 days, or about 15 days. In some embodiments, an AM product disclosed herein can be placed on a surface of an eye or an ocular surface, and it can adhere for at least about 1 day, about 2 days, about 3 days, about 4 days, about 5 days, about 6 days, about 7 days, about 8 days, about 9 days, about 10 days, or about 11 days. In some embodiments, an AM product disclosed herein can be placed on a surface of an eye or an ocular surface, and it can adhere for about 2 days, about 3 days, about 4 days, about 5 days, about 6 days, about 7 days, about 8 days, about 9 days, about 10 days, about 11 days, or about 15 days. In some embodiments, an AM product disclosed herein can be placed on a surface of an eye or an ocular surface, and it can adhere for more than 15 days.

### Fetal Tissues

In some embodiments, a fetal tissue comprises a fetal tissue graft, a fetal tissue sheet, a fetal tissue extract, or any combinations thereof. In some embodiments, the fetal tissue of the present invention disclosure is wet, cryopreserved, and terminally sterilized but still preserves native HC-HA/PTX3. In some embodiments, the fetal tissue is an extract of a fetal tissue. In some embodiments, the fetal tissue is a placental amniotic membrane, amniotic membrane, placenta, amniotic stroma, amniotic jelly, or any combination thereof. In certain embodiments,

### Generation of Fetal Tissues

In some embodiments, the fetal tissue is derived from an umbilical cord (UC) tissue. In some embodiments, the fetal tissue is derived from an amniotic membrane (AM) tissue. In some embodiments, the fetal tissue is derived from an umbilical cord amniotic membrane tissue. In some embodiments, the fetal tissue comprises isolated fetal tissue that does not comprise a vein or an artery. In some embodiments, the fetal tissue comprises: isolated fetal tissue that does not comprise a vein or an artery, a cell with metabolic activity, active HIV-1, active HIV-2, active HTLV-1, active hepatitis B, active hepatitis C, active West Nile Virus, active cytomegalovirus, active human transmissible spongiform encephalopathy, or active Treponema pallidum, wherein the natural structural integrity of the fetal tissue is substantially preserved for at least 15 days after initial procurement. In some embodiments, the biological activity of HC-HA/PTX3 in the fetal tissue is substantially preserved. In some embodiments, the biological activity of HC-HA/PTX3 complex in the fetal tissue is substantially preserved for at least 15 days. In some embodiments, the biological and structural integrity of the fetal tissue is substantially preserved for at least 20 days after initial procurement. In some embodiments, the biological and structural integrity of the fetal tissue is substantially preserved for at least 25 days after initial procurement. In some embodiments, the biological and structural integrity of the fetal tissue is substantially preserved for at least 30 days after initial procurement. In some embodiments, the biological and structural integrity of the fetal tissue is substantially preserved for at least 35 days after initial procurement. In some embodiments, the biological and structural integrity of the fetal tissue is substantially preserved for at least 40 days after initial procurement. In some embodiments, the biological and structural integrity of the fetal tissue is substantially preserved for at least 45 days after initial procurement. In some embodiments, the biological and structural integrity of the fetal tissue is substantially preserved for at least 50 days after initial procurement. In some embodiments, the biological and structural integrity of the fetal tissue is substantially preserved for at least 55 days after initial procurement. In some embodiments, the biological and structural integrity of the fetal tissue is substantially preserved for at least 60 days after initial procurement. In some embodiments, the biological and structural integrity of the fetal tissue is substantially preserved for at least 90 days after initial procurement. In some embodiments, the biological and structural integrity of the fetal tissue is substantially preserved for at least 180 days after initial procurement. In some embodiments, the biological and structural integrity of the fetal tissue is substantially preserved for at least 1 year after initial procurement. In some embodiments, the biological and structural integrity of the fetal tissue is substantially preserved for at least 2 years after initial procurement. In some embodiments, the biological and structural integrity of the fetal tissue is substantially preserved for at least 3 years after initial procurement. In some embodiments, the biological and structural integrity of the fetal tissue is substantially preserved for at least 4 years after initial procurement. In some embodiments, the biological and structural integrity of the fetal tissue is substantially preserved for at least 5 years after initial procurement. In some embodiments, the fetal tissue is obtained from a human, a non-human primate, a cow, or a pig.

In some embodiments, the fetal tissue is kept below 0° C. until donor and specimen eligibility has been determined. In some embodiments, the fetal tissue is kept from between 0° C. to −80° C. until donor and specimen eligibility has been determined. In some embodiments, storing the fetal tissue at −80° C. kills substantially all cells found in the fetal tissue. In some embodiments, storing the fetal tissue at −80° C. kills substantially all cells found in the fetal tissue while maintaining or increasing the biological activity of the fetal tissue (e.g., its anti-inflammatory, anti-scarring, anti-antigenic, and anti-adhesion properties) relative to fresh (i.e., non-frozen) fetal tissue. In some embodiments, storing the fetal tissue at −80° C. results in the loss of metabolic activity in substantially all cells found in the fetal tissue. In some embodiments, the fetal tissue can be hydrated and not dry or dried. In some embodiments, the fetal tissue is not dehydrated. In some embodiments, a fetal tissue product or an AM product of the present disclosure does not need to be rehydrated. In some embodiments, a fetal tissue product or an AM product disclosed herein is not rehydrated. In some embodiments, a fetal tissue product or AM product disclosed herein is not dry. In some embodiments, a fetal tissue product or AM product disclosed herein is not dried but retains water during the manufacturing or processing. In some embodiments, a fetal tissue product or AM product disclosed herein is not dehydrated.

### Processing of Fetal Tissue

In some embodiments, processing is done following Good Tissue Practices (GTP) to ensure that no contaminants are introduced into the fetal tissue.

In some embodiments, placental amniotic membrane tissue is harvested from any suitable donor source (e.g., a hospital or tissue bank) to prepare for the placental amniotic membrane sheet. In some embodiments, placental amniotic membrane tissue is obtained from any mammal, such as a human, non-human primate, cow, or pig. All processing is done following Good Tissue Practices (GTP) to ensure that no contaminants are introduced into the placental amniotic membrane sheet. In some embodiments, the harvested placental amniotic membrane is evaluated for HIV-1, HIV-2, HTLV-1, hepatitis B and C, West Nile virus, cytomegalovirus, human transmissible spongiform encephalopathy (e.g., Creutzfeldt-Jakob disease) and/or Treponema pallidum using FDA licensed screening test. Any indication that the tissue is contaminated with HIV-1, HIV-2, HTLV-1, hepatitis B and C, West Nile virus, or cytomegalovirus can result in the immediate quarantine and subsequent destruct of the tissue specimen. In some embodiments, the donor's medical records are examined for risk factors for and clinical evidence of hepatitis B, hepatitis C, or HIV infection. Any indication that the donor has risk factors for, and/or clinical evidence of, infection with HIV-1, HIV-2, HTLV-1, hepatitis B and C, West Nile virus, cytomegalovirus, human transmissible spongiform encephalopathy (e.g., Creutzfeldt-Jakob disease) and/or Treponema pallidum can result in the immediate quarantine and subsequent destruct of the tissue specimen. In some embodiments, the placental amniotic membrane is frozen. In some embodiments, the placental amniotic membrane is not frozen. If the placental amniotic membrane is not frozen, it can be processed as described below immediately.

In some embodiments, substantially all of the blood is removed from the placental amniotic membrane. In some embodiments, substantially all of the blood is removed from the placental amniotic membrane before the placental amniotic membrane is frozen. In some embodiments, substantially all of the blood is removed from the arteries and veins of the placental amniotic membrane. In some embodiments, blood is not removed from the amniotic membrane (AM). In some embodiments, blood is not removed from the placental amniotic membrane before the placental amniotic membrane is frozen. In some embodiments, the placental amniotic membrane tissue is washed with buffer with agitation to remove excess blood and tissue. In some embodiments, washing with agitation reduces the wash time. In some embodiments, the placental amniotic membrane tissue is contacted with a buffer to remove substantially all of the red blood cells. In some embodiments, the placental amniotic membrane tissue is lyophilized, cryopreserved, and/or terminally sterilized.

In some embodiments, the placental amniotic membrane is washed with a hypertonic or isotonic buffer or tissue culture media. In some embodiments, the placental amniotic membrane is washed with a hypotonic buffer or tissue culture media. In some embodiments, the placental amniotic membrane is washed with an isotonic buffer or tissue culture media. In some embodiments, the placental amniotic membrane is washed with saline. In some embodiments, the placental amniotic membrane is washed with PBS. In some embodiments, the placental amniotic membrane is washed with PBS 1×. In some embodiments, the placental amniotic membrane is washed with a TRIS-buffered saline. In some embodiments, the placental amniotic membrane is washed with a HEPES-buffered saline. In some embodiments, the placental amniotic membrane is washed with Ringer's solution. In some embodiments, the placental amniotic membrane is washed with Hartmann's solution. In some embodiments, the placental amniotic membrane is washed with EBSS. In some embodiments, the placental amniotic membrane is washed with HBSS. In some embodiments, the placental amniotic membrane is washed with Tyrode's Salt Solution. In some embodiments, the placental amniotic membrane is washed with Gey's Balanced Salt Solution. In some embodiments, the placental amniotic membrane is washed with Eagle's minimal essential medium (DMEM). In some embodiments, the placental amniotic membrane is washed with 0.9% saline. In some embodiments, the placental amniotic membrane is washed with Eagle's minimum essential medium (EMEM). In some embodiments, the placental amniotic membrane is washed with Glasgow minimum essential medium (GMEM). In some embodiments, the placental amniotic membrane is washed with Roswell Park Memorial Institute (RPMI) 1640 medium.

In some embodiments, a section of the placental amniotic membrane is then cut longitudinally (e.g., using a scalpel or scissors). In some embodiments, the section of the placental amniotic membrane is not cut into halves. In some embodiments, the section of the placental amniotic membrane is cut into two halves.

In some embodiments, the cut placental amniotic membrane tissue is optionally washed again with buffer to further remove excess blood and tissue. In some embodiments, the placental amniotic membrane is fastened onto a substrate (e.g., a Styrofoam board) using any suitable method (e.g., it is fastened with needles or pins (e.g., T pins)). In some embodiments, the placental amniotic membrane is stabilized with a substrate (e.g., absorbent towel cloth, drape).

In some embodiments, substantially all of the blood is removed from the fetal tissue (e.g., from any arteries and veins found in the fetal tissue, and blood that has infiltrated into the tissue). In some embodiments, substantially all of the blood is removed prior to or after it is frozen. In some embodiments, the blood is substantially removed prior to freezing. In some embodiments, the blood is substantially removed after freezing the tissue.

In some embodiments, the fetal tissue is washed with buffer with agitation to remove excess blood and tissue prior to freezing the tissue. In some embodiments, the fetal tissue is washed with buffer with agitation to remove excess blood and tissue after thawing frozen tissue. In some embodiments, the fetal tissue is a fetal tissue graft or sheet. In some embodiments, isolated fetal tissue is used to generate a fetal tissue graft or sheet prior freezing. In some embodiments, the fetal tissue is cut into multiple sections (e.g., using a scalpel). The size of the sections depends on the desired use of the fetal tissue graft or sheet derived from the fetal tissue. In some embodiments, the cut fetal tissue is optionally washed again with buffer to further remove excess blood and tissue.

In some embodiments, the fetal tissue is contacted with buffer under agitation to remove substantially all remaining red blood cells. In some embodiments, the fetal tissue is contacted with a buffer for 10 minutes, 15 minutes, 20 minutes, 25 minutes, 30 minutes, 40 minutes, 1 hour, 2 hours, 3 hours, 4 hours, 5 hours, 6 hours, 12 hours, 18 hours, 24 hours, or more than 24 hours. In some embodiments, the placental amniotic membrane is contacted with a buffer for 2 days, 3 days, 4 days, 5 days, 6 days, 7 days, 1 week, 2 weeks, 3 weeks, 4 weeks, or more than 4 weeks.

In some embodiments, a placental amniotic membrane, or an amniotic membrane (AM) is cut and prepared to form AM sheet. In some embodiments, the AM is cut into multiple sections (e.g., using a scalpel). In some embodiments, a section of the AM is then cut longitudinally (e.g., using a scalpel or scissors). In some embodiments, the section of the AM is not cut into halves. In some embodiments, the section of the AM is cut into two halves. In some embodiments, the AM sheet is cut into strips or threads.

The AM can be cut while practicing aseptic technique under a laminar flow hood. In some embodiments, the AM is cut into desired shapes and sizes with a scalpel using a ruler as a guide. In some embodiments, the AM is cut to about 1.5 cm by about 0.3 cm using a scalpel and ruler with grids. In some embodiments, the AM is cut to about 0.5 cm×about 0.1 cm, 0.5 cm×about 0.25 cm, about 0.5 cm×about 0.5 cm, about 0.5 cm×about 0.75 cm, about 0.5 cm×about 1 cm, about 0.5 cm×about 2 cm, about 0.5 cm×about 3 cm, about 0.5 cm×about 4 cm, about 0.5 cm×about 5 cm, or greater. In some embodiments, the AM sheet is about 1 cm×about 0.1 cm, 1 cm×about 0.25 cm, about 1 cm×about 0.5 cm, about 1 cm×about 0.75 cm, about 1 cm×about 1 cm, about 1 cm×about 2 cm, about 1 cm×about 3 cm, about 1 cm×about 4 cm, about 1 cm×about 5 cm, or greater. In some embodiments, the AM sheet is about 1.5 cm×about 0.1 cm, 1.5 cm×about 0.25 cm, about 1.5 cm×about 0.5 cm, about 1.5 cm×about 0.75 cm, about 1.5 cm×about 1 cm, about 1.5 cm×about 2 cm, about 1.5 cm×about 3 cm, about 1.5 cm×about 4 cm, about 1.5 cm×about 5 cm, or greater using a scalpel and ruler with grids. In some embodiments, the cut AM is stored in −20° C. In some embodiments, the AM is further subject to terminal sterilization by any suitable (e.g., medically acceptable) method including, but not limited to, gamma radiation, electron beam radiation, X-ray radiation, and UV radiation. In some embodiments, radiation is used with dry ice. In some embodiments, radiation is used without dry ice. In some embodiments, the AM is exposed to radiation with dry ice for a first period of time and exposed to radiation without dry ice for a second period of time. In some embodiments, the AM is exposed to gamma radiation for a period of time sufficient to sterilize the AM. In some embodiments, the AM is exposed to electron beam (E-Beam) sterilization for a period of time sufficient to sterilize the AM.

In some embodiments, the AM is exposed to electron beam (E-Beam) sterilization for a period of time sufficient to sterilize the AM. In some embodiments, the AM is further sterilized by gamma-irradiation at about 25 to about 43 kGy. In some embodiments, the AM is further sterilized by gamma-irradiation at about 10 to about 75 kGy. In some embodiments, the AM is further sterilized by gamma-irradiation at about 10, 15, 20, 25, 30, 35, 40, 45, 50, 55, 60, 65, 70, or more than 70 kGy. In some embodiments, the AM is exposed to electron beam (E-Beam) sterilization for a period of time sufficient to sterilize the AM. In some embodiments, the AM is further sterilized by E-Beam radiation at about 25 to about 43 kGy. In some embodiments, the AM is further sterilized by E-Beam radiation at about 10 to about 75 kGy. In some embodiments, the AM is further sterilized by E-Beam radiation at about 10, 15, 20, 25, 30, 35, 40, 45, 50, 55, 60, 65, 70, or more than 70 kGy. In some embodiments, the cut AM tissue is optionally washed again with buffer to further remove excess blood and tissue.

In some embodiments, the AM sheet comprises cells that are all, substantially all, or mostly dead. In some embodiments, the AM sheet is devoid of cells with metabolic activity.

In some embodiments, the AM sheet is hydrated. In some embodiments, the AM sheet is obtained from frozen or previously frozen AM. In some embodiments, the AM sheet is substantially flattened. In some embodiments, the AM sheet is semi-circular, circular, rectangular, or tubular. In some embodiments, the AM sheet is formed as a thread. In some embodiments, the AM sheet is configured to dissolve over time. In some embodiments, the sheet dissolves over a period of about 2 days, about 3 days, about 4 days, about 5 days, about 6 days, about 7 days, about 10 days, about 2 weeks, about 3 weeks, about 1 month, or about 2 months. In some embodiments, the AM sheet is about 0.5 cm×about 0.1 cm, 0.5 cm×about 0.25 cm, about 0.5 cm×about 0.5 cm, about 0.5 cm×about 0.75 cm, about 0.5 cm×about 1 cm, about 0.5 cm×about 2 cm, about 0.5 cm×about 3 cm, about 0.5 cm×about 4 cm, about 0.5 cm×about 5 cm, or greater. In some embodiments, the AM sheet is about 1 cm×about 0.1 cm, 1 cm×about 0.25 cm, about 1 cm×about 0.5 cm, about 1 cm×about 0.75 cm, about 1 cm×about 1 cm, about 1 cm×about 2 cm, about 1 cm×about 3 cm, about 1 cm×about 4 cm, about 1 cm×about 5 cm, or greater. In some embodiments, the AM sheet is about 1.5 cm×about 0.1 cm, 1.5 cm×about 0.25 cm, about 1.5 cm×about 0.5 cm, about 1.5 cm×about 0.75 cm, about 1.5 cm×about 1 cm, about 1.5 cm×about 2 cm, about 1.5 cm×about 3 cm, about 1.5 cm×about 4 cm, about 1.5 cm×about 5 cm, or greater. In some embodiments, the AM sheet is about 2 cm×about 0.1 cm, 2 cm×about 0.25 cm, about 2 cm×about 0.5 cm, about 2 cm×about 0.75 cm, about 2 cm×about 1 cm, about 2 cm×about 2 cm, about 2 cm×about 3 cm, about 2 cm×about 4 cm, about 2 cm×about 5 cm, or greater. In some embodiments, the AM sheet is about 2.5 cm×about 0.1 cm, 2.5 cm×about 0.25 cm, about 2.5 cm×about 0.5 cm, about 2.5 cm×about 0.75 cm, about 2.5 cm×about 1 cm, about 2.5 cm×about 2 cm, about 2.5 cm×about 3 cm, about 2.5 cm×about 4 cm, about 2.5 cm×about 5 cm, or greater. In some embodiments, the AM sheet is about 3 cm×about 0.1 cm, 3 cm×about 0.25 cm, about 3 cm×about 0.5 cm, about 3 cm×about 0.75 cm, about 3 cm×about 1 cm, about 3 cm×about 2 cm, about 3 cm×about 3 cm, about 3 cm×about 4 cm, about 3 cm×about 5 cm, or greater. In some embodiments, the AM sheet is about 1.0-2.0 cm by about 0.1-0.5 cm. In some embodiments, the AM sheet is about 1.5 cm by about 0.3 cm. In certain embodiments the AM sheet is rectangular with one side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters and the other side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters. In certain embodiments the AM sheet is rectangular with one side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters or less and the other side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters or less. In certain embodiments the AM sheet is rectangular with one side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters or greater and the other side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters or greater. In certain embodiments the AM sheet is square with a side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters. In certain embodiments the AM sheet is square with a side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters or greater. In certain embodiments the AM sheet is square with a side having a length of about 0.5, 1.0. 1.5, 2.0, 2.5, 3.0, 3.5, 4.0, 4.5, 5.0, 5.5, 6.0, 6.5, 7.0, 7.5, 8.0, 8.5 9.0, 9.5, or 10 centimeters or more.

In some embodiments, the AM sheet is cut to a prescribed size using a scalpel. In some embodiments, the scalpel is disposable. In some embodiments, the scalpel is reusable. In some embodiments, the scalpel is combined with a ruler to obtain a straight cut of the AM. In some embodiments, the AM sheet is placed over a backing paper (e.g., PES backing paper), thereby reducing the tendency for the AM to slide while being cut. In some embodiments, the AM sheet is cut with scissors. In some embodiments, the scissors are reusable. In some embodiments, the scissors are disposable.

In some embodiments, a fetal tissue product can comprise a fetal tissue that is terminally sterilized. In some embodiments, a fetal tissue product can comprise a fetal tissue that is terminally sterilized by gamma irradiation or electron beam sterilization. In some embodiments, a fetal tissue product can comprise substantially all of the cells of a fetal tissue that are dead. In some embodiments, a fetal tissue product can be terminally sterilized at a temperature at or below 0° C. In some embodiments, a fetal tissue product (or AM product, used interchangeably throughout) can be processed by a sterilization manufacturing procedure thereby providing a product with surface adhesive properties. For example, a fetal tissue product provided herein comprises an adhesive property that allows application of the product on a surface of an eye without suturing, without any additional tissue adhesives, or any ocular glue. In some embodiments, a fetal tissue product disclosed herein can be applied directly on an ocular surface of an eye. In some embodiments, a fetal tissue product disclosed herein can also be applied on a surface of a contact lens, e.g., a lotrafilcon A surface prior to placing the contact lens on an ocular surface of an eye.

### Cryopreservation

In some embodiments, the fetal tissue is then frozen for cryopreservation. The process of cryopreserving the fetal tissue does not destroy the integrity of a fetal tissue extracellular matrix. In some embodiments, the fetal tissue is exposed to a liquid gas (e.g., liquid nitrogen or liquid hydrogen). In some embodiments, the fetal tissue is exposed to liquid nitrogen. In some embodiments, the fetal tissue does not contact the liquid gas. In some embodiments, the fetal tissue is placed in a container and the container is contacted with liquid gas. In some embodiments, the fetal tissue is exposed to the liquid gas until the fetal tissue is frozen before or after cutting the tissue.

In some embodiments, the fetal tissue is cut before sterilization. In some embodiments, the fetal tissue is cut after sterilization. In some embodiments, the fetal tissue is cryopreserved before or after being cut into pieces. tissue or cryopreserved or after sterilization. In some embodiments, a cryopreservation method for processing a fetal tissue product disclosed herein is conducted under ice or cold conditions. In some embodiments, a cryopreservation method of the present disclosure utilizes a cryopreservation and/or a terminal sterilization method as described in U.S. Pat. Nos. 9,682,160 and 9,931,423 incorporated by reference herein in their entirety.

### Sterilization

In some embodiments, the fetal tissue is subject to sterilization by any suitable method. Fetal tissues, in some embodiments, are sterilized by gamma irradiation or electron beam irradiation which makes the fetal tissue free of contamination by living microorganisms.

In some embodiments, a fetal tissue product or AM product comprising HC-HA/PTX3 disclosed herein is subject to terminal sterilization by any suitable (e.g., medically acceptable) method. In some embodiments, a fetal tissue product disclosed herein is exposed to gamma radiation for a period of time sufficient to sterilize the fetal tissue product disclosed herein.

In some embodiments, a fetal tissue product or AM product comprising HC-HA/PTX3 disclosed herein is exposed to gamma radiation at about 10 to about 75 kilogray (kGy) for a period of time sufficient to sterilize the fetal tissue product. In some embodiments, a fetal tissue product disclosed herein is exposed to gamma radiation at about 10 to about 30 kGy for a period of time sufficient to sterilize the fetal tissue. In some embodiments, a fetal tissue product disclosed herein is exposed to gamma radiation at about 15 to about 30 kGy for a period of time sufficient to sterilize the fetal tissue. In some embodiments, a fetal tissue product disclosed herein is exposed to gamma radiation at about 25 kGy for a period of time sufficient to sterilize the fetal tissue. In some embodiments, a fetal tissue product disclosed herein is exposed to gamma radiation at about 17.5 kGy for a period of time sufficient to sterilize the fetal tissue product.

In some embodiments the fetal tissue product disclosed herein is subject to electron beam (E-Beam) sterilization. In some embodiments, a fetal tissue disclosed herein is exposed to E-Beam radiation at about 10 to about 75 kilogray for a period of time sufficient to sterilize the fetal tissue. In some embodiments, a fetal tissue disclosed herein is exposed to E-Beam radiation at about 10 to about 30 kGy for a period of time sufficient to sterilize the fetal tissue. In some embodiments, a fetal tissue disclosed herein is exposed to E-Beam radiation at about 15 to about 30 kGy for a period of time sufficient to sterilize the fetal tissue. In some embodiments, a fetal tissue disclosed herein is exposed to E-Beam radiation at about 25 kGy for a period of time sufficient to sterilize the fetal tissue. In some embodiments, a fetal tissue disclosed herein is exposed to E-Beam radiation at about 17.5 kGy for a period of time sufficient to sterilize the fetal tissue.

In some embodiments, a fetal tissue product disclosed herein is exposed to an electron beam for a period of time sufficient to sterilize the fetal tissue product. In some embodiments, a fetal tissue product disclosed herein is exposed to X-ray radiation for a period of time sufficient to sterilize the fetal tissue product. In some embodiments, a fetal tissue product disclosed herein is exposed to UV radiation for a period of time sufficient to sterilize the fetal tissue product.

Provided herein, in certain embodiments, are methods for preparing a fetal tissue, wherein a method results in an improved percentage of hyaluronic acid (HA) recovered. In some embodiments, at least or about 5%, 10%, 20%, 30%, 40%, 50%, 60%, 65%, 75% 80%, 85%, 90%, 95%, 99%, or more than 99% of the HA is recovered. In some embodiments, the HA is low molecular weight (LMW HA). In some embodiments, the methods result in an improved percentage of HC-HA/PTX3. In some embodiments, at least or about 30%, 40%, 50%, 60%, 65%, 75% 80%, 85%, 90%, 95%, 99%, or more than 99% of the HC-HA/PTX3 is recovered.

Methods as described herein, in certain embodiments, result in removal of particulates or degradants. In some embodiments, the methods described herein result in at least or about 75% 80%, 85%, 90%, 95%, 99%, or more than 99% of the particulates or degradants removed. In some embodiments, the particulates or degradants comprise chloride. In some embodiments, a fetal tissue product or AM product disclosed can be attached to the substrate. In some embodiments, a substrate can be nitrocellulose paper (NC). In some embodiments, a substrate can be nylon membrane (NM). In some embodiments, a substrate can polyethersulfone membrane (PES).

### Ophthalmic Device

Disclosed herein in some embodiments is an ophthalmic device comprising a fetal tissue product or AM product adhered to or contacting a substrate. In some embodiments, an ophthalmic device comprises the AM tissue product disclosed herein that is cryopreserved or terminally sterilized adhered to or placed on a substrate compatible with being placed in the eye of an individual. In some embodiments, an ophthalmic device disclosed comprises a substrate. In some embodiments, an ophthalmic device disclosed comprises a substrate comprising an artificial contact lens. In some embodiments, an ophthalmic device disclosed comprises a substrate comprising a collagen shield. In some embodiments, an artificial contact lens comprises any known artificial contact lens that may be used on a surface of an eye. In some embodiments, an eye may comprise an eye of a mammal, an animal, a human animal or a non-human animal, or a bird. In some embodiments, an artificial contact lens comprises a lens for use on a human eye. Non-limiting examples of contact lenses that may comprise a substrate of an ophthalmic device disclosure comprises a soft lens comprising for example, Senofilcon A, balafilcon A, methafilcon A, nelfilcon A hydrogel or silicon hydrogel. In some embodiments, an ophthalmic device may not be cross-linked to the substrate. Non-limiting examples of soft contact lenses may include: Acuvue Oasys (ACUVUE® OASYS), balafilcon A (PureVision®), lotrafilcon B (Air Optix® Night & Day Aqua®), Dailies® AquaComfort Plus®, Biofinity® (comfilcon A material). In some embodiments, a contact lens may be a rigid-gas permeable (RGP) lens. In some embodiments, a substrate comprises a collagen shield or a collagen corneal shield. In some embodiments, a substrate comprises a bandage contact lens. In some embodiments, an ophthalmic device comprises a substrate comprising a circular structure, square structure, rectangular structure or an irregular structure. In some embodiments, a substrate may be circular-shaped. In some embodiments, a substrate may be configured to be placed on a surface of an eye. In some embodiments, a fetal tissue product disclosed herein may be delivered to the surface of an eye. In some embodiments, delivery is via a contact lens. In some embodiments, a substrate comprising an adhered fetal tissue product may be placed on a surface of an eye. In some embodiments, a substrate comprising a fetal tissue product disclosed may be delivered to an eye using methods as used in delivering or placing a contact lens to the surface of an eye e.g., a corneal surface. Anon-limiting example comprises using an index finger to deliver a contact lens. In some embodiments, a fetal tissue product disclosed, a device, or a portion of a device disclosed may be delivered to a surface of an eye using an index finger, or any known methods or technologies, or techniques.

### Methods of Treatment

The fetal tissue product or the AM products described herein may be used in a variety of applications where sterility and adhesiveness are desirable. For example, the sheets described herein may adhere well and provide a treatment for wounds, burns, and surgical incisions. Further, the fetal tissue product or the AM products described herein may be administered directly to the skin and covered with a suitable dressing, or wrapped around or incorporated into a stent, scaffold, or implant for placement in a tissue in need of healing. The size and adhesiveness of the sheet products make them ideal for placement and healing of the fingers, toes, nose, ears, genitals, knees, ankles, elbows, etc.

Disclosed herein in some embodiments is a method of treating a disease or disorder of an eye of an individual in need thereof. The sterile and adhesive properties of the fetal tissue and Am products make them ideal to treat a disease or disorder of the eye, tear ducts, or eyelids. In some embodiments, a method of treating a disease or disorder of an eye comprises administering a fetal tissue product disclosed herein.

A fetal tissue product or an AM product described herein may be used alone as a stand-alone product e.g., a therapeutic or it can be used in combination with any other suitable product(s) or therapeutic(s).

In some embodiments, a method of treating a disease or disorder of an eye comprises administering an ophthalmic device comprising a fetal tissue product disclosed herein. In some embodiments, a method of treating a disease or disorder of an eye comprises administering an ophthalmic device comprising a fetal tissue product adhered to a surface of an eye. In some embodiments, a method of treating a disease or disorder of an eye comprises administering an ophthalmic device comprising a fetal tissue product adhered to a contact lens and administering or placing the lens on directly to a surface of an eye of an individual in need thereof. In some embodiments, a method of treating a disease or disorder of an eye comprises administering a fetal tissue product disclosed comprising an amniotic membrane (AM) product. In some embodiments, a method of treatment comprises placing an AM product on a surface of an eye e.g., a corneal surface. In some embodiments, a method of treatment comprises administering a fetal tissue product disclosed by adhering it to a surface of an artificial contact lens. In some embodiments, a fetal tissue product disclosed may be adhered to an artificial contact lens and then placed on a surface of the eye of the individual, e.g., on a corneal surface. In some embodiments, a method of treatment comprises administering a fetal tissue product e.g., an AM product to treat a disease or disorder of an eye. In some embodiments, a disease or disorder of an eye comprises dry-eye-disease (DED). In some embodiments, a disease or disorder of an eye comprises corneal abrasion. In some embodiments, a disease or disorder of an eye comprises superficial punctate keratitis. In some embodiments, a disease or disorder of an eye comprises non-healing injury/wound. In some embodiments, a disease or disorder of an eye comprises post debridement condition. In some embodiments, a disease or disorder of an eye comprises epithelial basement membrane dystrophy condition. In some embodiments, a disease or disorder of an eye comprises neurotrophic keratitis. In some embodiments, a disease or disorder of an eye comprises corneal epithelial defect. In some embodiments, a disease or disorder of an eye comprises corneal ulcer. In some embodiments, a disease or disorder of an eye comprises recurrent corneal epithelial erosion. In some embodiments, a method of treatment is targeted to reduce one or more adverse events from happening to an eye of an individual in need thereof. In some embodiments, a method of treating an eye disease or disorder of an eye comprises reducing one or more adverse events may comprise reducing ocular ulcers or infiltrates. In some embodiments, the AM product disclosed may be used to treat and reduce adverse incidence rates. In some embodiments, a method of treating a disease or condition of an eye can produce improvement in a disease or condition of an eye of a subject. In certain embodiments, the disease or disorder of the eye comprises Stevens-Johnson syndrome, glaucoma, macular hole, retinal detachment, macular degeneration or oculoplastic applications.

The fetal tissue products and the AM products currently described can be administered to an eye of an individual in need thereof. The administration can be to the surface of the eye, to the intravitreal space of the eye, the retina of the eye, the macula of the eye, the tear duct of the eye, or the conjunctiva of the eye. In certain embodiments, administration is to the surface of the eye. In certain embodiments, administration is to the intravitreal or subretinal space of the eye. In certain embodiments, administration is to the macula of the eye. In certain embodiments, administration is to the retina of the eye. In certain embodiments, administration is to a tear duct of the eye. In certain embodiments, administered to an intravitreal, epiretinal or subretinal space of the eye. In certain embodiments, the administration is during glaucoma surgery or oculoplastic surgeries the eye.

In some embodiments, a treatment described herein e.g., a fetal tissue product or AM product may include second agent or an additional therapeutic agent. For example, a treatment described herein e.g., a fetal tissue product or AM product may further include a second agent or an additional therapeutic agent comprising e.g., artificial tears, an antibiotic, or both. In some embodiments, an antibiotic may comprise one or more antibiotics. In some embodiments, one or more antibiotics comprise any known antibiotic agents. For example, one or more antibiotics may comprise any known antibiotics used to treat ocular diseases or conditions. In some embodiments, the second or additional therapeutic agent(s) may be administered concurrently (e.g., simultaneously, essentially simultaneously or within the same treatment protocol. In some embodiments, a treatment protocol comprises a fetal tissue product, or AM product, and such a protocol may also include a second agents or an additional therapeutic agent(s) disclosed herein or necessary for treatment of an ocular disease or condition. In some embodiments, administration of artificial tears or one or more antibiotics may reduce the rate or incidence of adverse events (AEs) in the eyes. In some embodiments, administering artificial tears or one or more antibiotics, or a combination thereof, to an eye may reduce the risk of developing at least one adverse event (AE). some embodiments, one or more antibiotics administered may comprise, ciprofloxacin, erythromycin, tobramycin, polymyxin B and trimethoprim, levofloxacin, neomycin, polymyxin B, dexamethasone or a combination thereof. In some embodiments, any other suitable ophthalmic antibiotics may be administered to an individual in need thereof. In some embodiments, artificial tears or one or more antibiotics may be administered to the surface of an eye e.g., surface of a corneal, or to a substrate described herein. In some embodiments, a method of treatment comprising placing a fetal tissue product or ophthalmic device to an eye can comprise placing the tissue product or device for at least 1 hour or more, 1 day or more, 2 days, 3 days, 4 days, 5 days, 6 days, one week, two weeks or more on an eye of an individual.

The AM treatment duration administered to a surface of an eye is dependent in part on the severity of the eye condition of the individual being treated. In instances where an AM product comprising HC-HA/PTX3 disclosed is administered to a human subject. In some embodiments, the AM product administered on the surface of an eye can be by unit or a dosage. In some embodiments, the size of the tissue product for example, can normally be determined by the prescribing physician. In some embodiments the dosage is known to generally varying according to the age, sex, diet, weight, general health and response of the individual. In some embodiments, the unit, size of AM product, or dosage can vary by the severity of the individual's symptoms. In some embodiments, the unit or size of AM product can vary by the precise disease or condition being treated, the severity of the disease or condition being treated, time of administration, at the discretion of the prescribing physician.

In some embodiments, an AM product comprising HC-HA/PTX3 can be a packaged composition. An AM product package composition can be a unit dosage form suitable for single administration. In some embodiments, an AM product or composition described herein is administered in a single dose or unit usage. In unit dosage form, the AM product can be provided on 1 day, 2 or more days or at weekly, biweekly, monthly, bi-monthly etcetera as appropriately required to provide sufficient quantities needed as determined by a licensed medical physician, or licensed medical personnel. In some embodiments, the unit dosage is in the form of a package containing discrete quantities of an AM product. Non-limiting examples are packaged AM products sealed for single dose containers. In some embodiments, multiple dose re-sealable containers are used, in which case it is typical to include a preservative in an AM composition.

In some embodiments, in cases where the individual's status does improve, upon the medical doctor's discretion, an AM product disclosed herein is administered continuously or discontinued for a certain length of time (i.e., an “AM product holiday”). In some embodiments, the length of the holiday varies between 2 days and 1 year, including by way of example only, 1 day, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days.

Once improvement of the individual's condition has occurred, a maintenance dose is administered if necessary. In some embodiments, subsequently, the dosage or the frequency of administration, or both, is reduced, as a function of the symptoms, to a level at which the improved disease, disorder or condition is retained. In some embodiments, individuals require intermittent treatment on a long-term basis upon any recurrence of symptoms.

In some embodiments, the composition described herein is packaged as articles of manufacture containing packaging material, a composition which is effective for prophylaxis and/or treating an eye indication, disease, disorder, condition, or a combination thereof. The AM product disclosed can comprise a label that indicates that the composition is to be used on the surface of an eye. In some embodiments, an AM product can be packaged in unit dosage forms contain an amount of the composition for a single use, single-dose, or multiple doses. In some embodiments, the packaged compositions contain an AM product which can be packaged hydrated or not dehydrated and contain for example an isotonic solution (e.g., sodium chloride or saline) prior to administration. The AM product can further comprise a preservative.

In the case wherein the individual's condition does not improve, upon the doctor's discretion the fetal tissue products or AM products described herein is administered chronically, that is, for an extended period of time, including throughout the duration of the individual's life in order to ameliorate or otherwise control or limit the symptoms of the individual's disease or condition.

The fetal tissue products or AM products described herein can be administered before, during, or after an eye surgery to aid in healing. Such surgeries may comprise retina surgery, corneal surgery, glaucoma surgery, vision correction surgery, conjunctivodacryocystorhinostomy (CDCR) surgery, dacryocystorhinostomy (DCR) surgery, cataract surgery, corneal transplant, laser-assisted in situ keratotomy (LASIK), photorefractive keratectomy, vitrectomy, refractive surgery, refractive lens exchange, intrastromal corneal ring segment surgery, small incision lenticule extraction, trabeculectomy surgery, oculoplastic surgery. The administration can be to the surface of the eye, to the intravitreal space of the eye, the retina of the eye, the macula of the eye, the tear duct of the eye, or the conjunctiva of the eye. The fetal tissue products can be administered to a tear duct of the eye using a lacrimal intubation tube or shunt, including without limitation a Jone's tube or a Crawford tube.

The fetal tissue products or AM products described herein can be administered to an eye that has experienced trauma such as an abrasion a laceration, or a burn. In certain embodiments, the burn is a chemical burn or a thermal burn.

Exemplary procedure to administer fetal tissue product or AM product: Anesthetize the eye and apply a drop of anesthetic prior to placement. In some embodiments, placing anesthetic reduces the potential discomfort associated with the low pH of a contact lens disclosed e.g., a collagen shield (CS). Antibiotic Drops: A prophylactic antibiotic drop on an ocular surface prior to placement is recommended. Placement & Hydration: Place the prepared contact lens and AM product on an ocular surface. In some embodiments, a contact lens comprises a lens disclosed herein. Once placed, apply 2-3 drops of saline to ensure complete hydration of a contact lens. Examine for and address any air bubbles after insertion. Taping Methods: After placement, use one of the following methods: Patch (Total Closure), Nictavi Patch (Partial Closure), Tape Tarsorrhaphy (Partial Closure). Taping Guidance: The treated eye should remain taped for 48-72 hours to retain a contact lens disclosed as it dissolves. When removing a tape, do so gently to avoid excessive pulling on an eyelid, as a contact lens disclosed/tissue could fall out. Preservative-Free Artificial Tears: If using a Nictavi Patch or Tape Tarsorrhaphy, a patient should apply preservative-free artificial tears at least 3 times daily, during treatment. Steroid Use: The use of a concomitant steroid during treatment is not necessary or preferred. Post-insertion Evaluation: Ensure proper centration of contact lens disclosed and AM product over cornea. Allergies and warning: Patient may experience a temporary stinging sensation following an insertion of a contact lens disclosed which should neutralize quickly. This application method is not suitable if a patient has a known allergy to collagen or bovine products. A contact lens e.g., contact lens disclosed wear time should not exceed 72 hours. Prolonged wear can result in bacterial or fungal infections. If a patient presents with any Defects, Ulcers, EBMD or RCE this product may not be suitable.

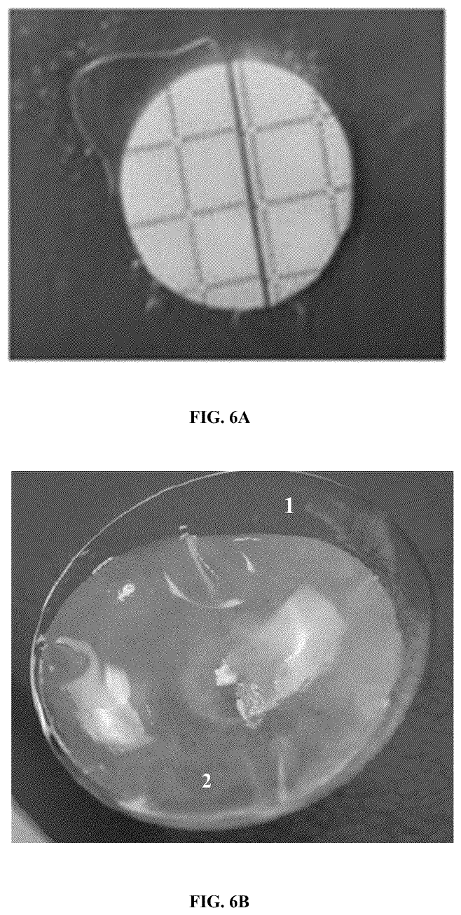

Placing the fetal tissue product or AM product onto a bandage contact lens surface: As disclosed, a fetal tissue product or an AM product disclosed is intended to be used on ocular surfaces. In some embodiments, an AM product is placed on the ocular surface to prevent or treatment for ocular surface indications. In some embodiments, a contact lens e.g., a Night and Day Bandage contact lens (BCL) can be adhered to an AM product and strongly adhere to an inner concave surface of BCL for at least 3 or more days without falling out. Illustrated herein are directions to press the fetal tissue product or AM product onto an exemplary contact lens, the bandage contact less, is demonstrated in steps 1) open the AM product package to retrieve the product (FIG. 5A), 2) place AM product onto the surgical tray surface (FIG. 5B), 3) use forceps to remove the outer packaging, to access the AM product in the inner pouch and press onto the lens (FIG. 5C), 4) retrieve and access the contact lens (BCL; FIG. 5D), 5) adhere inverted BCL onto the AM product (FIG. 5E), and 6) revert the BCL or a contact lens to its normal shape once the fetal tissue product or AM product has been spread onto the lens (FIG. 5F). FIG. 6A shows a fetal tissue product or AM product after unpackaging. FIG. 6B shows AM product (2) after it is placed a surface of a contact lens (1). A contact lens is placed on an AM product or on a surface of an eye and will remain on the surface without sutures. As mentioned above, the AM product contains adhesive properties on both the epithelial and stroma sides such that it can adhere to a surface of the eye or corneal without sutures and remains there for at least 3 days or more until treatment is complete or until an AM tissue product has dissolved. In some embodiments, an AM product can be directly applied to an ocular surface instead and then adhering with a gentle force to stick it in place once lodged into the ocular surface. Any method can be used to place an AM product on directly on an ocular surface including for example, use of an index finger. Any method or technology that is useful for placing a contact lens on an ocular surface can be utilized if it is aseptically managed so there is no infection of an eye. As seen in FIGS. 5F and 6B, the lens contacted only by the adhesiveness of the AM product is ready to be placed on the corneal surface using the same technique for inserting a contact lens using an index finger. The thickness for both fresh and cryopreserved fetal tissue products can be any thickness that allows the product to be comfortably placed. Non-limiting ranges include from about 5 μm to about 50 μm, 10 μm to about 30 μm, 15 μm to about 25 μm, 20 μm to about 80 μm, 50 μm to about 100 μm, 75 μm to about 150 μm, and about 500 μm to about 900 μm, or 700 μm to about 1000 μm.

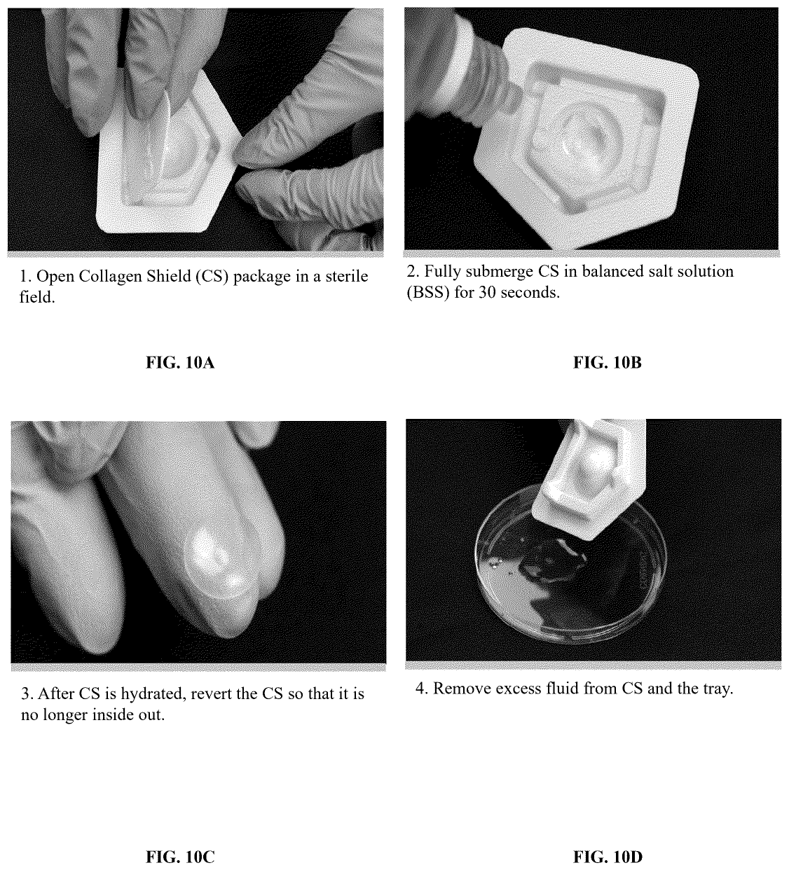

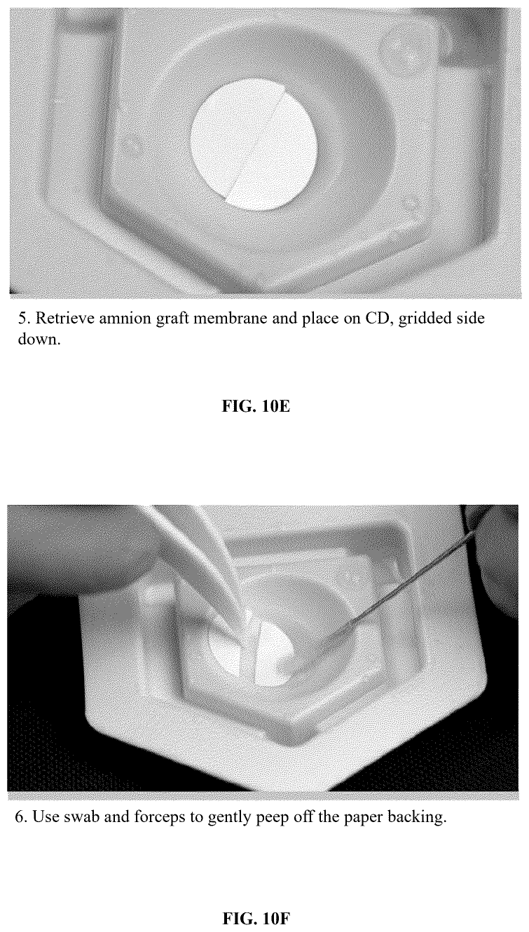

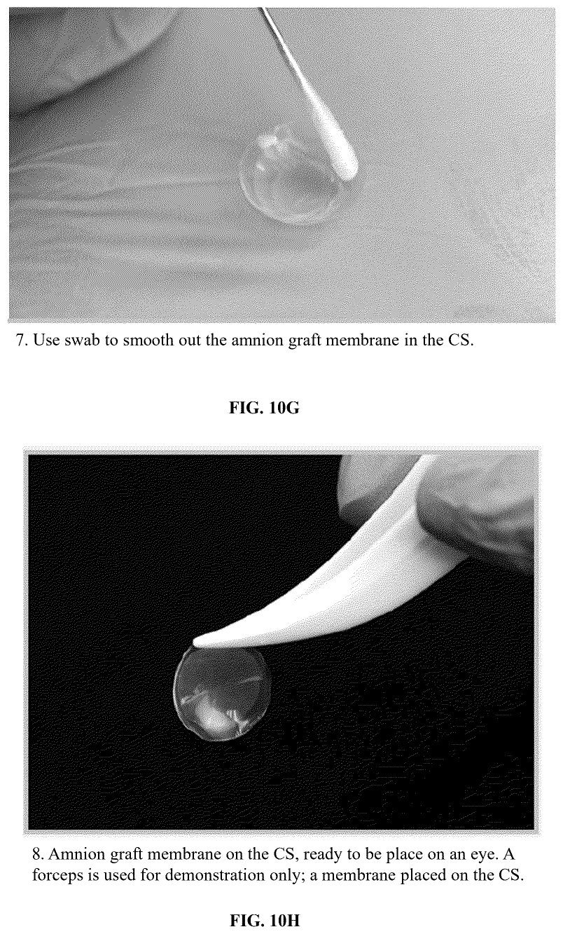

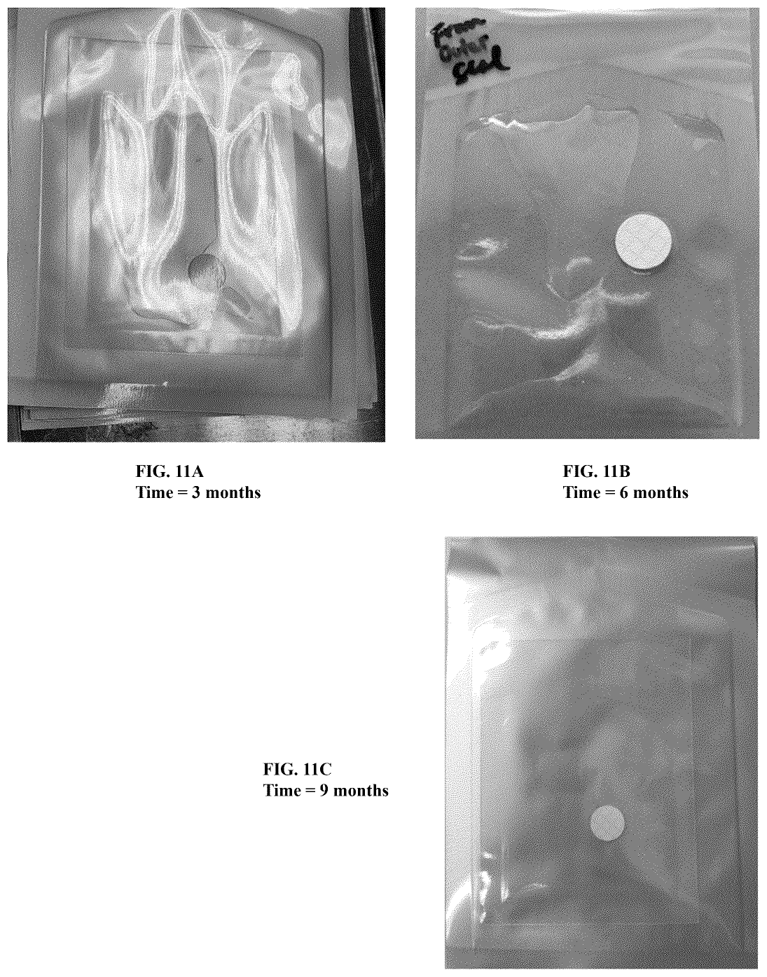

Placing the fetal tissue product or AM product on a collagen corneal shield or collagen shield lens (CS): Steps and guidelines showing the process of how to aseptically unpack and place a fetal tissue product or an AM product of the present disclosure onto a contact lens e.g., a collagen shield (CS) contact lens, are illustrated in FIG. 10A-FIG. 10H. Directions to open and press an AM product onto an exemplary contact lens are similar to those used in the BCL above. The steps include: 1) open a contact lens disclosed package e.g., a collagen shield (CS) package in a sterile container (FIG. 10A), 2) fully submerge a contact lens disclosed in balanced salt solution (BSS) for 30 seconds (FIG. 10B), 3) after contact lens disclosed is hydrated, revert the contact lens disclosed so that it is no longer inside out (FIG. 10C), 4) remove excess fluid from the contact lens disclosed and the tray (FIG. 10D), 5) retrieve amnion graft membrane and place on CD, gridded side down (FIG. 10E), 6) use swab and forceps to gently peep off the paper backing (FIG. 10F), 7) use swab to smooth out the amnion graft membrane in the contact lens disclosed (FIG. 10G), 8) amnion graft membrane on the contact lens disclosed (FIG. 10H) and place on an eye using known methods of placing contact lens on the surface of an eye e.g., using an index finger. The process of adhering an AM product to a surface of an eye or any contact lens can suitably be conducted at the physician's office.

### Method of Manufacturing

Disclosed herein in some embodiments is a method of manufacturing or making a fetal tissue product or amniotic membrane (AM) product disclosed. In some embodiments, a method of making a fetal tissue product. In some embodiments, a method of making a fetal tissue product comprises obtaining a fetal tissue, subjecting the fetal tissue product to cryopreservation, terminal sterilization, isolating and recovering the fetal tissue under conditions sufficient to isolate the fetal tissue product disclosed herein.

An AM product can be processed by the methods described alone or in combination with any other methods of preparation or processing, etcetera.

### Preparation of Amniotic Membrane (AM) Products for Characterization

A fetal tissue product disclosed can be generated using various methods or generated by a method disclosed in combination with any other method(s). The method provided here is illustrative. In some embodiments, a fetal tissue product refers to the amniotic membrane (AM) product described herein. In some embodiments, a method provided comprises a fetal tissue sample or amniotic membrane (AM) sample prepared by a certain cryopreservation method of making the steps of which are described in U.S. Pat. No. 9,682,160, which is herein incorporated by reference in its entirety. In some embodiments, a certain cryopreservation method comprises a cryopreservation method A. In some embodiments, AM samples used for characterization may be prepared by a cryopreservation method A as described herein, the method used alone, or in combination with any other method(s). In some embodiments, a cryopreservation method A comprises processing of an AM tissue aseptically performed under Class II Biological Safety Cabinet using minimal manipulation of an AM tissue. Briefly, frozen placenta may be thawed under controlled conditions and an amniotic membrane (AM) is isolated from a placenta. An AM is further separated from a chorion. The AM is then subjected to a series of steps: 1) Wash with saline; 2) Gently remove blood from stromal side of AM; 3) Wash with saline (Soak+Gentle Massage Optional (if blood still present)); 4) Repeat wash until tissue is cleaned to its entirety; 5) Wash with saline, Swirl and let sit—1 min; 6) Wash saline Swirl and let sit—1 min; 7) Wash with saline—Let Sit; 8) Wash with saline. AM is then placed on sterile membrane, e.g., a polyethersulfone (PES) membrane, with stromal side down and a tissue graft is cut according to size. Tissue grafts are then placed in pouches filled with storage solution (saline or isotonic solution) and sealed. Products are kept in a sub-zero freezing temperature e.g., in a freezer (−20° C.) until needed e.g., to process and prepare a fetal tissue product or an AM product. A gamma irradiation sterilization process for medical devices is utilized which applies to i) a sterilization dose of 25 kGy, b) establishment of a radiation sterilization dose which is primarily set on bioburden (i.e., initial contamination that describes a population of active pathogens before sterilization). The optimal radiation dosage 25 kGy is routinely used in many tissue banks.

In some embodiments, an AM product described herein is not a dehydrated product. In some embodiments, an AM product described herein comprises a hydrated product. In some embodiments, an AM product described herein comprises an example of a cryopreserved AM that undergoes terminal sterilization to enable room temperature storage as a sterile product with sterility assurance level of 10-6. An example of an AM product disclosed is the CAM360® AmnioGraft™ product disclosed herein.

SteriTek® preservation process, and/or a CryoTek® technology: In non-limiting examples, in some embodiments, a method of making a fetal tissue product may comprise a SteriTek® preservation process, and/or a CryoTek® technology (technology is disclosed herein. of BioTissue®). In some embodiments, a method of making a fetal tissue product may comprise a SteriTek® preservation processing of an amniotic membrane (AM) tissue preservation. a method of making a fetal tissue product may comprise a process that may yield a shelf-stable product while maintaining a natural hydration of a tissue product. In some embodiments, a fetal tissue e.g., an amniotic membrane may be cleaned, processes and packaged consistent with a preservation process that may maintain the structural and biological integrity of a natural tissue. In some embodiments, such a processing method may comprise SteriTek® preservation process. In some embodiments, a preservation process that may maintain the structural and biological integrity of a natural tissue may include e.g., a CryoTek® cryopreservation process. In some embodiments, a fetal tissue e.g., AM tissue processed by CryoTek cryopreservation process may then be terminally sterilized e.g., under gamma irradiation. In some embodiments, processing AM under terminal sterilization, e.g., under gamma irradiation may yield a fully hydrated AM product that is stable at room temperature e.g., under a controlled room temperature. In some embodiments, the method comprises sterilizing and packaging a fetal tissue product of the present disclosure.

Sterilization and packaging: In some embodiments, sterilizing a fetal tissue product or AM product can be processed using methods disclosed above, e.g., using a SteriTek® preservation process and a CryoTek® cryopreservation process (technologies of BioTissue®). In some embodiments, a method of manufacturing a fetal tissue product comprises obtaining donated placenta or birth tissue according to Current Good Tissue Practice (CGTP) and Current Good Manufacturing Practice (CGMP) regulations established by the U.S. Food and Drug Administration (FDA). In some embodiments, a method of making a fetal tissue product disclosed comprises terminally sterilizing a fetal tissue product under conditions to allow a fetal tissue product in a hydrated state (not dehydrated). In some embodiments, a fetal tissue product can be sterilized under icy conditions below 0° C. In some embodiments, a fetal tissue product or AM product disclosed may be cryopreserved and aseptically processed, cell devitalized and terminally sterilized via gamma irradiation with a Sterility Assurance Level (SAL) of 10-6 (ISO 11137; VDmax25 Gamma Radiation and validated according to inhouse established protocols approved by a regulatory body, e.g., FDA. Such protocols include for example protocol for Amniotic Membrane (AM) and Umbilical Cord (UC) Graft Family. In some embodiments, a fetal tissue product or AM product may be stored in a pouch packaging. In some embodiments, packaging comprises an inner and outer pouch packaging as well as a membrane, e.g., a polyethersulfone (PES) polymer backing. In some embodiments, an inner and outer pouch packaging as well as polyethersulfone (PES) polymer backing are sterilized via gamma irradiation according to ISO 11137 prior to use. In some embodiments, a final fetal tissue product or AM product may not be re-sterilized or autoclaved before use. In some embodiments, a fetal tissue product or AM product in the pouch may be stored in saline (200 μl of 0.9% w/v sodium chloride (NaCl) solution) and be delivered on a non-implantable, gridded backing (e.g., a Polyethersulfone backing, also known as PES backing) for easier handling and application. In some embodiments, a gridded backing with a slit in the middle aids in the detachment of a fetal tissue product or AM product. In some embodiments, a fetal tissue product or AM product may be manufactured and processed without a backing member. In some embodiments, any of the steps disclosed herein for manufacturing a fetal tissue product disclosed may be skipped. any of the steps disclosed herein for manufacturing a fetal tissue product disclosed may be mandatory. In some embodiments a fetal tissue product or AM product packaging comprises 200 μl saline storage solution to prevent a fetal tissue product or AM tissue product from drying out during storage. In some embodiments, use of saline comprises any amount of saline. In some embodiments, use of saline is not mandatory or required if at all conditions for producing a hydrated sterile fetal tissue product are available.

Several methods of making a fetal tissue product or AM product disclosed including placing it onto an artificial contact lens are already provided in several other sections of the application e.g., in the example, or drawing sections and will not be repeated in much detail.

Several methods describing a fetal tissue product or AM product disclosed including methods of preparation and how to characterize a fetal tissue product or AM product are provided in other sections of this application and will not be repeated here in much detail. In some embodiments, such methods of manufacturing and generating, characterizing a fetal tissue product or AM product to ensure it confirms to biological characteristics of a tissue product disclosed are provided in detail in the examples and in the figures and elsewhere throughout this application. In some embodiments, these characteristics include biological activities e.g., comprising HC-HA/PTX3 complex and individual macromolecules.

In some embodiments, a fetal tissue product disclosed can be generated using various methods disclosed herein e.g., in example section or drawings. In some embodiments, a method provided may be used to prepare, generate, or process a fetal tissue sample or AM tissue sample, a fetal tissue product or Am product. In some embodiments, a cryopreservation method described herein may be used to prepare, generate, or process an AM tissue sample disclosed, or an AM tissue product disclosed herein. In some embodiments, a cryopreservation method A may be used to prepare, generate, or process an AM tissue sample or an AM tissue product described herein. In some embodiments, a fetal tissue is processed aseptically under Class II Biological Safety Cabinet using minimal manipulation of the tissue. In some embodiments, frozen placenta may be thawed under controlled conditions and a fetal tissue e.g., an amniotic membrane (AM) may be isolated from a placenta. In some embodiments, a fetal tissue or AM sample may be separated from chorion then subjected to a series of steps: 1) Wash with saline; 2) Gently remove blood from stromal side of AM; 3) Wash with saline (Soak+Gentle Massage Optional (if blood still present)); 4) Repeat wash until tissue is cleaned to its entirety (e.g., is translucent or clear); 5) Wash with saline, Swirl and let sit—1 min; 6) Wash saline Swirl and let sit to 1 min; 7) Wash with saline—Let Sit; 8) Wash with saline. AM was then placed on sterile membrane, e.g., a polyethersulfone (PES) membrane, with stromal side down and the tissue graft was cut according to size. The tissue grafts may then be placed in pouches filled with storage solution (e.g., saline or isotonic solution) and sealed. In some embodiments, a fetal tissue or AM product may be kept in a freezer (−20° C.) until needed e.g., to process and prepare the fetal tissue product or the AM product. In some embodiments, a gamma irradiation sterilization process for medical devices may be utilized or applied at (a) a sterilization dose of 25 kGy, (b) a radiation sterilization dose set on bioburden (i.e. initial contamination that described population of active pathogens before sterilization). In some embodiments, an optimal radiation dosage 25 kGy may be used as routinely used in many tissue banks.

### Kits/Articles of Manufacture

Disclosed herein in some embodiments is a kit for treating a disease or disorder of an eye. In some embodiments, a kit comprises a fetal tissue product or AM product disclosed herein. In some embodiments, a kit comprises a fetal tissue product disclosed and one or more substrates comprising artificial contact lenses, and optionally, saline or buffer, or any one or more of tweezers, swabs, forceps, or surgical trays. In some embodiments, a kit comprising a fetal tissue product that is adhered to a backing material. In some embodiments, a backing material comprises a polyethersulfone (PES) polymer. In some embodiments, a backing material may be gridded. a backing material. In some embodiments, a kit comprises a fetal tissue product that may be stored in an aqueous solution. In some embodiments, an aqueous solution may comprise a solution comprising a 0.9% saline solution. In some embodiments, a kit comprises a fetal tissue product for treating a disease or disorder of an eye comprising a dry eye disease. In some embodiments, a kit comprises a fetal tissue product for treating a disease or disorder of an eye comprising a corneal abrasion. In some embodiments, a kit comprises a fetal tissue product for treating a disease or disorder of an eye comprising superficial punctate keratitis. In some embodiments, a kit comprises a fetal tissue product for treating a disease or disorder of an eye comprising a non-healing injury/wound. In some embodiments, a kit comprises a fetal tissue product for treating a disease or disorder of an eye comprising a post debridement. In some embodiments, a kit comprises a fetal tissue product for treating a disease or disorder of an eye comprising an epithelial basement membrane dystrophy. In some embodiments, a kit comprises a fetal tissue product for treating a disease or disorder of an eye comprising neurotrophic keratitis. In some embodiments, a kit comprises a fetal tissue product for treating a disease or disorder of an eye comprising reduced corneal sensitivity. In some embodiments, a kit comprises a fetal tissue product for treating a disease or disorder of an eye comprising a corneal epithelial defect. In some embodiments, a kit comprises a fetal tissue product for treating a disease or disorder of an eye comprising a corneal ulcer. In some embodiments, a kit comprises a fetal tissue product for treating a disease or disorder of an eye comprising recurrent corneal epithelial erosion. In some embodiments, a kit comprises a fetal tissue product for treating a disease or disorder of an eye comprising a dry eye disease. In some embodiments, a kit comprises a fetal tissue product for treating a disease or disorder of an eye comprising superficial punctate keratitis. In some embodiments, a kit comprises a fetal tissue product for treating a disease or disorder of an eye comprising neurotrophic keratitis. In some embodiments, a kit comprises a fetal tissue product one or more substrates. In some embodiments, one or more substrates comprise a bandage contact lens (BCL), a collagen shield (CS) or a collagen corneal shield. In some embodiments, one or more substrates comprise a soft contact lens. In some embodiments, a soft contact lens comprises any known soft contact lens; non-limiting examples of a soft contact lens include, comfilcon A, senofilcon A, balafilcon A, methafilcon A, nelfilcon A hydrogel or silicon hydrogel.

Kit supplies: In some embodiments, a kit disclosed herein comprises a fetal tissue product, or AM product disclosed. In some embodiments, a fetal tissue product, or AM product. In some embodiments, a kit comprises a 12 mm Sterile fetal tissue product or AM product disclosed. In some embodiments, a kit comprises a collagen shield (e.g., SOFT SHIELD®). In some embodiments, a kit comprises a Balanced Salt Solution (BSS) such as 0.9% saline. In some embodiments, a kit comprises a Sterile Forceps. In some embodiments, a kit comprises a Sterile Polyester Tipped Applicator Swab.

In some embodiments, a kit described herein may comprise a second agent or an additional therapeutic agent. In some embodiments, a second agent or an additional therapeutic agent may comprise one or more agents. In some embodiments, a second agent or an additional therapeutic agent may comprise artificial tears. In some embodiments, artificial tears administered may comprise any known sterile artificial tears. In some embodiments, a second agent or an additional therapeutic agent comprises an antibiotic. In some embodiments, a second agent or an additional therapeutic agent comprise one or more antibiotics. In some embodiments, a second agent or an additional therapeutic agent may comprise one or more agents, e.g., artificial tears, one or more antibiotics, or both artificial tears and any number of antibiotics. In some embodiments, administration of artificial tears or one or more antibiotics may reduce the rate or incidence of adverse events in the eyes (AEs). In some embodiments, one or more antibiotics administered may comprise, ciprofloxacin, erythromycin, tobramycin, polymyxin B and trimethoprim, levofloxacin, neomycin, polymyxin B, dexamethasone or a combination thereof. In some embodiments, any other suitable ophthalmic antibiotics may be administered to an individual in need thereof. In some embodiments, an artificial tear or antibiotic e.g., one or more antibiotics may be administered as an additional therapeutic agent(s). In some embodiments, an additional therapeutic agent may be administered concurrently with administration of a fetal tissue product or AM product (e.g., simultaneously, essentially simultaneously or within the same treatment protocol.

For use in the therapeutic applications of the fetal tissue products or AM products described herein, kits and articles of manufacture are also described herein for the prevention, repair, or treatment of eye indications disclosed herein e.g., ocular surface disorders, conditions, disorders, diseases, or a combination thereof. Such kits can include a carrier, package or a container that is compartmentalized to receive one or more containers such as vials, forceps, swabs, and the like, each of the container(s) including one of the separate elements to be used in a method described herein. Suitable containers include, for example, bottles, vials, sterile swabs, sterile forceps, absorbent towels, or any combination thereof. The containers can be formed from a variety of materials such as glass, plastic, wood.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, bottles, and any packaging material suitable for a selected formulation and intended mode of administration and treatment. A wide array of formulations of the compounds and compositions provided herein are contemplated as a variety of treatments for disclosed disease, disorder, or condition.

For example, the container(s) can include one or more AM products described herein, alone or in combination with another agent. Such kits optionally comprising a compound with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of the compositions described herein. Non-limiting examples of such sterile materials include, but not limited to buffers, diluents, filters, forceps, swabs; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded, or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, AM products or compositions can be presented in a pack or dispenser device which can contain one or more-unit dosage forms containing a compound provided herein. The pack can for example contain metal or plastic foil, such as a blister pack which contains the AM product, for example. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible 1 carrier can also be prepared, placed in an appropriate container, and labeled for treatment of an indicated condition.

In some embodiments, disclosed herein is a kit for treating a disease or disorder of an eye. In some embodiments, a kit can comprise a fetal support tissue product and any one or more of a tweezers, a swab, and a surgical tray. In some embodiments, a kit can comprise a fetal support tissue that is adhered to a backing material. In some embodiments, a backing material can be a polyethersulfone (PES) polymer. In some embodiments, a backing material can be gridded. In some embodiments, a kit can comprise a fetal support tissue for treating a disease or a disorder of an eye.

The compositions, methods, kits, or devices disclosed herein can be administered to a subject in need thereof to prevent or treat any known eye indication before or after surgery. For example, the present disclosure can be used pre-operatively or post-operatively. In certain embodiments, an AM product can be applied to corneal transplantation. In certain embodiments, an AM product disclosed can be applied on a corneal indication or condition which can occur post operative or post corneal transplantation. In certain embodiments, a corneal indication can develop in a corneal but not require corneal transplant.

The compositions, methods, kits, devices (e.g., tissue grafts and tissue-derived biodegradable compositions) disclosed herein (e.g., fetal tissue product/extract or amniotic membrane (M) product) can be used for their healing properties in promoting the prevention, repair, regeneration, rejuvenation, healing, treatment, or any combination thereof, of an eye indication. In some embodiments, an eye indication can comprise a dry eye disease, disorder, or condition. There are several dry eye disease conditions, disorders, or diseases. The present disclosure can be used for any dry eye disease, disorder, or condition. Non-limiting examples of eye or ocular indications can include dry eye disease (DED), neurotrophic keratitis, corneal epithelial defect, corneal ulcer, recurrent corneal epithelial erosion, Superficial keratitis, punctate epithelial keratitis, keratitis, punctate epithelial erosion, epithelial basement membrane dystrophy, corneal ulcers (infectious or not infectious).

The present compositions or devices can be used to treat any other eye indication. In certain embodiments, providing the present disclosed composition, formulations, can lead to a treatment, a prophylaxis, a preventative measure of an eye indication. In certain embodiments, administration of the present disclosure can be targeted to one, two, three or more conditions, infections, symptoms, diseases, syndromes in a subject in need thereof. Other indications can include keratoconus, bullous keratopathy, failed grafts, non-herpetic corneal scars, corneal stromal dystrophies, trauma, thin descemetocele, pellucid marginal degeneration, corneal scar, endothelial corneal dystrophies, anterior segment dysgenesis, corneal ectasia which can occur after laser in situ keratomileusis, corneal fibrosis, or any other corneal-related condition.

### Applications

In some embodiments, disclosed herein, is a method of treating a disease or a disorder of an eye where the disease or the disorder of the eye can comprise a dry eye, a corneal abrasion, a neurotrophic keratitis, a corneal epithelial defect, a corneal ulcer, or a recurrent corneal epithelial erosion. In some embodiments, a method of treating a disease or a disorder of an eye can comprise dry eye disease. In some embodiments, a method of treating a disease or a disorder of an eye can comprise a corneal abrasion. In some embodiments, a method of making a treatment for a disease of an eye can comprise contacting a fetal support tissue product to a surface of a contact lens, thereby adhering the fetal tissue product to the contact lens. In some embodiments, a disease or a disorder of an eye can comprise dry eye disease. In some embodiments, a disease or a disorder of an eye can comprise a corneal abrasion. In some embodiments, a disease or disorder of an eye can comprise a dry eye, a corneal abrasion, neurotrophic keratitis, a corneal epithelial defect, a corneal ulcer, or a recurrent corneal epithelial erosion.

The present disclosure can be used on various eye or ocular indications. In some embodiments, an eye or ocular indication can comprise an inflammation on a surface of an eye. In some embodiments, an eye indication can comprise a conjunctiva indication or disorder, a corneal indication or disorder. An eye condition, disease or disorder can occur on the surface of an eye. In some embodiments, an eye indication can be a corneal indication e.g., a corneal disorder, corneal condition, or corneal infection. In certain embodiments, corneal disorder, condition, or infection can be a corneal ulcer. The corneal ulcer can be infectious. In certain embodiments, the corneal ulcer is not infectious. In certain embodiments, the present disclosure can be used to promote prevention, repair, healing, or treatment on the surface of an ocular surface of an eye.

The present compositions or devices can be used to treat an ocular surface indication e.g., a corneal indication to provide relief or treatment. In certain embodiments, provisions or administering the present disclosure can be a curative treatment for a corneal indication. The provision or administration of the present disclosed composition or devices can alleviate symptoms, or bodily stress to the subject in need thereof. The subject can be treated wherein the administration of the claimed disclosures leads to at least one or more corneal-related indications. Provision of the present disclosure can lead to reduction in a symptom, a disorder, a condition, a syndrome, a disease, an infection of a corneal indication, disease, disorder, or condition. Administration of the present disclosure can be targeted to a particular condition such that it improves, alleviates, or reduces pain or symptoms. In certain embodiments, use of the present disclosure can bring about healing or improve a condition in such a way to make it different from before the administering of an AM product disclosed herein. An AM product disclosed can be used for example, in the prevention, repair, healing, rejuvenation, regeneration of other ocular indications. In some embodiments, an AM product can be targeted to an ocular indication to prevent further damage or heal an ocular tissue or treat an ocular surface to e.g., alleviate pain, promote prevention, healing, or repair of an ocular condition following injury, trauma, disorder, condition, disease, or infection.

### Embodiments 1

Embodiment 1 comprises a fetal tissue product comprising fetal tissue, wherein the fetal tissue product comprises less than about 30 micrograms of hyaluronic acid (HA) per 32 cm².

Embodiment 2 comprises a fetal tissue product of embodiment 1, wherein the fetal tissue is a sheet.

Embodiment 3 comprises a fetal tissue product of embodiment 1 or 2, wherein the fetal tissue product comprises less than about 20 micrograms of hyaluronic acid per 32 cm².

Embodiment 4 comprises a fetal tissue product of embodiment 1 or 2, wherein the fetal tissue product comprises less than about 10 micrograms of hyaluronic acid per 32 cm².

Embodiment 5 comprises a fetal tissue product of any one of embodiments 1 to 4, wherein the HA is low molecular wight HA.

Embodiment 6 comprises a fetal tissue product of any one of embodiments 1 to 5, wherein about 20% to about 90% of the HA is covalently bonded to a heavy chain 1 of IαI (HC1) complexed with pentraxin 3 (PTX3).

Embodiment 7 comprises a fetal tissue product of any one of embodiments 1 to 5, wherein about 30% to about 75% of the HA is covalently bonded to a heavy chain 1 of IαI (HC1) complexed with pentraxin 3 (PTX3).

Embodiment 8 comprises a fetal tissue product of any one of embodiments 1 to 5, wherein about 20% to about 50% of the HA is covalently bonded to a heavy chain 1 of IαI (HC1) complexed with pentraxin 3 (PTX3).

Embodiment 9 comprises a fetal tissue product of any one of embodiments 1 to 5, wherein about 20% to about 40% of the HA is covalently bonded to a heavy chain 1 of IαI (HC1) complexed with pentraxin 3 (PTX3).

Embodiment 10 comprises a fetal tissue product of any one of embodiments 1 to 9, wherein the fetal tissue comprises amniotic membrane (AM).

Embodiment 11 comprises a fetal tissue product of any one of embodiments 1 to 10, wherein the fetal tissue comprises placental amniotic membrane (AM).

Embodiment 12 comprises a fetal tissue product of any one of embodiments 1 to 11, wherein the fetal tissue product was previously frozen.

Embodiment 13 comprises a fetal tissue product of any one of embodiments 2 to 11, wherein the sheet is circular.

Embodiment 14 comprises a fetal tissue product of any one of embodiments 2 to 11, wherein the sheet has a diameter from 8 millimeters to 16 millimeters.

Embodiment 15 comprises a fetal tissue product of any one of embodiments 2 to 11, wherein the sheet has a diameter from 9 millimeters to 15 millimeters.

Embodiment 16 comprises a fetal tissue product of any one of embodiments 2 to 11, wherein the sheet has a diameter from 10 millimeters to 14 millimeters.

Embodiment 17 comprises a fetal tissue product of any one of embodiments 2 to 11, wherein the sheet has a diameter from 11 millimeters to 13 millimeters.

Embodiment 18 comprises a fetal tissue product of any one of embodiments 2 to 11, wherein the sheet has a diameter of about 12 millimeters.

Embodiment 19 comprises a fetal tissue product of any one of embodiments 1 to 18, wherein the sheet has an average thickness of less than about 200 micrometers.

Embodiment 20 comprises a fetal tissue product of any one of embodiments 1 to 18, wherein the sheet has an average thickness of between about 150 micrometers and about 75 micrometers.

Embodiment 21 comprises a fetal tissue product of any one of embodiments 1 to 20, wherein the sheet has an average thickness of greater than about 50 micrometers.

Embodiment 22 comprises a fetal tissue product of any one of embodiments 1 to 21, wherein the sheet is substantially flat.

Embodiment 23 comprises a fetal tissue product of any one of embodiments 1 to 21, wherein the fetal tissue is not dehydrated.

Embodiment 24 comprises a fetal tissue product of any one of embodiments 1 to 21, wherein water is not removed from the fetal tissue.

Embodiment 25 comprises a fetal tissue product of any one of embodiments 1 to 24, wherein the fetal tissue is terminally sterilized.

Embodiment 26 comprises a fetal tissue product of embodiment 25, wherein the fetal tissue is terminally sterilized by gamma irradiation or electron beam sterilization.

Embodiment 27 comprises a fetal tissue product of any one of embodiments 1 to 26, wherein substantially all of the cells of the fetal tissue are dead.

Embodiment 28 comprises a fetal tissue product of any one of embodiments 1 to 27, wherein the fetal tissue product is terminally sterilized at a temperature at or below 0° C.

Embodiment 29 comprises a fetal tissue product of any one of embodiments 1 to 21, wherein the fetal tissue product is adhered to a backing material.

Embodiment 30 comprises a fetal tissue product of embodiment 29, wherein the backing material is a polyethersulfone (PES) polymer.

Embodiment 31 comprises a fetal tissue product of embodiment 29 or 30, wherein the backing material is gridded.

Embodiment 32 comprises a fetal tissue product of any one of embodiments 1 to 31, wherein the fetal tissue product does not comprise a ring-shaped support structure.

Embodiment 33 comprises a fetal tissue product of any one of embodiments 1 to 32, wherein the fetal tissue product is clear.

Embodiment 34 comprises a fetal tissue product of any one of embodiments 1 to 33, wherein the fetal tissue product is optically clear.

Embodiment 35 comprises a fetal tissue product of any one of embodiments 1 to 34, wherein the fetal tissue product is packaged in normal (0.9%) saline.

Embodiment 36 comprises a fetal tissue product of any one of embodiments 1 to 35, wherein the fetal tissue product is stable at −20° C. to 25° C. for at least one year.

Embodiment 37 comprises a fetal tissue product of any one of embodiments 1 to 36, wherein the fetal tissue product adheres to a surface comprised of lotrafilcon A with a force of greater than 0.0001 pounds per square inch.

Embodiment 38 comprises a fetal tissue product of any one of embodiments 1 to 36, wherein the fetal tissue product adheres to a surface comprised of lotrafilcon A with a force of greater than 0.001 pounds per square inch.

Embodiment 39 comprises a fetal tissue product of any one of embodiments 1 to 36, wherein the fetal tissue product adheres to a surface comprised of lotrafilcon A with a force of greater than 0.01 pounds per square inch.

Embodiment 40 comprises a fetal tissue product of any one of embodiments 1 to 36, wherein the fetal tissue product adheres to a surface comprised of lotrafilcon A with a force of greater than 0.1 pounds per square inch.

Embodiment 41 comprises a fetal tissue product of any one of embodiments 1 to 36, wherein the fetal tissue product adheres to a surface comprised of lotrafilcon A with a force of greater than 1 pound per square inch.

Embodiment 42 comprises a fetal tissue product of any one of embodiments 1 to 36, wherein the fetal tissue product adheres to a surface comprised of lotrafilcon A with a force of greater than 2 pounds per square inch.

Embodiment 43 comprises a fetal tissue product of any one of embodiments 1 to 36, wherein the fetal tissue product adheres to a surface comprised of lotrafilcon A with a force of greater than 5 pounds per square inch.

Embodiment 44 comprises a fetal tissue product of any one of embodiments 1 to 36, wherein the fetal tissue product adheres to a surface comprised of lotrafilcon A with a force of greater than 10 pounds per square inch.

Embodiment 45 comprises a fetal tissue product of any one of embodiments 1 to 36, wherein the fetal tissue product adheres to a surface comprised of lotrafilcon A with a force of greater than 15 pounds per square inch.

Embodiment 46 comprises a fetal tissue product of any one of embodiments 1 to 36, wherein the fetal tissue product adheres to a surface comprised of lotrafilcon A with a force of greater than 20 pounds per square inch.

Embodiment 47 comprises a fetal tissue product of any one of embodiments 1 to 46, wherein the fetal tissue product exhibits increased adhesion to a surface comprised of lotrafilcon A after sterilization with gamma irradiation at temperature of below 0° C.

Embodiment 48 comprises a fetal tissue product of embodiment 47, wherein the fetal tissue product is adhered to the contact lens such that when inserted into an eye of an individual the fetal tissue product contacts the surface of the eye of the individual.

Embodiment 49 comprises a method of treating a disease or disorder of the eye comprising administering the fetal tissue product of any one of embodiments 1 to 48 or the fetal tissue product adhered to a surface of a contact lens to an eye of the individual.

Embodiment 50 comprises a method of embodiment 49, wherein the fetal tissue product is administered to the eye without being adhered to the surface of a contact lens.

Embodiment 51 comprises a method of embodiment 49, wherein the fetal tissue product is administered to the eye adhered to the surface of the contact lens.

Embodiment 52 comprises a method of any one of embodiments 49 to 51, wherein the disease or disorder of the eye comprises dry eye, corneal abrasion, neurotrophic keratitis, corneal epithelial defect, corneal ulcer, or recurrent corneal epithelial erosion.

Embodiment 53 comprises a method of any one of embodiments 49 to 51, wherein the disease or disorder of the eye comprises dry eye disease.

Embodiment 54 comprises a method of any one of embodiments 49 to 51, wherein the disease or disorder of the eye comprises a corneal abrasion.

Embodiment 55 comprises a method of making a treatment for a disease of the eye comprising contacting the fetal tissue product of any one of embodiments 1 to 47 to the surface of a contact lens, thereby adhering the fetal tissue product to the contact lens.

Embodiment 56 comprises a kit for treating a disease or disorder of the eye, the kit comprising the fetal tissue product of any one of embodiments 1 to 47 and any one or more tweezers, a swab, and a surgical tray.

Embodiment 57 comprises a kit of embodiment 56, wherein the fetal tissue is adhered to a backing material.

Embodiment 58 comprises a kit of embodiment 57, wherein the backing material is a polyethersulfone (PES) polymer.

Embodiment 59 comprises a kit of embodiment 57 or 58, wherein the backing material is gridded.

Embodiment 60 comprises a kit of any one of embodiments 57 to 59, wherein the disease or disorder of the eye comprises dry eye disease.

Embodiment 61 comprises a kit of any one of embodiments 57 to 59, wherein the disease or disorder of the eye comprises a corneal abrasion.

Embodiment 62 comprises a kit of any one of embodiments 57 to 59, wherein the disease or disorder of the eye comprises dry eye, corneal abrasion, neurotrophic keratitis, corneal epithelial defect, corneal ulcer, or recurrent corneal epithelial erosion.

### Embodiments 2

Embodiment 1—A fetal tissue product comprising fetal tissue, wherein the fetal tissue product comprises less than about 30 micrograms of hyaluronic acid (HA) per 32 cm².

Embodiment 2—The fetal tissue product of embodiment 1, wherein the fetal tissue or the fetal tissue product is a sheet.

Embodiment 3—The fetal tissue product of embodiment 1 or 2, wherein the fetal tissue product comprises less than about 20 micrograms of hyaluronic acid per 32 cm².

Embodiment 4—The fetal tissue product of embodiment 1 or 2, wherein the fetal tissue product comprises less than about 10 micrograms of hyaluronic acid per 32 cm².

Embodiment 5—The fetal tissue product of any one of embodiments 1 to 4, wherein the HA is low molecular weight HA, optionally wherein the low molecular weight HA comprises an average size of 500 kD or less.

Embodiment 6—The fetal tissue product of any one of embodiments 1 to 5, wherein about 20% to about 90% of the HA is covalently bonded to a heavy chain 1 of IαI (HC1) complexed with pentraxin 3 (PTX3).

Embodiment 7—The fetal tissue product of any one of embodiments 1 to 5, wherein about 30% to about 75% of the HA is covalently bonded to a heavy chain 1 of IαI (HC1) complexed with pentraxin 3 (PTX3).

Embodiment 8—The fetal tissue product of any one of embodiments 1 to 5, wherein about 20% to about 50% of the HA is covalently bonded to a heavy chain 1 of IαI (HC1) complexed with pentraxin 3 (PTX3).

Embodiment 9—The fetal tissue product of any one of embodiments 1 to 5, wherein about 20% to about 40% of the HA is covalently bonded to a heavy chain 1 of IαI (HC1) complexed with pentraxin 3 (PTX3).

Embodiment 10—The fetal tissue product of any one of embodiments 1 to 9, comprising a total HA content of about 0.2 μg or less of HA.

Embodiment 11—The fetal tissue product of any one of embodiments 1 to 9, comprising a total HA content of 0.16 μg or less.

Embodiment 12—The fetal tissue product of any one of embodiments 1 to 11, wherein the fetal tissue comprises amniotic membrane (AM), optionally wherein the amniotic membrane is placental amniotic membrane.

Embodiment 13—The fetal tissue product of any one of embodiments 1 to 12, wherein the fetal tissue does not comprise umbilical cord.

Embodiment 14—The fetal tissue product of any one of embodiments 1 to 13, wherein the fetal tissue was previously frozen.

Embodiment 15—The fetal tissue product of any one of embodiments 2 to 13 wherein the sheet is circular.

Embodiment 16—The fetal tissue product of any one of embodiments 2 to 15, wherein the sheet has a diameter from 8 millimeters to 16 millimeters.

Embodiment 17—The fetal tissue product of any one of embodiments 2 to 15, wherein the sheet has a diameter of about 12 millimeters.

Embodiment 18—The fetal tissue product of any one of embodiments 2 to 17, wherein the sheet has an average thickness of less than about 200 micrometers.

Embodiment 19—The fetal tissue product of any one of embodiments 2 to 17, wherein the sheet has an average thickness of between about 200 micrometers and about 1 micrometer.

Embodiment 20—The fetal tissue product of any one of embodiments 2 to 17, wherein the sheet has an average thickness of between about 30 micrometers and about 10 micrometers.

Embodiment 21—The fetal tissue product of any one of embodiments 2 to 17, wherein the sheet has an average thickness of between about 25 micrometers and about 15 micrometers.

Embodiment 22—The fetal tissue product of any one of embodiments 2 to 17, wherein the sheet has an average thickness of greater than about 5 micrometers.

Embodiment 23—The fetal tissue product of any one of embodiments 2 to 22, wherein the sheet is substantially flat.

Embodiment 24—The fetal tissue product of any one of embodiments 1 to 23, wherein the fetal tissue is not dehydrated.

Embodiment 25—The fetal tissue product of any one of embodiments 1 to 24, wherein water is not substantially removed from the fetal tissue.

Embodiment 26—The fetal tissue product of any one of embodiments 1 to 25, wherein substantially all of the cells of the fetal tissue are dead.

Embodiment 27—The fetal tissue product of any one of embodiments 1 to 26, wherein the fetal tissue product is terminally sterilized at a temperature at or below 0° C.

Embodiment 28—The fetal tissue product of any one of embodiments 1 to 27, wherein the fetal tissue product is adhered to a backing material.

Embodiment 29—The fetal tissue product of embodiment 28, wherein the backing material is a polyethersulfone (PES) polymer.

Embodiment 30—The fetal tissue product of embodiment 28 or 29, wherein the backing material is gridded.

Embodiment 31—The fetal tissue product of any one of embodiments 1 to 30, wherein the fetal tissue product does not comprise a ring-shaped support structure.

Embodiment 32—The fetal tissue product of any one of embodiments 1 to 31, wherein the fetal tissue product is clear.

Embodiment 33—The fetal tissue product of any one of embodiments 1 to 32, wherein the fetal tissue product is optically clear.

Embodiment 34—The fetal tissue product of any one of embodiments 1 to 33, wherein the fetal tissue product is packaged in normal (0.9%) saline.

Embodiment 35—The fetal tissue product of any one of embodiments 1 to 34, wherein the fetal tissue product is stable at 20° C. to 25° C. for at least one year.

Embodiment 36—The fetal tissue product of any one of embodiments 1 to 35, wherein the fetal tissue product adheres to a substrate comprised of lotrafilcon A with a force of greater than 0.0001 pounds per square inch.

Embodiment 37—The fetal tissue product of any one of embodiments 1 to 35, wherein the fetal tissue product adheres to a substrate comprised of lotrafilcon A with a force of greater than 0.01 pounds per square inch.

Embodiment 38—The fetal tissue product of any one of embodiments 1 to 35, wherein the fetal tissue product adheres to a substrate comprised of lotrafilcon A with a force of greater than 1, 2, 5, 10, 15, or 20 pounds per square inch.

Embodiment 39—The fetal tissue product of any one of embodiments 1 to 38, wherein the fetal tissue product exhibits increased adhesion to a surface comprised of lotrafilcon A after sterilization with gamma irradiation at a temperature below 0° C.

Embodiment 40—The fetal tissue product of any one of embodiments 1 to 39, wherein the fetal tissue product is adhered to a contact lens or collagen shield such that when inserted into an eye of an individual the fetal tissue product contacts the surface of the eye of the individual.

Embodiment 41—The fetal tissue product of any one of embodiments 1 to 38, wherein the fetal tissue product exhibits increased adhesiveness to a surface of an eye or a substrate.

Embodiment 42—The fetal tissue product of embodiment 41, wherein the substrate comprises a soft contact lens.

Embodiment 43—The fetal tissue product of embodiment 41 or 42, wherein the substrate comprises a collagen corneal shield, or collagen shield.

Embodiment 44—The fetal tissue product of any one of embodiments 41 to 43, wherein the substrate comprises a bandage contact lens.

Embodiment 45—An ophthalmic device comprising the fetal tissue product of any one of embodiments 1 to 44 adhered or contacted to a substrate.

Embodiment 46—The ophthalmic device of embodiment 45, wherein the fetal tissue product is not cross-linked to the substrate.

Embodiment 47—The ophthalmic device of embodiment 45 or 46, wherein the substrate comprises a collagen shield or a collagen corneal shield.

Embodiment 48—The ophthalmic device of any one of embodiments 45 to 47, wherein the substrate comprises a bandage contact lens.

Embodiment 49—The ophthalmic device of any one of embodiments 45 to 47, wherein the substrate comprises a soft contact lens.

Embodiment 50—The ophthalmic device of embodiment 49, wherein the soft contact lens comprises Senofilcon A, balafilcon A, methafilcon A, comfilcon A, nelfilcon A hydrogel, or silicon hydrogel.

Embodiment 51—The ophthalmic device of any one of embodiments 45 to 50, wherein the substrate comprises a circular structure, square structure, rectangular structure or an irregular structure.

Embodiment 52—The ophthalmic device of any one of embodiments 45 to 50, wherein the substrate is circular.

Embodiment 53—The ophthalmic device of any one of embodiments 42 to 52, wherein the substrate is configured to be placed on a surface of an eye.

Embodiment 54—A method of treating a disease or disorder of an eye of an individual in need thereof comprising administering the fetal tissue product of any one of embodiments 1 to 44, or the ophthalmic device of any one of embodiments 45 to 53, to the eye of the individual.

Embodiment 55—The method of embodiment 54, wherein the fetal tissue product or ophthalmic device is administered to a surface of the eye of the individual.

Embodiment 56—The method of embodiment 54, wherein the fetal tissue product or ophthalmic device is administered via the surface of the contact lens, wherein the tissue product is adhered to the contact lens and placed on the surface of the eye of the individual, wherein the fetal tissue product contacts the eye.

Embodiment 57—The method of embodiment 54, wherein the fetal tissue product or ophthalmic device is administered via collagen shield, wherein the tissue product is adhered to the collagen shield and placed on the surface of the eye of the individual, wherein the fetal tissue product contacts the eye.

Embodiment 58—The method of embodiment 54, wherein the fetal tissue product is administered to an intravitreal, epiretinal, or subretinal space of the eye, optionally during glaucoma or oculoplastic surgeries the eye.

Embodiment 59—The method of embodiment 54, wherein the fetal tissue product is administered via a lacrimal intubation tube.

Embodiment 60—The method of embodiment 54, wherein the disease or disorder of an eye is a disease or disorder of the surface of the eye.

Embodiment 61—The method of any one of embodiments 54 to 60, wherein the disease or disorder of the eye comprises dry eye disease, corneal abrasion, superficial punctate keratitis, non-healing injury/wound, post debridement, epithelial basement membrane dystrophy, neurotrophic keratitis, corneal epithelial defect, corneal ulcer, or recurrent corneal epithelial erosion.

Embodiment 62—The method of any one of embodiments 54 to 60, wherein the disease or disorder of the eye comprises dry-eye-disease.

Embodiment 63—The method of any one of embodiments 54 to 60, wherein the disease or disorder of the eye comprises a corneal abrasion.

Embodiment 64—The method of any one of embodiments 54 to 60, wherein the disease or disorder of the eye comprises neurotrophic keratitis.

Embodiment 65—The method of any one of embodiments 54 to 60, wherein the disease or disorder of the eye comprises superficial punctate keratitis.

Embodiment 66—The method of any one of embodiments 54 to 60, wherein the disease or disorder of the eye comprises Stevens-Johnson syndrome, glaucoma, macular hole, retinal detachment, macular degeneration or oculoplastic diseases.

Embodiment 67—The method of any one of embodiments 54 to 60, wherein the disease or disorder of the eye comprises a burn, optionally wherein the burn is a chemical burn or a thermal burn.

Embodiment 68—The method of any one of embodiments 54 to 60, wherein the disease or disorder of the eye comprises recovery from an eye surgery.

Embodiment 69—The method of any one of embodiments 54 to 68, wherein the method results in reduced incidence of one or more adverse events to the individual.

Embodiment 70—The method of embodiment 69, wherein the reduced one or more adverse events comprise an ocular ulcer.

Embodiment 71—The method of embodiment 69 or 70, wherein the reduced one or more adverse events comprise corneal infiltrates.

Embodiment 72—The method of any one of embodiment 54 to 71, further comprising administering one or more antibiotics.

Embodiment 73—The method of any one of embodiment 54 to 71, further comprising administering artificial tears.

Embodiment 74—A method of making a fetal tissue product comprising obtaining a fetal tissue, subjecting it to terminal sterilization, isolating and recovering the fetal tissue to isolate the fetal tissue product of any one of embodiments 1 to 44.

Embodiment 75—The method of embodiment 74, wherein the fetal tissue undergoes cryopreservation and terminal sterilization.

Embodiment 76—The method of embodiment 74 or 75, wherein the terminal sterilization comprises gamma irradiation or electron beam sterilization.

Embodiment 77—The method of embodiment 74, wherein the terminal sterilization comprises gamma irradiation.

Embodiment 78—The method of embodiment 74, wherein the terminal sterilization comprises electron beam irradiation.

Embodiment 79—A kit for treating a disease or disorder of an eye, the kit comprising the fetal tissue product of any one of embodiments 1 to 44 and one or more substrates, and, optionally, any one or more of saline or buffer, tweezers, swabs, forceps, or surgical trays.

Embodiment 80—The kit of embodiment 79, wherein the fetal tissue product is adhered to a backing material.

Embodiment 81—The kit of embodiment 80, wherein the backing material is a polyethersulfone (PES) polymer.

Embodiment 82—The kit of embodiment 80 or 81, wherein the backing material is gridded.

Embodiment 83—The kit of any one of embodiments 79 to 82, wherein the fetal tissue product is stored in an aqueous solution.

Embodiment 84—The kit of embodiment 83, wherein the aqueous solution is a 0.9% saline solution.

Embodiment 85—The kit of any one of embodiments 79 to 84, wherein the disease or disorder of the eye comprises dry-eye-disease.

Embodiment 86—The kit of any one of embodiments 79 to 84, wherein the disease or disorder of the eye comprises a corneal abrasion.

Embodiment 87—The kit of any one of embodiments 79 to 84, wherein the disease or disorder of the eye comprises dry eye disease, corneal abrasion, superficial punctate keratitis, non-healing injury/wound, post debridement, epithelial basement membrane dystrophy, neurotrophic keratitis, corneal epithelial defect, corneal ulcer, or recurrent corneal epithelial erosion.

Embodiment 88—The kit of any one of embodiments 79 to 84, wherein the disease or disorder of the eye comprises a dry-eye-disease.

Embodiment 89—The kit of any one of embodiments 79 to 84, wherein the disease or disorder of the eye comprises a superficial punctate keratitis.

Embodiment 90—The kit of any one of embodiments 79 to 84, wherein the disease or disorder of the eye comprises a neurotrophic keratitis.

Embodiment 91—The kit of any one of embodiments 79 to 84, wherein the disease or disorder of the eye comprises Stevens-Johnson syndrome, glaucoma, macular hole, retinal detachment, macular degeneration or oculoplastic diseases.

Embodiment 92—The kit of any one of embodiments 79 to 84, wherein the disease or disorder of the eye comprises a burn, optionally wherein the burn is a chemical burn or a thermal burn.

Embodiment 93—The kit of any one of embodiments 79 to 84, wherein the disease or disorder of the eye comprises recovery from an eye surgery.

Embodiment 94—The kit of any one of embodiments 79 to 93, wherein the one or more substrates comprise a contact lens comprising a bandage contact lens (BCL), a collagen shield (CS) or a collagen corneal shield.

Embodiment 95—The kit of embodiments 79 to 93, wherein the contact lens comprises a soft contact lens comprising Senofilcon A, balafilcon A, comfilcon A, methafilcon A, comfilcon A, nelfilcon A hydrogel or silicon hydrogel.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Processing AM Product and Usage (Sterilization, Storage, Unpackaging, and Preparation of the AM Product)

The fetal tissue product disclosed is also known as amniotic membrane (AM) product. The AM product was generated using cryopreservation method A (protocol described e.g., in method of manufacturing and in Example 2 below) and processed using a SteriTek® preservation process, and/or a CryoTek® technology (technology of BioTissue® and BioTissue Holdings Inc.). The SteriTek® preservation processing of an amniotic membrane (AM) tissue preservation yields a shelf-stable product while maintaining a natural hydration of a tissue product. The CryoTek® cryopreservation process maintains the structural and biological integrity of a natural tissue. The fetal tissue or AM tissue disclosed is processed by CryoTek® cryopreservation technology and is then terminally sterilized e.g., under gamma irradiation to yield a fully hydrated AM product that is stable at room temperature e.g., under a controlled room temperature. The AM product is manufactured, using the SteriTek® preservation process, from donated placenta or birth tissue according to Current Good Tissue Practice (CGTP) and Current Good Manufacturing Practice (CGMP) regulations established by the U.S. Food and Drug Administration (FDA). Specifically, the AM product is terminally sterilized in hydrated state (not dehydrated) under icy conditions below 0° C. The AM product is cryopreserved and is aseptically processed, cell devitalized and terminally sterilized via gamma irradiation with a Sterility Assurance Level (SAL) of 10-6 (ISO 11137; VDmax25 Gamma Radiation Validation for Amniotic Membrane (AM) and Umbilical Cord (UC) Graft Family Report #797150758-F dated Aug. 12, 2015). The AM product is stored in a pouch packaging. The inner and outer pouch packaging as well as the PES backing are sterilized via gamma irradiation according to ISO 11137 prior to use. Therefore, the final AM product is not re-sterilized or autoclaved before use. The AM product in the pouch is stored in saline (200 μl of 0.9% w/v sodium chloride (NaCl) solution) and is delivered on a non-implantable, gridded backing (Polyethersulfone backing, also known as PES backing) for easier handling and application; the gridded backing with a slit in the middle aids in the detachment of the AM product. The 200 μl saline storage solution is to prevent the AM tissue product drying out during storage.

The fetal tissue product or AM product disclosed is a sheet product that is not heat-treated, is not dehydrated, and once unpacked does not require rehydration. As indicated, the AM product is stored in 200 μl saline storage solution and the HC-HA/PTX3 content of the product is preserved. The AM product is sealed in a pouch that has an inner and outer pouch. The AM product produced by the methods disclosed herein retains the water content so that it is not a dehydrated product but rather stays hydrated. The fetal tissue product/AM product disclosed is processed to a sterile product using cryopreservation method A. The manufacturing and sterilization procedures disclosed produce an AM product having strong adhesive properties that enable it to adhere onto surfaces e.g., contact lens surfaces immediately without any prompting. The strong adhesive properties present on the AM product is seen on both the epithelial and stroma sides of the AM product. Biochemical Analysis data available related to the AM product and AmnioGraft™ shows both products retain HC-HA/PTX3 that is innate to fresh amniotic tissue. The AM product also has less hyaluronic acid (ug HA per cm2 AM tissue) compared to other amniotic products. Without being bound by theory this reduced HA content contributes to the strong adhesive properties of the AM product. Nonetheless, the AM product retains HA that is bound in the HC-HA/PTX3 complex. However, the HA amount of the AM product is less and of smaller molecular weight (see agarose gel electrophoresis data). The AM product is not a dehydrated product, it is hydrated. Because it adheres on surfaces without need for suturing or gluing in place, the AM product can be placed directly on the surface of the eye, e.g., the corneal surface without any tools/vehicles and can be used to treat ocular conditions following adherence. Unpacking or following instructions in the shipment (FIG. 5A-5F; FIG. 10A-FIG. 10B).

Once on the ocular surface, the AM product exerts anti-inflammatory, anti-scarring and anti-angiogenic actions to promote wound healing, and supports epithelial adhesion and differentiation. The AM product disclosed has numerous applications as outlined herein. Briefly, the AM product targeted to treat eye disorders or diseases, e.g., corneal diseases. Examples of such disorders include dry eye disease (DED), neurotrophic keratitis, corneal epithelial defect, corneal ulcer, recurrent corneal epithelial erosion, Superficial keratitis, punctate epithelial keratitis, keratitis, punctate epithelial erosion, epithelial basement membrane dystrophy, etcetera.

### Example 2: HA Assay Conducted on Amniotic Membrane (AM) Product Extracts

Placenta: Donors of the birth tissue are tested and found non-reactive for HIV-1 & HIV-2 Antibody, HIV-1 (RNA-NAT), Hepatitis B Surface Antigen (HBsAg)Hepatitis B Core Antibody (HBcAb), Hepatitis B Virus (HBV, DNA-NAT), Hepatitis C Antibody (HCVAb), Hepatitis C Virus (HCV, RNA-NAT), Syphilis (RPR), HTLV I & II Antibody (HTLV I/II Ab), West Nile Virus (WNV, RNA-NAT).

Amniotic membrane (AM) sheet products tested: The fetal tissue product refers to the amniotic membrane (AM) product described herein. One example of an AM product of the present disclosure is a CAM360® AmnioGraft™ product, which is also referred to as CAM360 AmnioGraft, or CAM360® AG™, or CAM360 AG (BioTissue Holdings Inc, Miami, FL). An AM product disclosed is an amniotic membrane tissue product. An AM product described herein is not heated. An AM product described herein is not dehydrated. An AM product described herein is processed using the methods disclosed herein to produce a hydrated product, which retains the biological properties of an AM tissue and purified as disclosed herein. An AM product can be processed by the methods described alone or in combination with any other methods of preparation or processing, etcetera. A fetal tissue product or an AM product described herein may be used alone as a stand-alone product, e.g., a therapeutic or it can be used in combination with any other suitable product(s) or therapeutic(s).

Ambio2® and AmbioDisk® were used in the testing (Corza Medical, Westwood, MA manufactured by StimLabs, LLC) are amniotic membrane sheet products or AM grafts for ophthalmic use. Ambio2® and AmbioDisk® are Clearify™ processed, dehydrated, AM allografts used across a variety of surgical applications. Clearify™ process retains amniotic intermediate layer, a membrane barrier found between the amnion and chorion layers used for surgical regeneration of ocular surface tissues and cells.

XcellerEYES™ (Oculus Biologics, Willowbrook, IL, manufactured by Elutia) is a lyophilized amniotic membrane allograft dehydrated amniotic membrane that is opaque and used for the ocular surface with no rehydration recommended.

Cryopreservation Method A: AM product samples used for characterization may be prepared by a cryopreservation method A as described herein, the method used alone, or in combination with any other method(s). In some embodiments, a cryopreservation method A comprises processing of an AM tissue aseptically performed under Class II Biological Safety Cabinet using minimal manipulation of an AM tissue. Briefly, frozen placenta was thawed under controlled conditions and the amniotic membrane (AM) was isolated from the placenta. The AM was further separated from the chorion. The AM was then subjected to a series of steps: 1) Wash with saline; 2) Gently remove blood from stromal side of AM; 3) Wash with saline (Soak+Gentle Massage Optional (if blood still present)); 4) Repeat wash until tissue is cleaned to its entirety; 5) Wash with saline, Swirl and let sit—1 min; 6) Wash saline Swirl and let sit—1 min; 7) Wash with saline—Let Sit; 8) Wash with saline. AM was then placed on sterile membrane, e.g., a polyethersulfone (PES) membrane, with stromal side down and the tissue graft was cut according to size. The tissue grafts were then placed in pouches filled with storage solution (e.g., a saline or an isotonic solution) and sealed. The products were kept in sub-zero freezing temperature e.g., in a freezer (−20° C.) until needed e.g., to process and prepare the fetal tissue product or the AM product. A gamma irradiation sterilization process for medical devices was utilized which applied to i) a sterilization dose of 25 kGy, b) the establishment of a radiation sterilization dose which is primarily set on bioburden (i.e., initial contamination that described population of active pathogens before sterilization). The optimal radiation dosage 25 kGy is routinely used in many tissue banks.

The AM product described herein comprises an example of a cryopreserved AM that undergoes terminal sterilization to enable room temperature storage as a sterile product with sterility assurance level of 10⁻⁶. The AM product described herein generated by a cryopreservation method A is not a dehydrated product. The AM product described herein comprises a hydrated product.

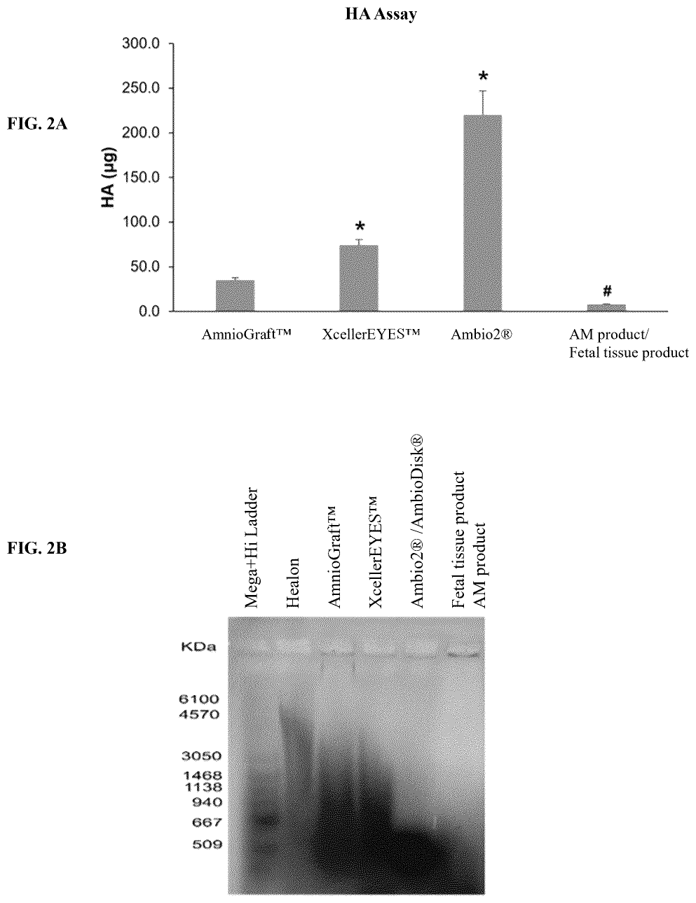

Characterizing AM sheet products: A quantitative hyaluronic acid (HA) Assay was conducted on AM extracts (AME) derived from (1) Ambio2®/AmbioDisk®, (2) AmoGraft™ (3) XcellerEYES™ and (4) the fetal tissue product or AM product described herein. Briefly, four different placental tissue products isolated from amniotic membrane were extracted and processed as shown in Table 1. The HA content or concentration of each AME was measured by the HA Assay Kit (Corgenix, Cat #029-001RU, Broomfield, CO). To directly quantify the content of HA within the AM sheet product, 5 μl of the AME from each AM sheet product was analyzed. The results showed that the total HA content or amounts of HA in XcellerEYES™ and Ambio2® (AmbioDiskK®) were significantly higher than those of AmmioGraft™ (p<0.05). The results also showed that the total amount of HA in the fetal tissue product or AM product disclosed was significantly lower than that of XcellerEYES™, Ambio2®, and AmoGraft™ (p<0.05, FIG. 2A). The HA amount in the solution of the fetal tissue product/AM product was 0.16 μg, which didn't affect the amount of HA in the fetal tissue product or AM product overall (Table 2). The HA content per 32 cm² was 35.0 μg for AmmoGraft™, 74.4 mg for XcellerEYES™, 220.5 μg for AMBIO2®, and 8.1 μg for the fetal tissue product/AM product disclosed herein. Thus, the study confirmed the fetal tissue product/AM product disclosed had the lowest total HA content of any product.

TABLE 1
Preparation of AM extract for characterization

| | Wet Tissue | | Dry Tissue |
|---|---|---|---|
| | (AmnioGraft ™ and AM product | | (XcellerEYES ™ and |
| Steps | disclosed) | | Ambio2 ® |
| Tissue size | 3.5 × 3.5 | cm | Same size |
| Storage | −80° | C. | Room temperature |
| | | | (Ambio2 ®, Apollo, |
| | | | AmnioFix, etc) |

| | | |
|---|---|---|
| Thaw | thaw in 4° C. refrigerator for 15-20 | — |
| | min | |
| Preparation | Put tissue in 10 ml of chilled PBS for | Same to wet tissue |
| | 1 min and take out | |
| Dry | Dry the tissue on a sterile absorbent | same |
| | towel 3 secs for each side | |
| Pulverization | FreezerMill (Small vial) per WI-0198 | same |
| Transfer | Transfer the tissue to a tube | same |
| Extraction | Add 0.05 ml of 1x PBS (per 3.5 cm × | Same |
| | 3.5 cm AM tissue) to the tube above | |
| | at 1:1 (g/ml) and mix. | |
| | Rotate for 1 h at 30 rpm using the | |
| | Tube Rotator placed in the 4° C. | |
| | refrigerator. | |
| Centrifugation | 21,130 g (maximum speed) at 4° C. | same |
| | for 15 min | |
| AME | Collect supernatant (AME) | same |
| Western Blot | Take out the lyophilized sample and | Add the same amount of |
| | reconstitute the sample according to | buffer to the lyophilized dry |
| | the results of HA assay. If no | tissue sample as wet tissue. If |
| | lyophilization, skip this step. | no lyophilization, skip this |
| | Then, load the sample containing 5 μg | step. |
| | of HA in each well. | Then, load the sample in |
| | | each well with the same |
| | | volume with wet tissue. |
| NO Assay | Reconstitute the lyophilized sample | Add the same amount of |
| | according to the results of HA assay. | medium to the lyophilized |
| | If no lyophilization, skip this step. | dry tissue sample as wet |
| | Then, load the sample in each well | tissue. If no lyophilization, |
| | and make sure the final concentration | skip this step. |
| | of HA is 20 μg/ml. | Then, load the sample in |
| | | each well with the same |
| | | volume with wet tissue. |

Agarose Gel Electrophoresis: To further qualify whether changes in total HA contents might affect the MW of HA, agarose gel electrophoresis was used to compare AME from the 4 products, XcellerEYES™, Ambio2@/AmbioDisk®), AmnioGraft® and the fetal tissue product or AM product described herein. Agarose gel electrophoresis was performed to analyze the HA size in the AME of the four different AM sheet products. The molecular weight of hyaluronic acid (HA) in internal reference material (IRM) or amniotic membrane extract (AME) was loaded at the same amount about 5 or 10 μg of HA per lane on a 0.5% agarose gel and run at 70 V for 2.5 hour. The gel was stained with 0.005% Stains-all dye in 50% ethanol overnight (˜16 h) at 25° C. while covered in foil. Next, the gel was de-stained for six hours in water while exposed to light (no coverings) and the molecular weight of HA in the samples (smear on the agarose gel) was estimated by comparison to the high molecular weight (HMW) standard shown here as the HA Hi-ladder and Healon (˜5000 kDa) as the high molecular weight hyaluronic acid (HA) protein (HMW HA) control (FIG. 2B).

Results: AmnioGraft™ and XcellerEYES™ showed a faint high molecular smearing staining pattern. In contrast, Ambio2® and the fetal tissue product/AM product of the present disclosure did not show any high molecular smearing and Ambio2® had a higher MW HA protein than the HA protein detected in the fetal tissue product or AM product disclosed herein (FIG. 2B).

Compared to the positive HA control, that is, Healon, of which the MW is known in the range of 1000 to 6000 kDa, the MW of HA in both AmnioGraft and XcellerEYES™ was significantly higher than the MW of HA in AmbioDisk® and the fetal tissue product/AM product disclosed herein. For example, the MW of HA for the fetal tissue product/Am product disclosed was lower than 495 kDa (FIG. 2B). These data indicate that terminal sterilization can degrade HA into a smaller MW. Small MW HA is a feature of the fetal tissue product/AM product disclosed. Without being bound by theory, the significant reduction of total HA content (FIG. 2A) and notable reduction of the MW of HA (FIG. 2B) in the fetal tissue product/AM product disclosed explains why it attaches well to a collagen shield (CS) or bandage contact lens (BCL) for in-office application. The fetal tissue product/AM product disclosed attaches well to CS or BCL because of an increased adhesive property caused by the significant reduction of total HA in the product which also facilitates lubrication.

Amniotic Membrane Extract products: Four AME products were assessed in various experiments disclosed. The AME products include (1) XcellerEYES™, (2) Ambio2®/AmbioDisk®, (3) AmnioGraft™ which was used as a control in some experiments described, and (4) the fetal tissue product or AM product disclosed. The AME products display different biological characteristics. For example, the XcellerEYES™ product is an AM graft that undergoes dehydration by lyophilization, which removes water through sublimation without transition through a liquid phase. XcellerEYES™ is stored at room temperature although it is not terminally sterilized. Further, as a comparison both AmbioDisk® and XcellerEYES™ are dry sheet products. Conversely, the fetal tissue product/AM product disclosed is not dehydrated but is an example of a hydrated, cryopreserved AM that undergoes terminal sterilization to enable room temperature storage as a sterile product with sterility assurance level of 10⁻⁶. In contrast to AmnioGraft™, which is commonly used for ocular surface reconstruction, the fetal tissue product or AM product is shelf-stable that facilitates in-office application under a contact lens e.g., a collagen shield or bandage contact lens for the treatment of ocular surface diseases including neurotrophic keratitis, superficial punctate keratopathy, and dry eye disease. Table 2 summarizes the quantity of hyaluronic acid (HA amount) in exemplary commercially available amniotic membrane products Table 3 summarizes the processing methods of the exemplary commercially available amniotic membrane (sheet) products. Differences in the processing or manufacturing methods, storage, transportation, and/or sterility review protocols used, these exemplary AM sheet products likely vary in biological characteristics. Nonetheless, there has been a lack of understanding or clarity on the effect of certain steps of tissue manufacturing, such as for example, dehydration or terminal sterilization of a tissue and whether these processes alter the natural biological properties of the AM tissue. Further studies below were conducted to characterize the amniotic membrane (AM) HC-HA/PTX3 complex disclosed herein.

TABLE 2
HA Amount in different products and associated P Value

| | | | | Fetal tissue | Fetal tissue |
|---|---|---|---|---|---|
| | | | | product (AM | product (AM |
| Product | AmnioGraft ™ | XcellerEYES ™ | Ambio2 ® | product) | solution) |
| Total HA (μg) | 35.0 | 74.4 | 220.5 | 8.1 | 0.16 |
| P Value | | 0.0002 | 0.0038 | 0.0003 | |

HA amount in the fetal tissue product solution or AM product solution = 0.375 μg/ml (HA concentration) × 0.015 ml (volume per pouch) × 28.32 (number of pouches) = 0.16 μg. The number of pouches = 32 cm² (total area)/1.13 cm²(area per pouch) = 28.32 pouces FSTP - fetal tissue product or amniotic membrane (AM) product disclosed herein.

TABLE 3
Composition of Processing Methods of Representation Products

| | | | Terminal | |
|---|---|---|---|---|
| | Aseptic | Preservation | steril- | |
| product | processing | method | ization | Storage |
| AmnioGraft ™ | + | Cryopreservation | − | Hydrated |
| Fetal tissue | + | Cryopreservation | + | Hydrated |
| product/AM | | | | |
| Product of this | | | | |
| disclosure | | | | |
| AmbioDisk ®/ | + | Heat dehydration | + | Dry |
| Ambio2 ® | | | | |
| XcellerEYES ™ | + | Lyophilization | − | Dry |

### Example 3: Characterization of Amniotic Membrane (AM) HC-HA/PTX3 Complex

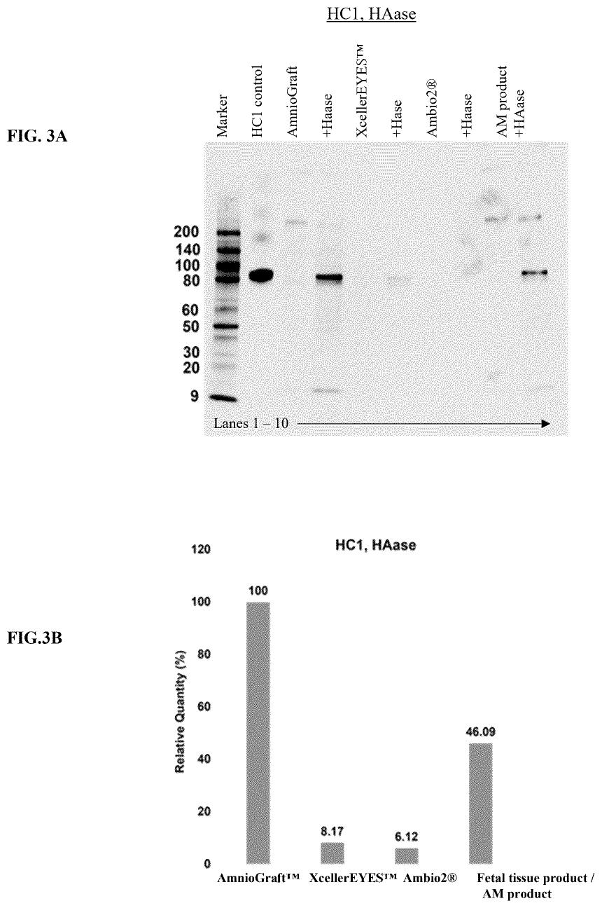

Western blot (WB) for HC1 analysis was conducted to assess preservation of biological characteristics e.g., preservation of HC-HA/PTX3 macromolecules (other than HA) e.g., preservation of heavy chain 1 (HC1), and pentraxin 3 (PTX3). Densitometry of western blots as described above was performed to determine the presence of HC-HA/PTX3. AME of the aforementioned four products, AmnioGraft™, XcellerEYES™, Ambio2® (Ambio2® is also known as Ambio2®), and the fetal tissue product/AM product disclosed. The AME was treated with or without hyaluronidase (Hase) digestion to release covalently bound heavy chain 1 (HC1), was loaded at 2 μg HA per lane followed by immunoblot analysis using an anti-HC1 antibody. Compared to the positive control, i.e., human recombinant HC1, the high molecular weight HMW band on the top of the gel was cleaved by HAase digestion to yield an increase in the intensity of the −80 kDa HC1 fragment. Compared to that of human recombinant HC1 control loaded at 20 mg (FIG. 3A, Lane 2), the band density of HC1 released by hyaluronidase was the highest in AmnioGraft (67% of the HC1 control), followed by the fetal tissue product/AM product (31%) disclosed and much less in the XcellerEYES™ (5%), and Ambio2® (4%) products. These data indicate the HC-HA/PTX3 complex was effectively preserved by the cryopreservation method A. Cryopreservation method A. (FIG. 3A). In contrast, a faint HC1 band was detected in XcellerEYES™ XCELLEREYES™ but none was found in Ambio2®AMBIO2® (FIG. 3A). Densitometry estimated the relative HC1 amount to be 8.17%, 6.12% and 46.09% in XcellerEYES™, Ambio2® and the fetal tissue product or AM product disclosed, respectively, when compared to that of AmnioGraft™ (FIG. 3B).

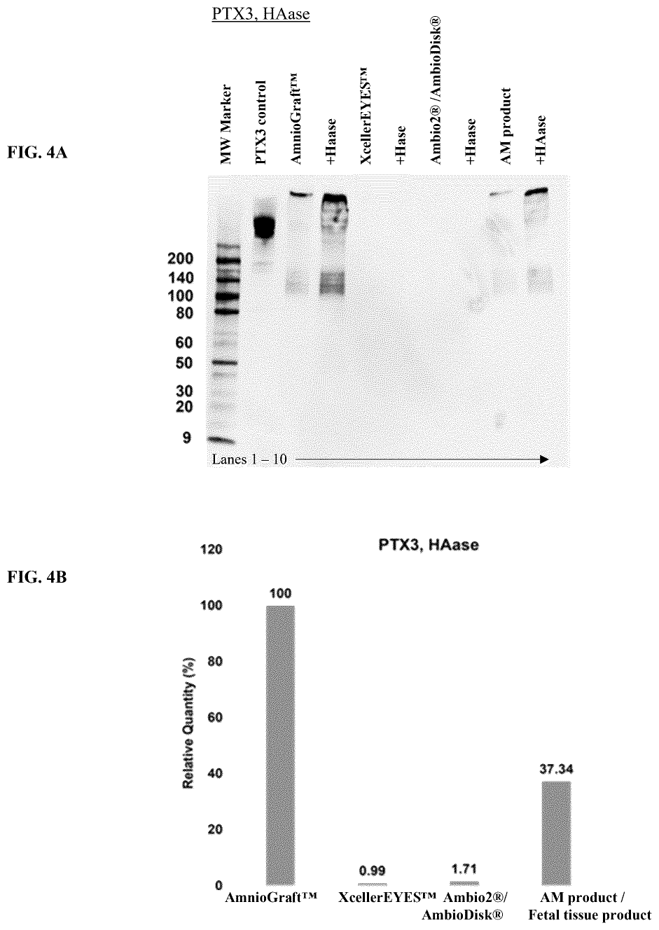

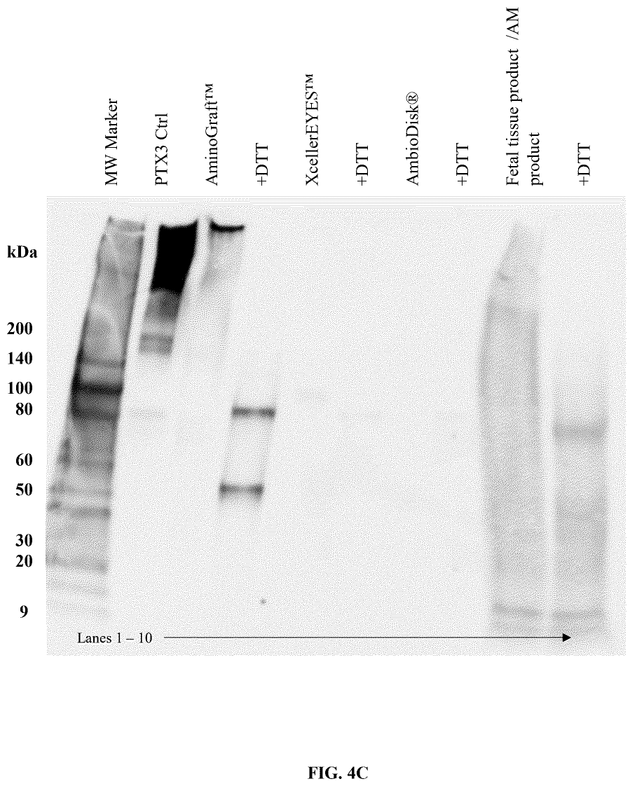

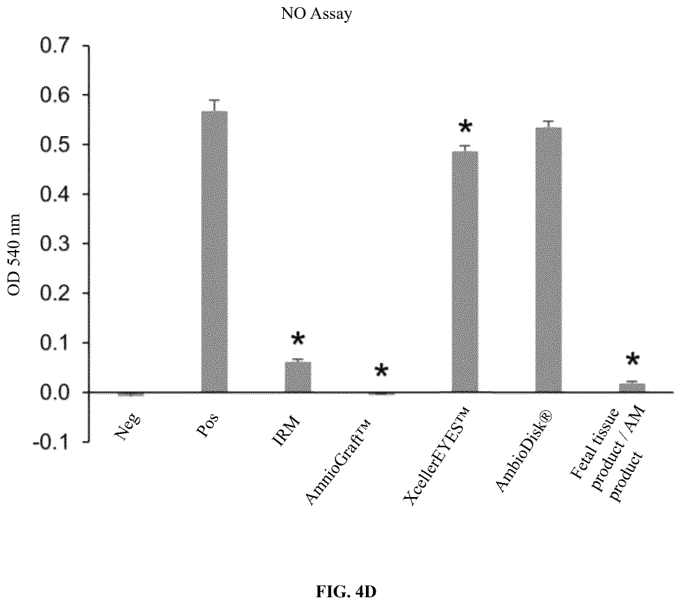

Western blot analysis for PTX3: Similar results to the HC1 findings were obtained for the PTX3 western blot (FIG. 4A) and hyaluronidase digestion and PTX3 (FIG. 4B) with additional DTT reduction (FIG. 4C). Briefly, AME from each product was added at 25 mg/mL of HA in comparison to 20 mg/mL of IRM (*P<0.05 for AmnioGraft™ and the fetal tissue product/AM product compared to positive control and *P<0.0001 for XcellerEYES™ compared to AmnioGraft™ or the fetal tissue product/AM product disclosed. AME of the aforementioned four products, AmnioGraft™, XcellerEYES™, Ambio2® (or AmbioDisk®), and the fetal tissue product/AM product disclosed herein, with or without hyaluronidase (Hase) digestion to release pentraxin 3 (PTX3) which is complexed with heavy chain 1—hyaluronic acid (HC-HA), was loaded at 2 μg HA per lane followed by immunoblot analysis using an anti-PTX3 antibody. The results showed that compared to the PTX3 positive control, which is an octamer, high molecular weight (HMW) PTX3 smear was detected in AmnioGraft™ and the fetal tissue product/AM product of the present disclosure only after HAase digestion, confirming the presence of HC-HA/PTX3 complex. In contrast, no such PTX3 smear was detected in the lanes loaded with extracts from XcellerEYES™ and Ambio2®/AmbioDisk® (FIG. 4A). Densitometry estimated the relative complexed PTX3 amount to be 0.99%, 1.71% and 37.34% in XcellerEYES™, Ambio2®/AmbioDisk® and the fetal tissue product/AM product, respectively, compared to that of AmnioGraft™. This finding suggested that there was more HC-HA/PTX3 in AmnioGraft™ and the AM product disclosed than XcellerEYES™ or Ambio2®. The relative quantities of these measures are shown in the bar graph in FIG. 4B. These findings are consistent with the results noted in HC1 Western blot assay shown in FIG. 3A and FIG. 3B. To circumvent the challenge of quantifying a smear, densitometry of the PTX3 dimer seen in the PTX3 blot after hyaluronidase digestion that was followed by reduction by DTT, detected the highest band in AmnioGraft™ (84% of the PTX3 control) followed by the fetal tissue product/AM product disclosed, however, this band was not detectable in XcellerEYES™ and AmbioDisk® (FIG. 4C). These results collectively confirmed better preservation of the AmnioGraft™ and the fetal tissue product/AM product (32%) which preserved HC-HA/PTX3. The cryopreservation method A was used to manufacture AmnioGraft™ and the fetal tissue product/AM product disclosed. The finding of the loss of HC-HA/PTX3 in AmbioDisk® resembled that reported for a dehydrated human amnion/chorion membrane when a similar heat dehydration process followed by terminal sterilization was utilized (Cooke M, et al. Comparison of cryopreserved amniotic membrane and umbilical cord tissue with dehydrated amniotic membrane chorion tissue. J Wound Care. 2014; 23: 465-474, 476; Tan E K, et al. Structural and biological comparison of cryopreserved and fresh amniotic membrane tissues. J Biomater Tissue Eng. 2014; 4: 379-388). Because terminal sterilization was not included in manufacturing XcellerEYES™, it is possible that the dehydration method by lyophilization applied to XcellerEYES™ degrades the AM sheet product and subsequently degrades HC-HA/PTX3 therein.

### Example 4: Effect of Terminal Sterilization on the Potency of HC-HA/PTX3 the AM Product

Next, an assessment was carried out to assess whether the cryopreservation method A used to process the fetal tissue or AM tissue and/or fetal tissue product herein influenced the preservation of the HC-HA/PTX3 complex to affect the biological properties of the tissue. Furthermore, although the macromolecules that make up the HC-HA/PTX3 complex were preserved indicating HC-HA/PTX3 was preserved per western blot analysis (FIG. 3A-FIG. 3B, FIG. 4A-FIG. 4C), the significant reduction of total HA content, and degradation of HA to a smaller MW species by terminal sterilization (FIG. 2B) in the fetal tissue product/AM product, raised concern over whether the biological properties of the tissue were preserved. To resolve these concerns experimentally, the potency of the fetal tissue product/AM product, relative to the potency in the commercial AM sheet products was investigated by comparing potency of different AM sheet products. About 25 mg/mL of amniotic membrane extract (AME) from each AM sheet product was added in a NO assay taking advantage that system to measure that the inhibitory activity specific to HC-HA/PTX3 was preserved (FIG. 2A; FIG. 2B).

Results: Compared to the inhibitory activity mediated by 20 mg/mL of internal reference material (IRM), the inhibitory activity of AME from AmnioGraft™ was 100%, the inhibitory activity of AME from the fetal tissue product/AM product disclosed was 97% while that of AME from XcellerEYES™ was 14%. There was no detectable NO inhibitory activity in the AME from Ambio2®/AmbioDisk® (FIG. 4D). Collectively, these results confirmed that cryopreservation method A used herein effectively preserved HC-HA/PTX3 and the biological (anti-inflammatory) property thereof. Because HC-HA/PTX3 anti-inflammatory activity was preserved in the fetal tissue product/AM product despite significant reduction of total HA content and lower HA MW, this study suggested that terminal sterilization, which is used to manufacture the present fetal tissue product or AM product, selectively degraded free HA but did not degrade HA that was bound in the HC-HA/PTX3 complex. Because terminal sterilization is not used to manufacture XcellerEYES™, the dehydration method per lyophilization that is used in manufacturing XcellerEYES™ could have reduced HC-HA/PTX3 per the western blot analyses and the reduced associated potency of XcellerEYES™ as measured by the NO assay (FIG. 4D).

The present comparative assessments focused on four cryopreserved amniotic membrane commercial sheet products with different manufacturing processes. It is emphasized that the present study did not conduct comparative studies of AmnioGraft™ or fetal tissue product/AM product on all existing, or commercially available amniotic membrane sheet products. Additionally, these studies did not investigate non-sheet amniotic membrane products or derivatives e.g., amniotic fluid The studies disclosed further stress the point that HC-HA/PTX3 explains the molecular mechanism for AM's biological properties and as such, a tissue product manufacturer can develop an effective manufacturing process, which on one hand meets the minimal manipulation criteria as a 361 HCT/P, and on the other hand ensures safety and maintains the desirable biological properties in any biological tissue product.

This study utilized cryopreservation method A (see previous section above for protocol) where the tissue processing preserves biological properties. Conversely, AM tissue products such as the AM sheet products that were used in the comparative studies utilize different tissue processes e.g., the process of manufacturing tissue under tissue dehydration producing dehydrated AM tissue products. Such processes do not manufacture the disclosed fetal tissue product/AM product which comprises a cryopreserved, hydrated (not dehydrated), low molecular weight (MW) hyaluronic acid (HA), with lower total HA content AM product than that of the comparative AM sheet products tested. This study did not address steps to mitigate any effects from processing tissue through dehydration or processes that do not use the cryopreservation method A of the present disclosure to preserve the biological properties of the tissues or tissue products manufactured.

### Example 5: Treatment of an Eye Indication Using the AM Product Adhered to a Contact Lens

Bandage contact lens: The study illustrates utility of the present disclosure comprising a fetal tissue product or AM product. The AM product comprises bioactive ingredients including HC-HA/PTX3. The AM product also contains low molecular weight hyaluronic acid (HA). The AM product disclosed was processed as a non-dehydrated, terminally sterilized AM product. The hydrated or not dehydrated sterilized AM product contains adhesive properties on both sides of the membrane and directly adheres on surfaces e.g., a contact lens or on a corneal surface of the eye. A case study was conducted to demonstrate the ease, utility, and efficacy of the disclosed AM product in treating an eye indication.

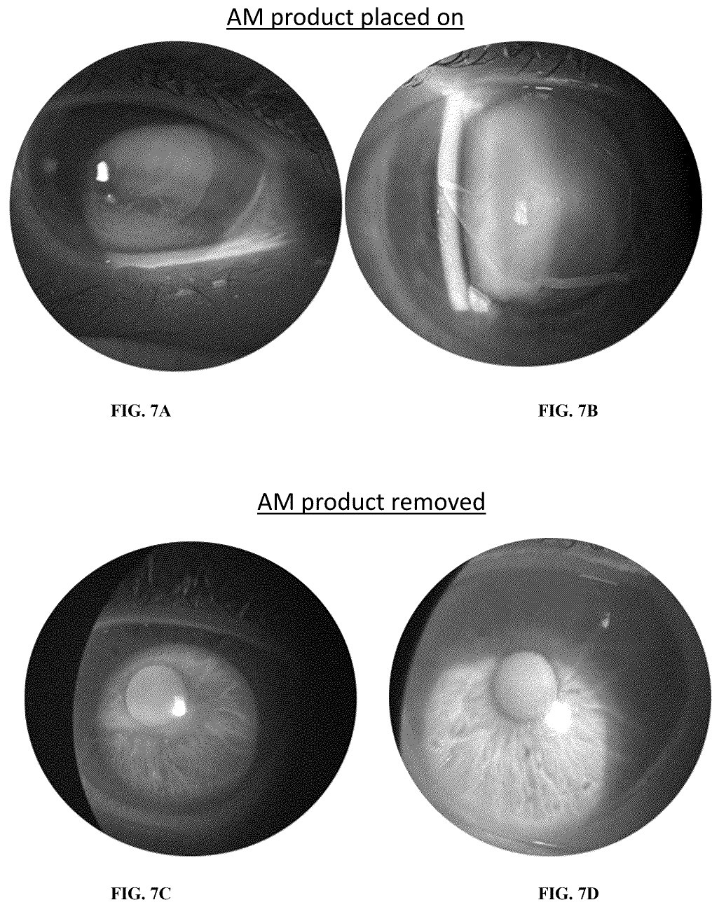

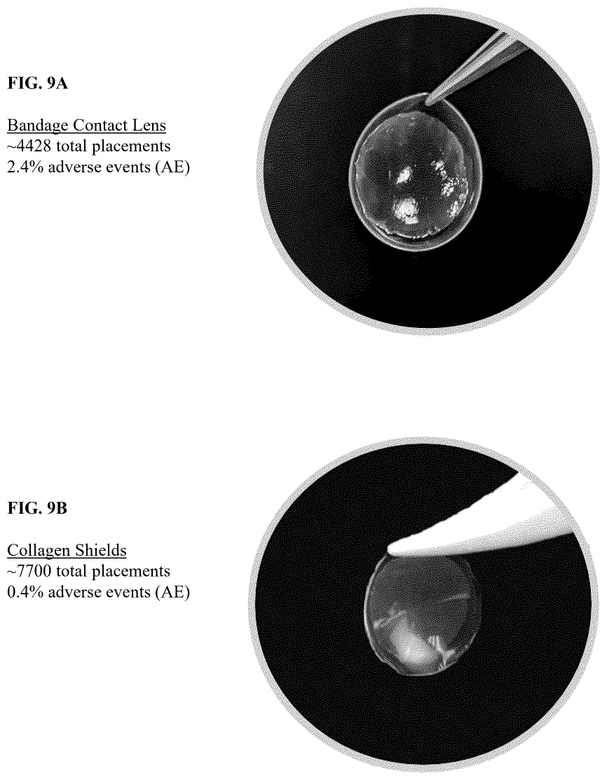

Briefly, a representative female subject presented with moderate superficial punctate keratitis (SPK) and exposure keratitis due to Lagophthalmos FIG. 7A. The subject had not worn contact lenses for >40 years due to persistent ocular dryness and discomfort. To treat the SPK and exposure keratitis due to Lagophthalmos, the disclosed AM product was placed on an adjunctive Acuvue Oasys bandage contact lens (BCL). The BCL was placed on the surface of the eye of the subject for up to 3 days. FIG. 7B. After 3 days, it was determined that (1) there was no AM product in the BCL after removal of the lens; the product had dissolved into the eye. This case study also demonstrated that (2) the AM product adhered to the BCL and to the eye with no other intervention from the investigator for the 3 days; (3) the AM product was efficacious and effective in treating the eye indication as it was confirmed after 3 days that the subject had improved visual acuity, decreased symptoms of ocular dryness, and decreased SPK and/or staining in the area of exposure FIG. 7C-7D. A similar exemplary studied was conducted using both a BCL lens and a collagen shield or collagen corneal shield lens on the eye. A close-up view of the fetal tissue product or AM product adhered on a bandage contact lens (FIG. 9A), or a collagen corneal shield (FIG. 9B) are shown and the number of total placements for each type of lens and adverse events reported with each use is indicated in FIG. 9; both BCL and CS are exemplary types of soft contact lenses and resulted in low amount of adverse events. As shown in FIG. 9A (BCL) or FIG. 9B (CS) a lens with adhered AM product is ready to be placed on the corneal surface of an eye using the same technique for inserting a contact lens (use an index finger); the forceps in FIG. 9 are used for demonstration purposes only and are not meant to be used for insertion.

### Example 6: Biochemical Characterization of Biological Properties of Different Amniotic Membrane Products

Biochemical characterization and potency of HC-HA/PTX3: The biological properties of fetal tissue comprises various biological products that are preserved through the processing and preservation of a fetal tissue product described herein. One example of a biological property that is preserved in a fetal tissue product described includes potent activity of an HC-HA/PTX3 found in a fetal tissue product described herein. HC-HA/PTX3 complex in a fetal tissue product described herein is, at least in part, responsible for the anti-inflammatory/potency, or nitric oxide (NO) inhibitory action of a fetal tissue product. The present study investigated the preservation and potent activity of an HC-HA/PTX3 complex in a fetal tissue product described, e.g., an AM product of the present disclosure. Additionally, studies were performed to show if the other commercial AM sheet products tested did or did not comprise potency actions, and associated HC-HA/PTX3 activity. Effect on the potency of the complex could signal that the tissue product processing steps and/or preservation methods affected the biological activity of HC-HA/PTX3. An internal reference material (IRM) was obtained by biochemically purifying HC-HA/PTX3 from the cryopreserved AM. The purification went through four consecutive rounds of ultracentrifugation in a cesium chloride density gradient containing 4M GnHCl and was used as Internal Reference Material, IRM, (protocol below).

Preparation of HC-HA/PTX3 as IRM and AME: HC-HA/PTX3, biochemically purified from cryopreserved Amniotic Membrane (AM), was used as the Internal Reference Material (IRM). Each frozen AM was extracted (1:1 ratio, v/w) in phosphate-buffered saline solution (PBS; pH 7.4) for 1 hour at 4° C., and the extract was centrifuged at 48,000×g for 30 min at 4° C. The water-soluble AM extract from the six donors was pooled and purified by four consecutive rounds of ultracentrifugation in a Cesium Chloride (CsCl) gradient in 4 mol/L GnHCl/PBS (pH 6.35) at 30,000 rpm for 66 hours at 15° C. After the first three runs, fractions 1-2 (1 mL/fraction) were discarded and fractions 3-12 were collected for the next ultracentrifugation run. After the fourth run, fractions 1-2 were discarded and fractions 3-9 were collected. The purified extracts (Fractions 3-9) were dialyzed in distilled water with 0.5 mM PMSF for 48 hours at 4° C., then aliquoted and lyophilized.

AM Extract (AME): Amniotic membrane extract from each product, each with the surface area of 32 cm², was hydrated by 10 ml of cold PBS for 1 min, pulverized by FreezerMill and extracted (1:1 ratio, v/w) in PBS for 1 hour at 4° C., and the homogenate was centrifuged at 21,130 g at 4° C. for 15 min. The supernatant was collected as amniotic membrane extract (AME).

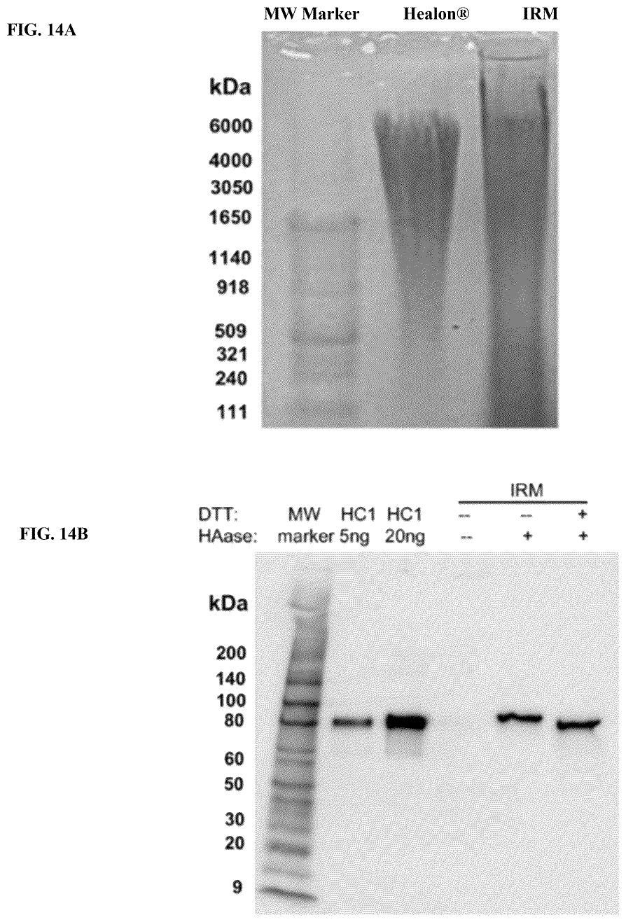

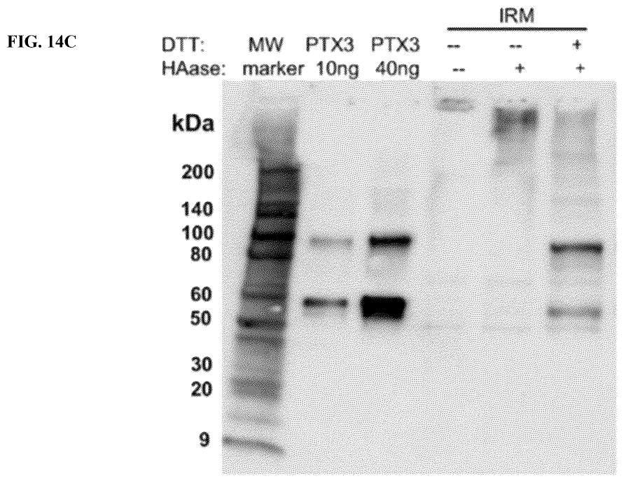

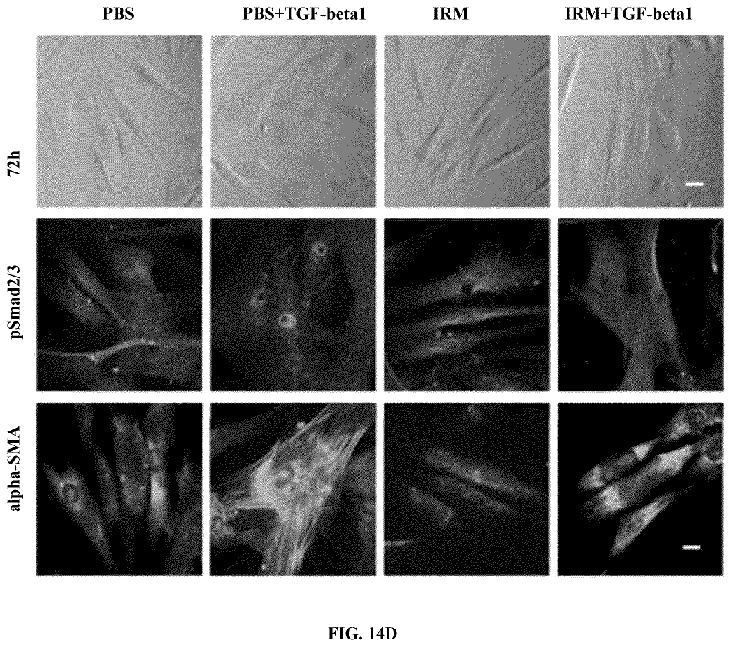

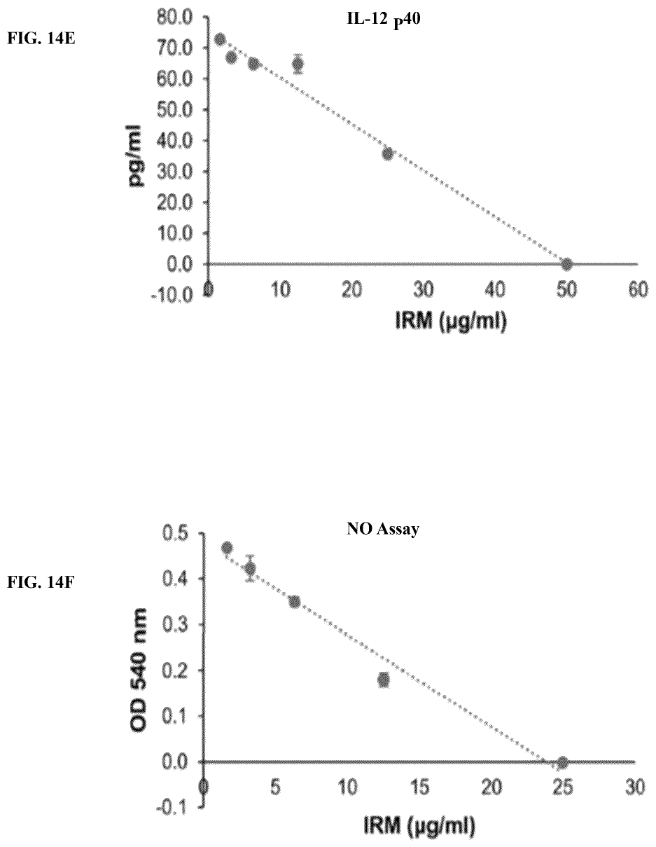

Characterization and potency of the internal reference material (IRM): Agarose gel electrophoresis was used to demonstrate that IRM contained high molecular weight (MW) hyaluronic acid, HA (FIG. 14A), Healon® is a commercial proprietary product used for control purposes Western blot analysis showed that HA was covalently linked with heavy chain 1 (HC1) of inter-α-trypsin inhibitor, and complexed with octameric pentraxin 3 (PTX3) as judged by the release of HC1 by hyaluronidase digestion (FIG. 14B) and PTX3 by hyaluronidase digestion and further cleavage of disulfide bonds therein by DTT to dimer (90 kDa) and monomer (45 kDa) (FIG. 14C). IRM exerted an anti-scarring action by inhibiting TGF-I31-mediated canonical signaling judged by precluding nuclear translocation of pSMAD2/3 and by preventing alpha-SMA expression during myofibroblast differentiation in cultured human corneal fibroblasts with and without TGF-I31 stimulation (FIG. 14D). Because IRM is capable of polarizing proinflammatory M1 macrophages to anti-inflammatory M2 macrophages, 8, 10 IRM dose-dependently inhibited production of interleukin 12, IL-12, (FIG. 14E) and nitric oxide, NO (FIG. 14F) by LPS and IFN-g stimulated proinflammatory M1 macrophages. These results collectively support the IRM as an internal control during biochemical characterization to show the presence and quantity of HC-HA/PTX3 and in the cell-based potency assay of the anti-inflammatory property mediated by HC-HA/PTX3. Because measurement of either NO or IL-12 is comparable, the NO assay was used to compare the potency of the four commercially available AM sheet products.

Filtration via a Nylon Filter: AME from the cryopreserved fetal tissue product/AM product was filtered through the following three procedures, (i) pre-wetting, (ii) filtration, and (iii) recovery. In brief, the filter membrane was completely pre-wetted by passing the filter with 0.5 ml of water (Thermo-Fisher, cat #10977015, pH 6-8, Waltham, MA) and leaving the remaining water for a minimum of 5 min at room temperature. A volume of 0.333 ml of amniotic membrane extract (AME) without filtration was saved as the control. Next, about 0.333 ml of AME (96 μg/ml) was forced to filter through a nylon filter (Pall #AP-4436: pore size about 0.2 m; filtration area of about 3.9 cm²) with a 5-ml syringe and the filtrate was collected. After filtration, about 0.2 ml of water was forced to pass through a filter via a syringe. After gently tapping for about 1 min and slowly pushing the syringe back and forth 10-15 times, the solution left on the top of filter was collected by back sucking with air using a new empty 5-ml syringe. The wash was repeated once, and a sample was collected as the recovered fraction which contained the HC-HA/PTX3 complex.

Nitric oxide (NO) assay: After establishing IRM as the internal control, the next experiment was conducted to ensure that AM extract (AME) from the AM sheet product was subjected to biochemical characterization and a NO assay to measure activity of the HC-HA/PTX3 complex. To assess the suitability of the system, a novel filtration method was employed. The filtration method selectively captures HC-HA/PTX3 via a nylon filter and separates it from the AME. The AME is retained in the filtrate. The concept was confirmed by the observation that IRM solubilized in water or saline was selectively captured by the nylon filter following filtration, since only 2.5% of the added IRM was lost in the filtrate (not shown). Therefore, the nylon filter (filtration) method selectively captured HC-HA/PTX3 in AME that was obtained from cryopreserved AM product.

Polarization into M2 Macrophages Measured by NO Assay and IL-12 Assay: HC-HA/PTX3 was biochemically purified (protocol above) from cryopreserved AM through four consecutive rounds of ultracentrifugation in a cesium chloride density gradient containing 4M GnHCl and was used as Internal Reference Material (IRM). To conduct the NO assay and IL-12 assay, RAW264.7 cells expanded on plastic in αMEM+10% FBS were digested by cell dissociation buffer upon 70-80% confluence and seeded at 3×10⁴ cells per cm² on plastic in αMEM+10% FBS. After 16 hours (overnight), cells were treated with an internal reference material (IRM) at 1.6 μg/ml, 3.2 μg/ml, 6.3 μg/ml, 12.5 μg/ml, 25 μg/ml and 50 μg/ml of HA in αMEM+10% FBS (M2 assay), with IRM at 1.6 μg/ml, 3.2 μg/ml, 6.3 μg/ml, 12.5 μg/ml and 25 μg/ml of HA in αMEM+10% FBS (NO assay), with recovered sample and filtrate from AME filtration at 12.5 μg/ml, 25 μg/ml and 50 μg/ml of hyaluronic acid, HA, in comparison to 20 μg/ml of IRM as the control in αMEM+10% FBS, or with 25 μg/ml of AME product extract. After a further 16 hours (overnight), cells were treated with 1 μg/ml of LPS and 200 U/ml of IFN-γ except the negative control group. Next, the medium was tested using NO assay kit (protocol see Sigma-Aldrich, St. Louis, MO) and/or Mouse IL-12 p40 ELISA Kit (R&D Systems, Minneapolis, MN).

Statistical Analysis: All data were shown as means±SD for all groups using ANOVA and the Student's paired and unpaired t test was conducted. The results were demonstrated as two-tailed p-values, with p<0.05 indicating statistically significant differences.

Western Blot Analysis: IRM or AME filtration samples were loaded with 5 μg of HA per lane and AME from four different commercial AM sheet products were loaded with 2 μg of HA per lane with or without hyaluronidase (HAase) treatment at 37° C. for 1 h. Thereafter, samples were denatured in Laemmli Buffer (1:1 dilution) at 95° C. for 5 min and electrophoresed on 4-15% (w/v) gradient acrylamide gel in 1×Tris/Glycine/SDS Buffer at 100 V for approximately 1.5 hours and transferred to nitrocellulose membrane. The membrane was blocked with 2% bovine serum albumin (BSA) in TBST (50 mM Tris-HCl, pH 7.5, 150 mM NaCl, 0.05% Tween 20) followed by washing (standard procedure) and incubation of the membrane in primary antibodies against heavy chain 1 (HC1) of inter-α-trypsin inhibitor, or PTX3 (1:1000) in 2% BSA in TBST overnight at 4° C. with a rotator (Maxi Rotator, Labline). After three washes for 10 minutes in TBST, the membrane was incubated with specific HRP-conjugated secondary antibody (1:1000) in 2% BSA in TBST for 1 hour (1 h) at room temperature followed by three washes for 10 min each in TBST. Protein bands were detected and analyzed with Image Analyzer (LI-COR imaging system, LI-COR, Inc, Nebraska).

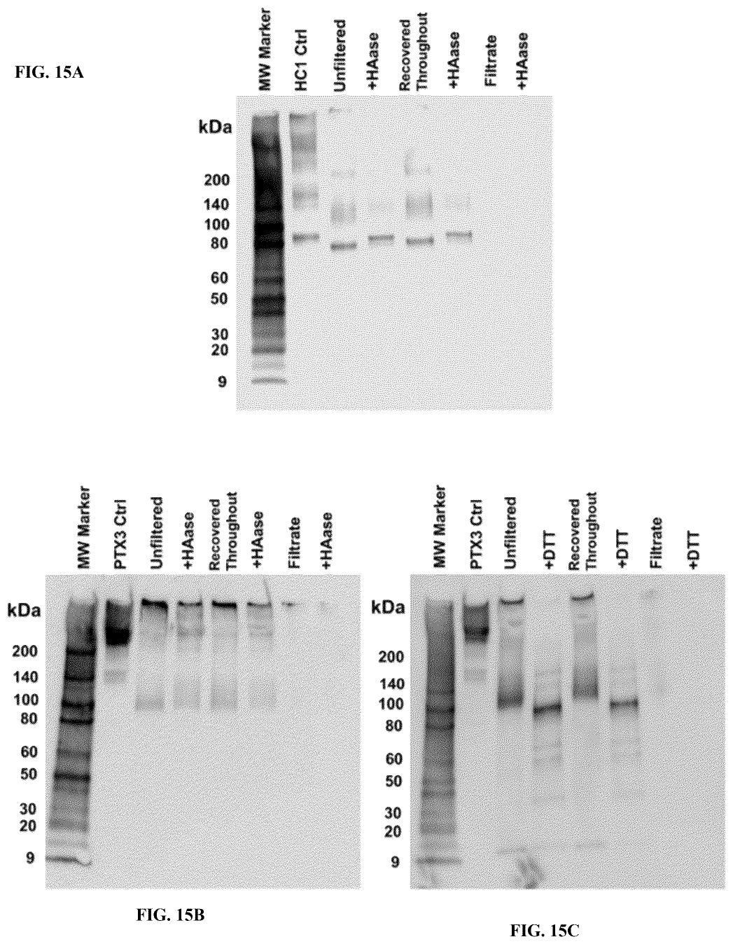

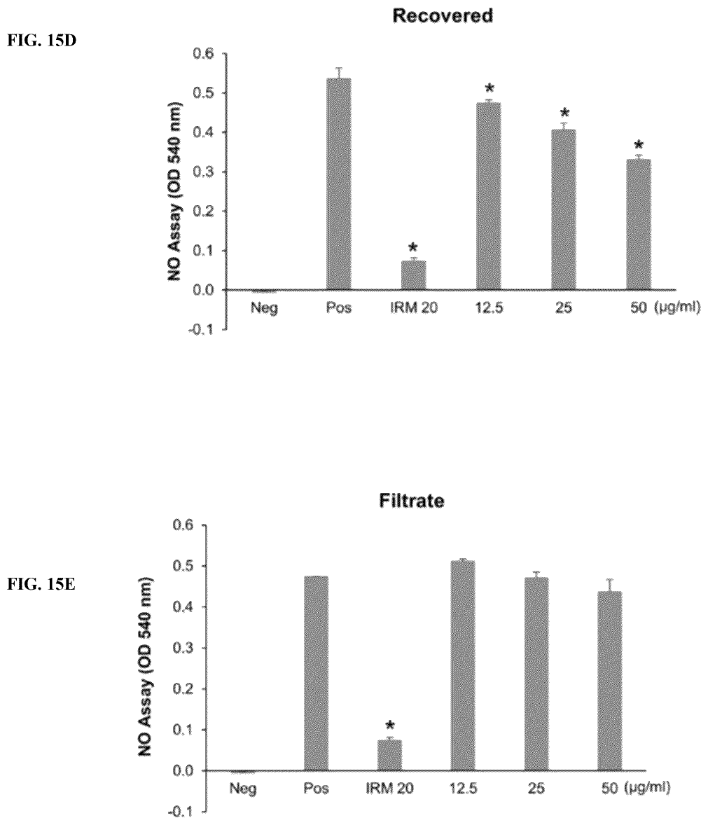

Results: Densitometry of western blot of HC1 released by hyaluronidase digestion (FIG. 15A) showed only a 2.7% loss of HC-HA/PTX3 in the filtrate when compared with the unfiltered AME. Densitometry of western blot of high molecular weight (HMW) smear of PTX3 released by hyaluronidase (HAase) digestion also showed a similar finding (FIG. 15B). To circumvent the challenge of quantifying a smear, densitometry of western blot of PTX3 dimer released by hyaluronidase digestion plus reduction by dithiothreitol (DTT) showed a 1.1% loss of HC-HA/PTX3 in the filtrate when compared with the unfiltered AME (FIG. 15C). After rinsing with water, 75% of the captured HC-HA/PTX3 could be recovered. The recovered sample and filtrate were subjected to a NO assay. The recovered sample (FIG. 15D), but not the filtrate (FIG. 15E), dose-dependently inhibited the production of NO by LPS and IFN-g stimulated proinflammatory M1 macrophages in the NO assay (when compared with IRM (the internal control). Collectively, these results confirmed that AME derived from cryopreserved AM was suitable to be subjected to the NO assay to measure the inhibitory activity specific to HC-HA/PTX3.

Potency of HC-HA/PTX3 in the amniotic membrane extracts (AME) from the aforementioned amniotic membrane (AM) sheet products, AmnioGraft™, XcellerEYES™, Ambio2® (or AmbioDisk®), and the fetal tissue product or AM product disclosed was determined to assess any differences in the potency of the biologically active HC-HA/PTX3. For example, the different processing and preservation methods used to obtain any of the commercial AM sheet products (Table 3) could affect potency of the HC-HA/PTX3 complex. The AME products from the different AM sheet products were subjected to biochemical characterization and nitric oxide (NO) assay to measure the potency of HC-HA/PTX3. A novel filtration method which selectively captures HC-HA/PTX3 complex and separates it from the AME filtrate was tested. To confirm system suitability, the novel filtration method, which uses a nylon filter to separate HC-HA/PTX3 from the AME, was employed (the isolation of the IRM has been reviewed in the previous section). Briefly, HC-HA/PTX3 present in the AME would be captured in the nylon filter and recovered while the separated AME would remain in the filtrate. It was confirmed that the internal reference material (IRM) solubilized in water or saline was selectively captured by the nylon filter following the filtration step, as only 2.5% of added IRM was lost in the filtrate (data not shown).

Expansion of Human Corneal Fibroblast Cells (HCF) and TGF-β1 treatment on Different Substrates: Passage 3 (P3) human corneal fibroblast (HCF) cells expanded on plastic in DMEM+10% FBS were digested by 0.25% Trypsin/EDTA upon 80-90% confluence and seeded at 5×10³ cells per cm² on cover slip in DMEM+10% FBS. After 24 h, all wells were switched to a serum-free medium for another 24 hours when 20 μg/ml of the internal reference material (IRM) in DMEM+ITS was added with or without 10 ng/ml of TGF-beta 1 (TGF-β1) for 72 hours.

Immunofluorescence Staining: After HCF cells were treated with 20 μg/ml of IRM with or without 10 ng/ml of TGF-β1 for 24 or 72 hours, cells on cover slips were fixed with 4% paraformaldehyde for 20 min followed by permeabilizing with 0.3% Triton X-100 in PBS for 30 min and then blocked with 2% BSA in PBS for 1 hour. Next, cells were incubated with specific primary antibodies at 4° C. for 16 hours overnight. After 3 washes with PBS, cells were incubated with the secondary antibodies for 1 hour at room temperature. Cells were washed three times with PBS, and the nucleus was stained with Hoechst 33342 for 5 min before being analyzed with confocal microscope (LSM700; Carl Zeiss, Thomhood, NY).

Quality control employed: The present studies show cryopreservation method A achieves effective preservation of HC-HA/PTX3 complex and does not affect the associated biological properties of the AM tissue. Terminal sterilization can cause degradation of HA to a smaller MW HA, which displays a pro-inflammatory response. To mitigate this unwanted effect, terminal sterilization performed in ice but not in a liquid state (ice used in cryopreservation method A) prevents HA degradation in the HC-HA/PTX3 complex. Cryopreservation method A was used to manufacture and/or process the fetal tissue product/AM product disclosed here. Tissue processing that uses dehydration by heat of lyophilization by other tissue processing/manufacturing facilities may affect presence, quantity, and potency mediated by HC-HA/PTX3.

### Example 7: Clinical Efficacy Testing

The present treatment study was conducted to determine the short-term benefits of various ocular diseases or conditions. An exemplary dataset from the study is provided which shows outcomes when AM product was used as a treatment in less severe forms of ocular surface disease (OSD), including mild to moderate DED and stage 1 neurotrophic keratitis (NK).

Methods: The AM Product described herein was used by physicians throughout the US. Study subjects or patients were enrolled in the treatment study and confirmed consent to participate in the six-month study period. After each use, study subjects or patients (terms used interchangeably) completed a survey to indicate the experience. Exemplary questions that were asked related to their experience with product handling and application, comfort level during treatment, and short-term outcomes. Survey results were analyzed; continuous data were reported as mean±standard deviation or median and range, and categorical data were described using frequency and percentage.

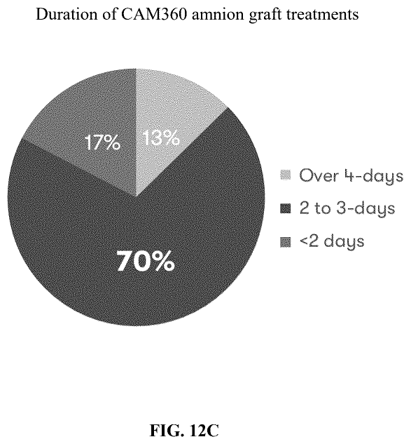

Results: A total of 271 positive surveys were received and analyzed based on the experience of 126 physicians who used the AM product or fetal tissue product or AM product during the study period. Most of the study subjects or patients (80%) were over the age of 55 years, which is consistent with the notion that OSD is more prevalent in older adults (FIG. 12A). Among ocular conditions treated, 66% of cases had dry-eye-disease (DED), 67% had superficial punctate keratitis (SPK) while 47% had neurotrophic keratitis (FIG. 12B) was detected Most patients did not have a history of contact lens use; only 33 out of 271+(12%) subjects wore contact lenses prior to the present treatment study. Common co-morbidities were diabetes and glaucoma (18%; 1+8 out of 271+), hypertension (12%; 33 out of 271+), Arthritis (7%; 20 out of 271+), high cholesterol (5%; 15 out of 271+), and Sjogren's disease (1+%; 11 out of 271+).

Application: The fetal tissue product, or AM product had a wide application in treating ocular diseases or conditions. For example, subjects received treatment who had dry-eye-disease (DED) (including glaucoma medication-induced DED), superficial punctate Keratitis (SPK), or neurotrophic keratitis (NK). Overall, 95% of subjects had one or more of ocular conditions, for example, one subject having DED, SPK, and NK conditions in their eyes. Other applications included use of AM product to treat non-healing injury/wound (<1%), post debridement (1+%), epithelial basement membrane dystrophy (EBMD) (<1%), and recurrent corneal erosion (<1%).

Product Application: The significant reduction of total HA content and notable reduction of the MW of HA) in the fetal tissue product or AM product explains why the product attaches well to the CS or BCL for in-office application because of an increasing adhesive property due to the significant reduction of total HA, which facilitates lubrication. The AM product was applied onto the ocular surface in conjunction with a Collagen Shield (CS) in 74.5% of cases, or a Bandage Contact Lens (BCL) in 25.2% of cases. Only one case utilized a Contour Lens with an AM product. Overall, the fetal tissue product or was applied without difficulty in 83% (228 out of 274) of cases. During the application process, some difficulty was noted in 17% ( 46/274) of cases as it related to removing the fetal tissue from its packaging, or removing the backing paper from the tissue (22% or 10/46), difficulty handling or visualizing the tissue (33% or 15/46), difficulty placing the lens and/or tissue on the eye (30% or 14/46), and issues with the lens itself (11% or 5/46), or unspecified ( 2/46).

Using the AM Product with bandage contact lens, BCL: Clinical application of AM product with BCL is recommended for mild-to-moderate), superficial punctate Keratitis (SPK), dry-eye-disease (DED), or neurotrophic keratitis (NK) stage 1, and reduced corneal sensitivity. There have been approximately 4,428 total the AM product+BCL; placements on the eyes of subjects or patients in this project. Of these only about 2.4% of the subjects or patients experienced adverse events (AE). The following steps are recommended for best practices and optimal comfort while using the AM product with BCL to obtain this low level of AE. the AM product/BCL duration of treatment being up to 48 hours, (1) use of sterile conditions e.g., wearing clean gloves to mitigate the risk of introducing infectious agents at time of BCL placement, (2) optionally anesthetize the eye e.g., apply a drop of anesthetic prior to placement to minimize any initial foreign body sensation, (3) use prophylactic antibiotics drops on the ocular surface prior to placement of the AM product loaded BCL, (4) optionally, use taping method—after placement, the eye may be taped to reduce potential exposure-related dryness, specifically during sleep, use of nictavi patch or tape tarsorrhapy may be employed, (5) use preservative-Free Artificial Tears—the patient should apply preservative-free artificial tears every 30 minutes to one hour during treatment, before going to sleep, and upon waking up to avoid the BCL drying, also avoid rubbing the eye during treatment, avoid direct water exposure to the eye during treatment, avoid ocular exposure to aerosols, hairsprays, cosmetics, lotions and such during treatment, (6) use concomitant medications is okay except topical steroid, (7) watch out for air bubbles—examine for and remove any air bubbles after insertion and if air bubbles persist, ensure the patient follows the preservative-free artificial tear regimen noted above, (8) this treatment is not recommended also for subjects or patients with allergies e.g., patient has a prior history of HSV/HZO, floppy eyelids, or uses a CPAP, this treatment may not be suitable or if patient presents with any eye defects or ulcers. In the even a patient experiences worsening pain, redness, discharge, excessive tearing, or swelling during use, please contact healthcare provider. If the treatment duration will exceed 48 hours, a prophylactic antibiotic drop e.g., polytrim (TID) is recommended for the duration of treatment.

Using AM product with a collagen shield or collagen corneal shield (CS): Clinical application of AM product+CS is recommended for mild-to-moderate SPK/DED, and for neurotrophic keratitis stage 1. There have been approximately 7,700 total PAM+CS placements on the eyes of subjects or patients in this project. Of these only about 0.4% of the subjects or patients experienced adverse events (AE) while using CS product. The recommended for best practices including sterility and optimal comfort while using AN AM product disclosed with CS are like those for BCL above. The recommended duration of AM product with CS treatment is up to 72 hours. To apply the treatment, anesthetize the eye, apply a drop of anesthetic prior to placement to reduce the potential for discomfort associated with the low pH of the CS. Antibiotics is recommended as is 2-3 drops of saline to ensure complete hydration of the CS. Treated eye should remain taped between 48-72 hours and avoid rubbing eye during treatment or direct exposure to water or aerosols, lotions during treatment. Taped eye should be closed prior to removal of tape and preservative-free artificial tears should be applied three times daily thereafter. Additionally, a post-insertion evaluation step can be instituted to check and confirm proper centration of the lens and the AM product over the cornea. Allergies should be monitored including any temporary stinging sensation following insertion of the CS, but this should neutralize quickly. The CS application method is not recommended for subjects or patients with known allergy to collagen or bovine product. CS wear time should be limited to 72 hours to avoid infectious agent that may arise from prolonged wear time. Patients with ocular defects, ulcers, epithelial basement membrane dystrophy (EBMD) or recurrent corneal erosion (RCE) should not use this product. Use of the AM product with CS is recommended for subjects with any ocular indication including for example, dry-eye-disease (DED), neurotrophic keratitis (NK), or reduced corneal sensitivity. Subjects or patients with known allergy to collagen or bovine products should not receive collagen shield treatment. In the even a patient experiences worsening pain, redness, discharge, excessive tearing, or swelling during use, please contact healthcare provider.

Wear time: The wear time for the fetal tissue product or the AM product varied from 1 to 7-days, with one survey noting a 2-week wear time, resulting in an overall average wear time of 2.5-days (1.5). The most common duration of treatment time known as wear time was 2 to 3-days, which occurred in 70% of all the AM product cases (FIG. 12C). The AM product applied with a CS was generally worn for on average for 2.4-days (±2.3) while the AM product was applied with a BCL on an average for 2.8-days (±0.99). In cases where CSs were used with the AM product, 79% (161 out of 204) had a treatment duration of 2 to 3-days. About 12% (25 out of 204) had a duration of wear less than 2-days, and 9% (18 out of 204) of cases reported follow-up at over 4-days. In cases where BCLs were used, 32% (22 out of 69) had a <2-day duration, 43% (30 out of 69) had a 2 to 3-day duration, and 25% (17 out of 69) had over a 4-day duration.

Product Dissolutions: The fetal tissue product or the AM product was completely dissolved in 45% (122 out of 274) cases at follow-up, which occurred on average in 2.8-days (±1.7). Most cases saw the fetal tissue product or the AM product dissolve 2 to 3-days after application (66%; 81 out of 122). When compared by lens type, both CS and BCL lenses had similar the AM product dissolution rates: 45% (91 out of 204) in the CS group and 43% (30 out of 69) of the AM product in the BCL group. In the CS group, complete dissolution occurred in 2.6-days (±1.12) on average, with 52% ( 13/25) of cases being treated in less than 2-days, 40% (64 out of 161) of cases had a 2 to 3-day treatment duration, and 78% ( 14/18) of cases had an over 4-day treatment duration. In the BCL group, dissolution was observed in 3.8-days (±2.6) on average. Complete dissolution occurred in 9% ( 2/22) of cases with <2-days treatment duration, 53% (16 out of 30) of cases with 2 to 3-days treatment duration, and 71% ( 12/17) of cases with over a 4-day duration.

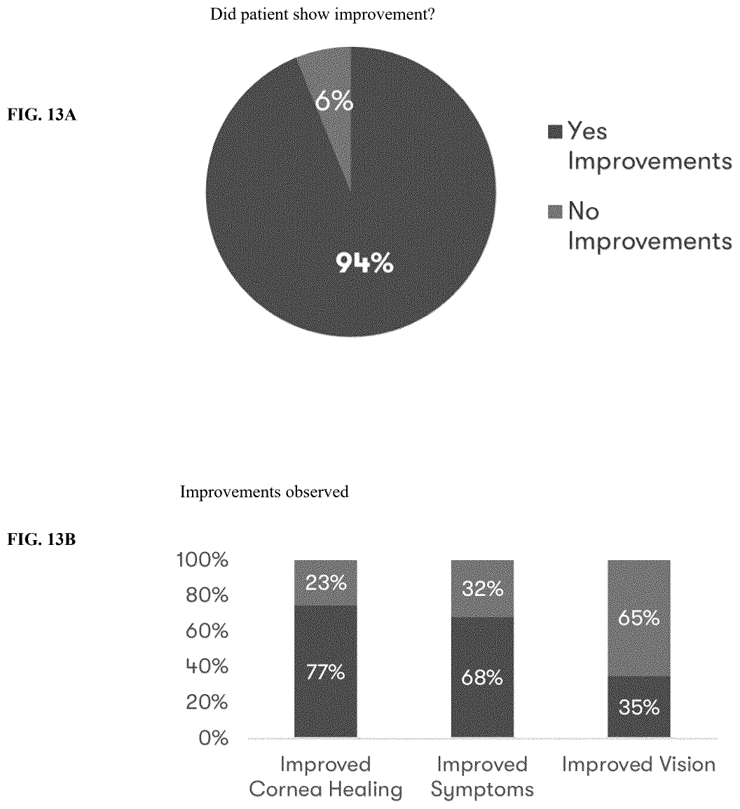

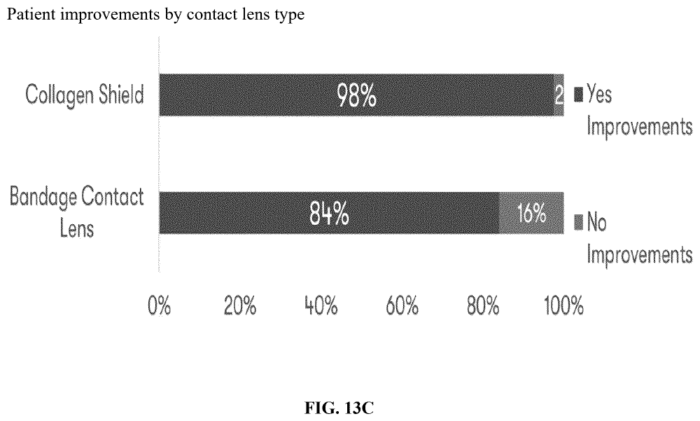

Observed Improvements: Over 94% (258 out of 274) of study subjects or patients who underwent treatment with the fetal tissue product, or the AM product had improvement in one or more ocular conditions (FIG. 13A). For example, (i) corneal healing was observed in 77% (211 out of 274) cases. Of the patients with improved corneal health, corneal signs such as decreased corneal staining/SPK, decreased redness, and improved tear meniscus were seen in 58% (122/211) of cases and completely resolved in 42% (89 out of 211) of cases. (ii) 68% of cases (186 out of 274) noted an improvement in symptoms which included reduced dryness, pain, and foreign body sensation (74%; 138 out of 186), or a complete resolution of symptoms (26%; 48 out of 186). (iii) Improvement in visual acuity (81%; 77 out of 95) or decreased blurry vision (16%; 18 out of 95) was noted in 35% (95 out of 274) of cases (FIG. 13B).

98% of patients (199/204) noted improved signs and/or symptoms after the AM product treatment with a collagen shield or collagen corneal shield (CS), while 84% of patients (58 out of 69) noted improved signs and/or symptoms after the AM product treatment with a bandage contact lens (BCL; FIG. 13C). 80% (163 out of 204) of CS wearers and 68% (47 out of 69) of BCL wearers had improved cornea healing. 72% (146 out of 204) of CS users and 58% (40 out of 69) of BCL users had improved symptoms. About 35% (72 out of 204) of CS users and 33% (23 out of 69) of BCL user showed improved vision. The CS group saw improved signs or symptoms in 88% ( 22/25) of patients who wore the AM product for <2-day wear, 98% (159/161) for 2 to 3-days wear, and 100% (18/18) for over 4-day wear. The BCL group showed improved signs or symptoms in 77% ( 17/22) for <2-day wear, 90% ( 27/30) for 2 to 3-day wear, and 82% ( 14/17) for over 4-day wear.

Only 6% of subjects or patients (cases) had no improvements after the use of the AM product. Of the 5 the AM product with collagen shield (CS) cases that noted no improvement, 4 out of the 5 patients were non-contact lens wearers, 3 of which noted some discomfort or irritation with the CS and one noted that the AM product may have slipped out of place during wear. For the 5th case, the patient removed the AM product after just 24-hours of wear. For the 11 cases that showed no improvement when the AM product was used with a bandage contact lens (BCL), 9 cases (82%) noted patient complaints.

When assessing subjects or patient improvements in relation to product dissolution, over 98% (120 out of 122) noted improvement when the AM product was completely dissolved. However, improvements were also seen in over 91% (138 out of 152) of cases where the AM product was not fully dissolved, or where dissolution was unknown, with the undissolved group showing improvements in 92% (114 out of 124) of cases, and the unknown group showing improvements in 86% (24 out of 28) of cases.

Safety: 90% of subjects or patients who underwent treatment with the AM product had no complaints. When the complaints were broken down by lens type, 7% ( 14/204) of patients who used the AM product with a CS reported a complaint, 19% ( 13/69) of patients who used a BCL with the product reported a complaint. Complaints for CS wear varied from claims of pain and discomfort (4.4% or 9/204), light sensitivity (0.98% or 2/204), burning upon placement (0.98% or 2/204), and the CS falling out prior to removal (0.5% or 1/204). BCL complaints included pain and discomfort (10% or 7/69), hypopyon (1.4% or 1/69), corneal infiltrates (1.4% or 1/69), epithelial defects (2.9% or 2/69), and corneal ulcers (4.3% or 3/69), with some cases experiencing multiple of the above complaints. The BCL complaint rate was similar across different product wear durations, including <2-days (18.2%), two to three days (20%), and over 4-days (17.6%).

Application and Handling: Most physicians who applied the AM product disclosed to the ocular surface had no difficulty handling and applying the product. Difficulty with the application process of the AM product was observed in 16% of cases, with no significant difference noted when comparing the AM product application with a CS or BCL. Most of the difficulty was observed when removing the AM t tissue from its packaging or removing the backing paper from the tissue. In such cases, physicians generally used loops, or placed over a darker background to help visualize the tissue. Furthermore, grasping the AM product with forceps perpendicular to the pre-cut slit in the tissue backing paper helped transfer and maneuver the product.

Patient Outcomes: After treatment with the AM product, improvements were noted in 94% of cases, of which the majority (95%) of patients had pre-existing DED, NK, and/or SPK. In moderate to severe DED patients, 88% of patients demonstrate an improved ocular surface at 1-week, 1-month, and 3-months. These results suggest potential benefits of the AM product covering a spectrum of DED patients, from mild to moderate the AM product to more moderate to severe (Prokera).

The AM product was noted to dissolve in only 45% of cases, suggesting the product does not need to fully resorb to provide benefit. Similarly, studies evaluating Prokera showed benefit in 88% of patients despite the tissue being fully dissolved in only 42% of cases. It should be noted that dissolution of the tissue has been significantly correlated with the duration of placement on the ocular surface.

6% (16 out of 274) of patients did not show improvement after the AM product treatment, and 11 of these 16 cases (69%) used a BCL during the application process and experienced complaints during wear. The concomitant use of BCL was noted to have a relatively higher complaint rate compared to concomitant CS use (19% vs 7%, respectively). BCLs are known to have adverse effects, such as infiltrates and corneal ulcers, occurring at rates of 3.11% and 1%, respectively. Concomitant topical drops or artificial tears may be recommended during wear of the lens to prevent the BCL from drying out and causing discomfort. Alternatively, with the use of a CS, the most common complaint was discomfort or burning, which may be related to several factors including movement of the CS on the ocular surface, inadequate rehydration prior to placement, failure to invert the CS prior to placement, sensitivity to bovine collagen material, or low pH of the CS (also associated with inadequate rehydration).

Overall and based on data of 274 clinical applications of the AM product from 126 physicians, the AM product may help improve signs & symptoms in patients with mild to moderate DED, NK, and SPK. The AM product was applied with a CS, BCL, or Contour Lens in 74.5%, 25.2%, and <1% of cases, respectively. A treatment duration of 2 to 3-days was the most common, occurring in 70% of cases. After treatment, over 94% of cases saw improvements. 98% of patients noted improved signs and/or symptoms after the AM product treatment with a CS while 84% of patients noted the same after using a BCL. Complete dissolution of the AM product tissue was not necessary to achieve improvements to the patients' conditions.

### Example 7: AM Dissolution Test

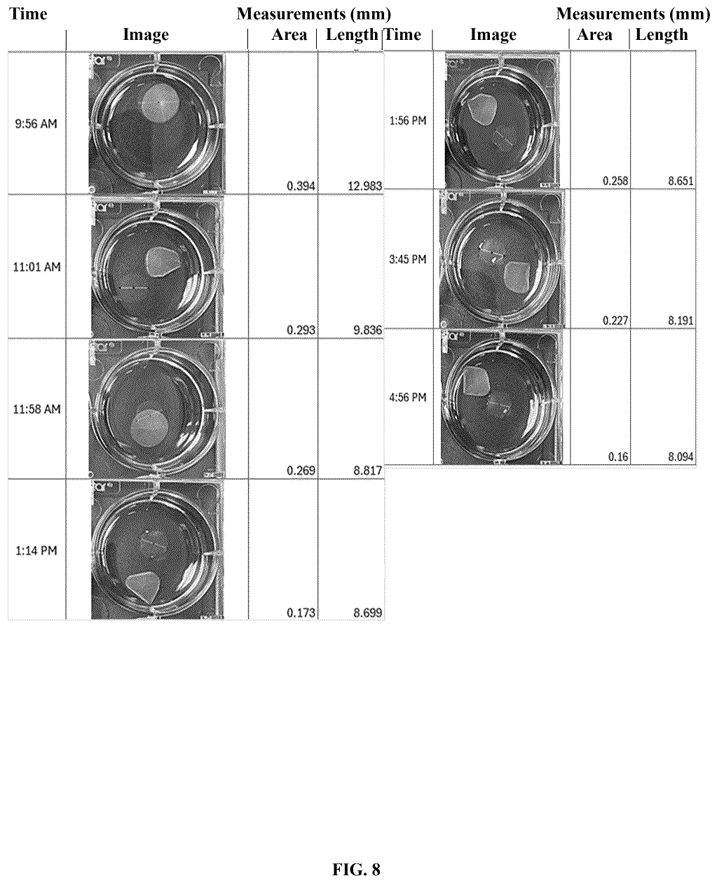

Table 4 shows a preliminary dissolution test of the AM product that has a specific area, 0.394 millimeters (mm) and certain length, 12.983 mm and the changes that occurred with the passage of time. More temporal shrinkage of the tissue product is illustrated in FIG. 8. Table 4 shows overall temporal changes and shrinkage of the AM product which capture tissue dissolution process when the tissue product is adhered to the corneal surface of an eye

TABLE 4

| | | | MEAN AREA | CHANGE | LENGTH | CHANGE IN |
|---|---|---|---|---|---|---|
| | | # | OF CS | OF AREA | OF CS | LENGTH |
| TIME | MINUTE | MEAS | (mm²) | (mm²) | (mm) | (mm) |
| 9:56:00 AM | 0 | 5 | 0.394 | — | 12.983 | — |
| 11:01:00 AM | 65 | 5 | 0.293 | −0.101 | 9.836 | −3.147 |
| 11:58:00 AM | 122 | 5 | 0.269 | −0.125 | 8.817 | −4.166 |
| 1:14:00 PM | 198 | 5 | 0.173 | −0.221 | 8.699 | −4.284 |
| 1:56:00 PM | 240 | 5 | 0.258 | −0.136 | 8.651 | −4.332 |
| 3:45:00 PM | 349 | 5 | 0.227 | −0.167 | 8.191 | −4.792 |
| 4:56:00 PM | 420 | 5 | 0.160 | −0.234 | 8.094 | −4.889 |

### Example 8: AM Product Long-Term Storage

The stability study was conducted to establish long-term storage conditions (˜20° C. freezer maintained between −25° C. to −10° C. and controlled room temperature maintained between 20° C. to 25° C.) for routine storage and accelerated storage conditions (40° C.±2° C./75% RH±5% RH) and to account for customary handling and shipping of which complies with ICH QIA (R2)

The storage conditions and the testing were performed in accordance with ICH guidelines Q1A (R2) for “Stability Testing of New Drug Substances and Products”, used for drug substances and products, to provide (1) evidence on how the storage solution vary with time and temperature and (2) to establish recommended storage conditions and expiration dates of the fetal tissue product or AM product. The critical quality attributes (CQA) tested for the fetal tissue product or AM product were (a) hematoxylin and eosin (H&E) staining and (b) container closure integrity testing (CCIT).

The samples were stored at long-term storage conditions (in e.g., a controlled freezer) at temperature of −20° C.±5° C., and constant climate chambers (CCC) set at 25±2° C., 60±5% relative humidity (RH). The accelerated samples were stored in CCC set at 40° C.±2° C./75% RH±5% RH. (iv). Sampling forms were compiled, environmental conditions were monitored, and recordings were obtained. The samples were stained with H&E. The scope of the study was for the six- and nine-months stability storage testing under (1) long-term storage conditions (controlled freezer conditions minus 20 degrees centigrade) −20° C.±5° C., (2) long-term storage conditions (CCC) of 25±2° C., 60±5% RH, and (3) accelerated storage conditions (CCC) of 40° C.±2° C./75% RH±5% RH. The six months and nine (270 days) months pull for accelerated storage conditions (40° C.±2° C./75% RH±5% RH) represented 18 months and 2 years of storage at the long-term storage conditions (−20° C. freezer maintained between −25° C. to −10° C. and controlled room temperature (CCC) maintained between 20° C. to 25° C.), respectively.

Results: As indicated above, one lot of fetal tissue product or AM product was stored for six and nine months of stability storage under long-term storage conditions of (1) −20° C.±5° C., long-term storage conditions of (2) 25±2° C., 60±5% RH, and (3) accelerated storage conditions of 40° C.±2° C./75% RH±5% RH.

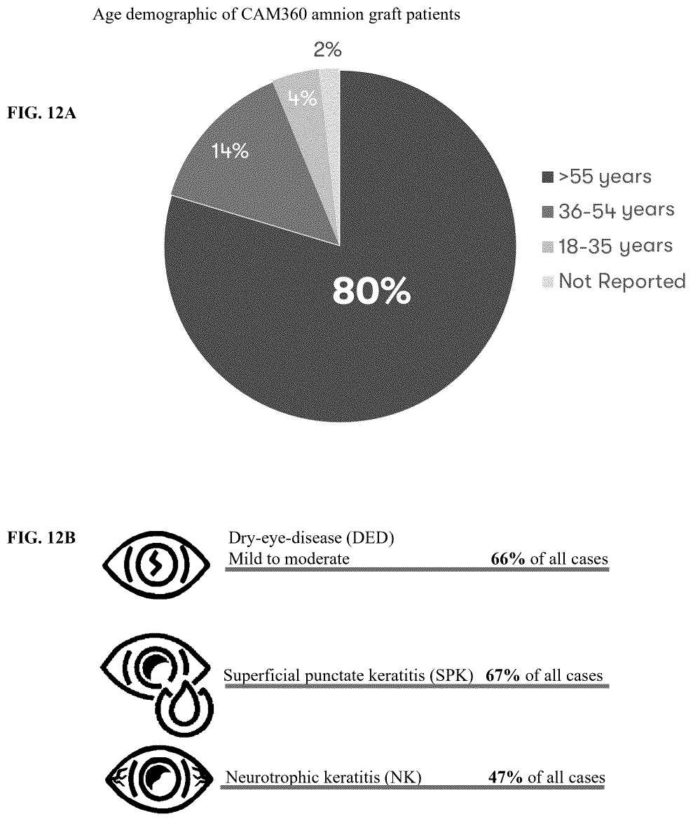

The H&E Staining at the baseline showed that the tissue presented a continuous presence of a thin basement membrane sandwiched between a simple epithelium and stroma. The 6- and 9-month results demonstrated that the tissue was able to maintain integrity during the long-term and the accelerated conditions, which was demonstrated using the H&E staining. At the 3-month (FIG. 11A) and 6-month (FIG. 11B) stability or pull time point the AM tissue was fully hydrated with excess saline solution remaining in the inner pouch, while the solution within the pouch started shrinking at the 9-month timeline (FIG. 11C). For safety testing, Container Closure Integrity Testing (CCIT) for primary and secondary packaging was tested per ASTM-F88 and ASTM-F2096. The seal strength met the criteria of NLT 1 lbf/in and no leaks/bubble for the bubble emission test during the nine-months of stability for long-term storage conditions of (1) −20° C.±5° C., long-term storage conditions of (2) 25±2° C., 60±5% RH, and (3) accelerated storage conditions of 40° C.±2° C./75% RH±5% RH storage conditions, which means that the containers were able to maintain product sterility.

Accelerated Aging Time (AAT) calculation: The proposed storage condition for Matrix P is Controlled Room Temperature defined as 20° C. to 25° C. per USP <659>. Therefore, the shelf life was calculated using the storage temperature setpoint of 23° C. (based on the midpoint of controlled room temperature). After nine months of storage at (3) accelerated conditions, there was separation of the tissue from the backing during the H&E Staining, therefore, the shelf life was calculated using the six-month data during which it was determined that the stability of the samples was maintained. The six-month stability pull date was conducted after 181 days.

$$AA T = \frac{Real Time ( RT ) aging period}{{Q}_{10}^{[ \frac{{T}_{A A} - {T}_{R T}}{10} ]}} \to \frac{Real Time ( RT ) aging period}{}$$

$$RT aging period = AAT * {Q}_{10}^{[ \frac{{T}_{A A} - {T}_{R T}}{10} ]} = 181 * {2.}_{}^{[ \frac{40 ° C . - 23 ° C .}{10} ]} = 588 days = 19 months$$

Q10 was the Reaction Rate Factor, which was set to the industry standard of 2.0; The temperatures used in the calculation were the target temperatures used during the study. The Accelerated Aging Temperature (AAT) was set to 40° C.; and TRT was the Real time Ambient Shelf Temperature which was set to 23° C. (based on the midpoint of USP <659>).

The AAT was calculated to be 588 days (19 months), which is representative of the ASTM F1980 under accelerated conditions and can be used to support 18 months of shelf life at the long-term conditions.

The data support the establishment of an 18-month shelf life for the fetal tissue product when stored under long-term storage conditions of −20° C.±5° C. to 25±2° C., 60±5% RH.

The long-term study continues as planned for the next 12 and 24 months with the addition of visual inspection and image capture of the product to ensure that the saline storage solution remains in the pouch and continues to maintain the hydration of the AM tissue.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

What is claimed:

1. A fetal tissue product comprising fetal tissue, wherein the fetal tissue product comprises less than about 30 micrograms of hyaluronic acid (HA) per 32 cm².

2. The fetal tissue product of claim 1, wherein the fetal tissue or the fetal tissue product is a sheet.

3. The fetal tissue product of claim 1, wherein the HA is low molecular weight HA, optionally wherein the low molecular weight HA comprises an average size of 500 kD or less.

4. The fetal tissue product of claim 1, wherein about 20% to about 50% of the HA is covalently bonded to a heavy chain 1 of IαI (HC1) complexed with pentraxin 3 (PTX3).

5. The fetal tissue product of claim 1, wherein about 20% to about 40% of the HA is covalently bonded to a heavy chain 1 of IαI (HC1) complexed with pentraxin 3 (PTX3).

6. The fetal tissue product of claim 1, comprising a total HA content of about 0.2 μg or less of HA.

7. The fetal tissue product of claim 1, wherein the fetal tissue comprises amniotic membrane (AM), optionally wherein the amniotic membrane is placental amniotic membrane.

8. The fetal tissue product of claim 2, wherein the sheet has a diameter from 8 millimeters to 16 millimeters.

9. The fetal tissue product of claim 2, wherein the sheet has an average thickness of less than about 200 micrometers.

10. The fetal tissue product of claim 2, wherein the sheet has an average thickness of greater than about 5 micrometers.

11. The fetal tissue product of claim 1, wherein the fetal tissue is not dehydrated.

12. The fetal tissue product of claim 1, wherein substantially all of the cells of the fetal tissue are dead.

13. The fetal tissue product of claim 1, wherein the fetal tissue product is terminally sterilized at a temperature at or below 0° C.

14. The fetal tissue product of claim 1, wherein the fetal tissue product is packaged in normal (0.9%) saline.

15. The fetal tissue product of claim 1, wherein the fetal tissue product is stable at 20° C. to 25° C. for at least one year.

16. The fetal tissue product of claim 1, wherein the fetal tissue product adheres to a substrate comprised of lotrafilcon A with a force of greater than 0.0001 pounds per square inch.

17. The fetal tissue product of claim 1, wherein the fetal tissue product is adhered to a contact lens or collagen shield such that when inserted into an eye of an individual the fetal tissue product contacts the surface of the eye of the individual.

18. An ophthalmic device comprising the fetal tissue product of claim 1, adhered or contacted to a substrate.

19. The ophthalmic device of claim 18, wherein the substrate comprises a collagen shield or a collagen corneal shield.

20. A method of treating a disease or disorder of an eye of an individual in need thereof comprising administering the fetal tissue product of claim 1, or the ophthalmic device of claim 18, to the eye of the individual.

21. The method of claim 20, wherein the disease or disorder of an eye is a disease or disorder of the surface of the eye.

22. The method of claim 20, wherein the disease or disorder of the eye comprises dry eye disease, corneal abrasion, superficial punctate keratitis, non-healing injury/wound, post debridement, epithelial basement membrane dystrophy, neurotrophic keratitis, corneal epithelial defect, corneal ulcer, or recurrent corneal epithelial erosion.

23. The method of claim 20, wherein the disease or disorder of the eye comprises Stevens-Johnson syndrome, glaucoma, macular hole, retinal detachment, macular degeneration or oculoplastic diseases.

24. The method of claim 20, wherein the disease or disorder of the eye comprises a burn, optionally wherein the burn is a chemical burn or a thermal burn.

25. The method of claim 20, wherein the disease or disorder of the eye comprises recovery from an eye surgery.

26. A method of making a fetal tissue product comprising obtaining a fetal tissue, subjecting it to terminal sterilization, isolating and recovering the fetal tissue to isolate the fetal tissue product of claim 1.

27. A kit for treating a disease or disorder of an eye, the kit comprising the fetal tissue product of claim 1 and one or more substrates, and, optionally, any one or more of saline or buffer, tweezers, swabs, forceps, or surgical trays.
